SEQUENCE LISTING (37 sequences)

SEQ ID NO: 1 - DNA, 87 bp, Homo sapiens
atgctttgcatacttctgcctgctggggagcctggggactttccacaccctaactgacacacattccacagctggttggtacctgca

SEQ ID NO: 2 - DNA, 8021 bp, Homo sapiens
  CDS (2379)..(4190)
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatatcaagcttatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagagcttatcgataccgtcgacacgtgtgatcagatgatccactagtaacggccgccagtgtgctggaattcgcggccgcggtgagaaccgtttccaccatggtcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttatggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacagtctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcaaacccttctcacctcggccgataagctctagaccaggccctggatccatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcattaagcttcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 3 - protein, 604 aa, Homo sapiens
MVARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATVWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 4 - DNA, 7342 bp, Homo sapiens
  CDS (2331)..(3530)
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatatcaagcttatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagagcttatcgataccgtcgacctcgagggatcagcttgcggccgcccaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagatctgggcccatcggatccatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcattaagcttcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 5 - protein, 400 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 6 - DNA, 7419 bp, Homo sapiens
  CDS (2315)..(3628)
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatatcaagcttatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagagcttatcgataccgtcgacgcggccgcccaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggccggggccatgccttgacccgacgccggagggccaagcgtgcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtctgctaaccgttacctggctatgaaggaagatggaagattactggcttctaaaaatgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaatggccgtcgagatccatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcattaagcttcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 7 - protein, 438 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGRGHALTRRRRAKRAAGSITTLPALPEDGGSGAFPPGHFKDPKRLYCKNGGFFLRIHPDGRVDGVREKSDPHIKLQLQAEERGVVSIKGVSANRYLAMKEDGRLLASKNVTDECFFFERLESNNYNTYRSRKYTSWYVALKRTGQYKLGSKTGPGQKAILFLPMSAKS

SEQ ID NO: 8 - DNA, 7522 bp, Homo sapiens
  CDS (2308)..(3678)
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattctggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatatcaagcttatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagagcttatcgataccgtcgacgccgccatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccagccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttcggatccatcgatagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcattaagcttcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgatcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgataccgtcgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 9 - protein, 457 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 10 - DNA, 1492 bp, Homo sapiens
gtcgacgccgccatggaatacccttatgatgtgccagattatgccgaattcccggggatcggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccagccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgaaagggcgaattctgcagatatcgcggccgctctagaccaggcgcctggatcc

SEQ ID NO: 11 - DNA, 589 bp, Homo sapiens
gtcgacgccgccatggaatacccttatgatgtgccagattatgccgaattcccggggatcggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatggacagccgctccgaccgctggtcccagatgccagcaagcagcggctgatggagaacacagaggactggcggtgaaagggcgaattctgcagatatcgcggccgctctagaccaggcgcctggatcc

SEQ ID NO: 12 - DNA, 814 bp, Homo sapiens
gtcgacgccgccatggaatacccttatgatgtgccagattatgccgaattcccggggatcggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgcctgaaagggcgaattctgcagatatcgcggccgctctagaccaggcgcctggatcc

SEQ ID NO: 13 - DNA, 9725 bp, Homo sapiens
ggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtaatcaagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccgacatcatgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactagaattggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacatgctttgcatacttctgcctgctggggagcctggggactttccacaccctaactgacacacattccacagctggttggtacctgcagtcgacatgctttgcatacttctgcctgctggggagcctggggactttccacaccctaactgacacacattccacagctggttggtacctgcagtcgacatgctttgcatacttctgcctgctggggagcctggggactttccacaccctaactgacacacattccacagctggttggtacctgcagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaaaggccgcgttgctggcgttggggataacgcaggaaagaacatgtgagcaaaaggccagcaaaaggccaggaaccgtaaatttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctcggtaccctacagttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcatgaattcgatatcgccgccatgatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagc

SEQ ID NO: 14 - DNA, 7745 bp, Homo sapiens
ggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacccgggctcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtagggcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccgacatcatgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaaatggacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatactagaattggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacatgctttgcatacttctgcctgctggggagcctggggactttccacaccctaactgacacacattccacagctggttggtacctgcagtcgacatgctttgcatacttctgcctgctggggagcctggggactttccacaccctaactgacacacattccacagctggttggtacctgcagtcgacatgctttgcatacttctgcctgctggggagcctggggactttccacaccctaactgacacacattccacagctggttggtacctgcagtcgacctcgagggggggcccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaaaggccgcgttgctggcgttggggataacgcaggaaagaacatgtgagcaaaaggccagcaaaaggccaggaaccgtaaatttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctcggtaccctacagttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcatgaattcgatatcgccgccatgatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagc

SEQ ID NO: 15 - DNA, 10 bp, Homo sapiens
  misc_feature (1)..(1): n is a, c, g, t or u
nccnccangg

SEQ ID NO: 16 - protein, 604 aa, Homo sapiens
MVARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVGKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 17 - RNA, 1405 nt, Homo sapiens
aagucuaaugaucauauuuauuuauuuauaugaaccaugucuauuaauuuaauuauuuaauaauauuuauauuaaacuccuuauguuacuuaacaucuucuguaacagaagucaguacuccuguugcggagaaaggagucauacuugugaagacuuuuaugucacuacucuaaagauuuugcuguugcuguuaaguuuggaaaacaguuuuuauucuguuuuauaaaccagagagaaaugaguuuugacgucuuuuuacuugaauuucaacuuauauuauaaggacgaaaguaaagauguuugaauacuuaaacacuaucacaagaugccaaaaugcugaaaguuuuuacacugucgauguuuccaaugcaucuuccaugaugcauuagaaguaacuaauguuugaaauuuuaaaguacuuuuggguauuuuucugucaucaaacaaaacagguaucagugcauuauuaaaugaauauuuaaauuagacauuaccaguaauuucaugucuacuuuuuaaaaucagcaaugaaacaauaauuugaaauuucuaaauucauaggguagaaucaccuguaaaagcuuguuugauuucuuaaaguuauuaaacuuguacauauaccaaaaagaagcugucuuggauuuaaaucuguaaaaucagaugaaauuuuacuacaauugcuuguuaaaauauuuuauaagugauguuccuuuuucaccaagaguauaaaccuuuuuagugugacuguuaaaacuuccuuuuaaaucaaaaugccaaauuuauuaaggugguggagccacugcaguguuaucucaaaauaagaauauccuguugagauauuccagaaucuguuuauauggcugguaacauguaaaaaccccauaaccccgccaaaagggguccuacccuugaacauaaagcaauaaccaaaggagaaaagcccaaauuauugguuccaaauuuagggugguuaaugaaguaccaagcugugcuugaauaacgauauguuuucucagauuuucuguuguacaguuuaauuuagcaguccauaucacauugcaaaaguagcaaugaccucauaaaauaccucuucaaaaugcuuaaauucauuucacacauuaauuuuaucucagucuugaagccaauucaguaggugcauuggaaucaagccuggcuaccugcaugcuguuccuuuucuuuucuucuuuuagccauuuugcuaagagacacagucuucucaaacacuucguuucuccuauuuuguuuuacuaguuuuaagaucagaguucacuuucuuuggacucugccuauauuuucuuaccugaacuuuugcaaguuuucagguaaaccucagcucaggacugcuauuuagcuccucuuaagaagauuaaaaaaaaaaaaaaag

SEQ ID NO: 18 - RNA, 97 nt, Homo sapiens
guccaggaacuccucagcagcgccuccuucagcuccacagccagacgcccucagacagcaaagccuacccccgcgccgcgcccugcccgccgcugcg

SEQ ID NO: 19 - DNA, 468 bp, Homo sapiens
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 20 - protein, 155 aa, Homo sapiens [sequence omitted]

SEQ ID NO: 21 - DNA, 1314 bp, Homo sapiens
atggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggccggggccatgccttgacccgacgccggagggccaagcgtgcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtctgctaaccgttacctggctatgaaggaagatggaagattactggcttctaaaaatgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagc

SEQ ID NO: 22 - protein, 438 aa, Homo sapiens; identical to SEQ ID NO: 7

SEQ ID NO: 23 - DNA, 852 bp, Homo sapiens
atggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggccggggccatgccttgacccgacgccggagggccaagcgt

SEQ ID NO: 24 - protein, 284 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGRGHALTRRRRAKR

SEQ ID NO: 25 - protein, 9 aa, Artificial sequence
  Amino acid sequence of an HA tag
YPYDVPDYA

SEQ ID NO: 26 - DNA, 33 bp, Artificial sequence
  Synthetic oligonucleotide PCR primer
gtcgacgccgccatggaatacccttatgatgtg

SEQ ID NO: 27 - DNA, 20 bp, Artificial sequence
  Synthetic oligonucleotide PCR primer
ggctcacacatgagagaagg

SEQ ID NO: 28 - DNA, 32 bp, Artificial sequence
  Synthetic oligonucleotide PCR primer
gtcgacgccgccatggattccttcaagtagtg

SEQ ID NO: 29 - DNA, 20 bp, Artificial sequence
  Synthetic oligonucleotide PCR primer
gaaatgcagggccacgactc

SEQ ID NO: 30 - DNA, 20 bp, Artificial sequence
  Synthetic oligonucleotide PCR primer
gcatacaggtcctggcatct

SEQ ID NO: 31 - DNA, 20 bp, Artificial sequence
  Synthetic oligonucleotide PCR primer
gctctcctgagctacagaag

SEQ ID NO: 32 - DNA, 21 bp, Artificial sequence
  Synthetic oligonucleotide PCR primer
ggttgctggtggtaggaatgt

SEQ ID NO: 33 - DNA, 21 bp, Artificial sequence
  Synthetic oligonucleotide PCR primer
ccagtaggcaggagaacatat

SEQ ID NO: 34 - DNA, 20 bp, Artificial sequence
  Synthetic oligonucleotide PCR primer
aaggcctccattgaccagag

SEQ ID NO: 35 - DNA, 20 bp, Artificial sequence
  Synthetic oligonucleotide PCR primer
cacttgcgttgatggtggct

SEQ ID NO: 36 - DNA, 1709 bp, Homo sapiens
agaacactggcggccgatcccaacgaggctccctggagcccgacgcagagcagcgccctggccgggccaagcaggagccggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccggtgctggccacaccagccccctttcacctccagtgccacaataaacctgtacccagctgtg

SEQ ID NO: 37 - protein, 457 aa, Homo sapiens; identical to SEQ ID NO: 9

(19) United States
(12) Patent Application Publication
(10) Pub. No.: US 2015/0240219 A1
(43) Pub. Date: Aug. 27, 2015

(54) METHODS FOR ACCELERATING BONE REPAIR

(71) Applicants: The United States Government, as represented by the Department of Veterans Affairs, Baltimore, MD (US); Loma Linda University, Loma Linda, CA (US)

(72) Inventors: David Baylink, Redlands, CA (US); Kin-Hing William Lau, Redlands, CA (US); Charles Rundle, Redlands, CA (US); Donna Strong, Beaumont, CA (US); Shin-Tai Chen, Colton, CA (US); Thomas Linkhart, Redlands, CA (US)

(73) Assignees: Loma Linda University; The United States Government, as represented by the Department of Veterans Affairs

(21) Appl. No.: 14/687,787

(22) Filed: Apr. 15, 2015

Related U.S. Application Data
(62) Division of application No. 11/503,365, filed on Aug. 10, 2006, now abandoned.
(60) Provisional application No. 60/707,732, filed on Aug. 11, 2005.

(51) Int. Cl. C12N 9/02

(52) U.S. Cl. CPC C12N 9/0083; C12Y 114/99001

(57) ABSTRACT

Vectors, such as retroviral vectors and transposon-based nonviral vectors, are disclosed herein that can be used to target transgene expression to the proliferating periosteal cells and cells in the marrow space after bone fracture. In one embodiment, these vectors include a human Cox-2 gene that is modified to improve mRNA stability and protein translation by truncating the 3′ untranslated region (UTR). In addition, in some embodiments, the native translation signal is replaced with an optimized Kozak sequence. These vectors can be used alone or with vectors expressing BMP2/4, FGF-2, or LMP-1 gene to repair bone fractures and increase prostaglandin secretion. Methods for identifying agents that accelerate bone repair are also disclosed.

### CROSS REFERENCE TO RELATED APPLICATIONS

This is a divisional of U.S. application Ser. No. 11/503,365, filed Aug. 10, 2006, which claims the benefit of U.S. Provisional Application No. 60/707,732, filed on Aug. 11, 2005. The prior applications are incorporated by reference herein in their entirety.

### FIELD

This application relates to the field of bone growth, specifically to the use of cyclooxygenase-2 (Cox-2) to accelerate fracture healing.

### BACKGROUND

Bone is a dynamic biological tissue composed of metabolically active cells that are integrated into a rigid framework. It is under a continuously occurring state of bone deposition, resorption and remodeling, processes that enable and facilitate bone regeneration and repair. The repair of bone is a complex, multi-step process involving proliferation, migration, differentiation, and activation of a number of cell types. Bone formation during the healing of fractures can occur through two distinct physiological processes. If bone segments are stabilized, or during development of some skull and facial bones and parts of the mandible and calvaria, mesenchymal precursor cells differentiate directly into bone-forming osteoblasts in a process called intramembranous ossification. Alternatively, in a biomechanically unstable environment, or in development of long bones of the appendicular skeleton and vertebrae of the axial skeleton, bone formation can occur through a cartilage intermediate in a process called endochondral ossification (see Mandracchia et al. Clin. Pod. Med. Surg. 18:55-77, 2001).

Bone metabolism is under constant regulation by a host of hormonal and local factors. The best known of these factors are the bone morphogenetic proteins (BMPs). BMPs are members of the transforming growth factor (TGF)-β superfamily (see Wozney and Rosen Clin. Orthop. Rel. Res. 346:26-37, 1998). Several members of this gene family have been identified. The major function of BMPs is to induce new bone formation. Numerous studies have demonstrated that BMPs can efficiently heal large bony defects as well as segmental defects. Recombinant proteins have now been generated and are undergoing clinical trials. BMP-2, when combined with inactivated de-mineralized bone matrix used as a carrier, has been shown to induce de novo cartilage and bone formation in rat, sheep and dog bone defect models (see Lee et al. J. Biomed. Mater. Res. 28:1149-1156, 1994, amongst others). BMP-7 has also been shown to heal large segmental defects in animal models (see Cook et al., J. Bone Joint Surg. 76A:827-838, 1994).

It is estimated that 5.6 million fractures occur annually in the United States alone, and, despite advances in surgical techniques, about 5-10% of these result in delayed or impaired healing, known as delayed unions or non-unions. Furthermore, the majority of non-unions (up to 80%) are atrophic (avascular, see Einhorn, J. Bone Joint Surg. 77:940-956, 1995). Failure of proper and complete fracture healing results in pain, instability, and associated loss of functions of the suffering limb. Because a significant number of fractures occur in productive individuals young and old, the degree of disability caused by fractures and non-unions is substantial. Thus, enhancement of the fracture repair process would be of great benefit to ensure the rapid restoration of skeletal function. The ability of injured patients to return to the work force or to recreational activities has an economic impact on society and would also improve the overall physical and mental well-being of the patients.

### SUMMARY

Disclosed herein are vectors that can be used to target expression of nucleic acids encoding cyclooxygenase-2 (Cox-2). These vectors can be used, for example, to target Cox-2 transgene expression to the proliferating periosteal cells arising shortly after bone fracture. The vectors include a human Cox-2 gene that is modified to improve mRNA stability and protein translation by truncating the 3′ untranslated region (UTR). In addition, in some embodiments, the native Kozak translation signal is replaced with an optimized Kozak sequence. Viral and non-viral vectors encoding Cox-2 are disclosed. These vectors encoding Cox-2 can be used to increase prostaglandin secretion and repair bone fractures. In several embodiments the Cox-2 expressing vector can be alone, or in combination with nucleic acids encoding a bone morphogenic proteins (such as BMP-4), a fibroblast growth factor (such as FGF-2), or LIM mineralization Proteins (such as LIM-1) to accelerate bone fracture repair or other bony defects.

In additional embodiments, methods for identifying agents of use in repairing bone fractures and/or accelerating spinal fusion are also disclosed herein.

The foregoing and other features and advantages will become more apparent from the following detailed description of several embodiments, which proceeds with reference to the accompanying figures.

### BRIEF DESCRIPTION OF THE FIGURES

FIG. 1 is a schematic diagram of the structure of the MFG-like pCLSA-human (h)Cox-2 murine leukemia virus (MLV) retroviral expression vector (also is referred to as the pCLSA-hCox-2 vector elsewhere in this disclosure). The full length hCox-2 cDNA coding region with an optimized Kozak sequence was ligated into the 6.8-kb retroviral vector backbone. The viral splice donor (SD) and splice acceptor (SA) are retained as well as a packaging signal (ψ) within the viral Gag gene. Unique regions (U3, U5) and the repetitive region (R) are retained to provide the transcription start site and the polyadenylation signal. Three Simian Virus (SV)40 origins of replication (filled boxes) are included to increase the viral titer in SV40 large T antigen expressing 293 T cells.

FIGS. 2A-2D are digital images of Western immunoblot analysis of Cox-2 expression,and bar graphs showing PGE2 production and alkaline phosphatase (ALP) activity in rat marrow stromal cells and rat calvarial osteoblasts after transduction with MLV-hCox-2. Rat marrow stromal cells (rMSCs), rat calvarial osteoblasts (rCOBs) were transduced with MLV-hCox-2 or MLV-β-gal control. For the results shown in FIG. 2A: At one week post-transduction with MLV-hCox-2, the expression of the intact 72-kDa human (h) Cox-2 protein in the MLV-hCox-2-treated, the MLV-β-gal-treated, or untransduced cells were measured with the Western immunoblot assay. The levels of the 42-kDa actin in each cell type were also measured as a reference of protein loading. For the results shown in FIG. 2B, the effects of cell passage on the expression of hCOX-2 in the MLV-hCox-2-transduced rMSCs and rCOBs were determined by measuring the relative Cox-2 expression level in cells after passage 1 (P1, 7 days in culture), passage 3 (P3, 21 days in culture), or passage 5 (P5, 35 days in culture) with Western immunoblots. For the results shown in FIG. 2C, conditioned medium (CM) levels of a product of Cox-2, PGE₂ (a prostaglandin that stimulates bone formation) were measured in rMSC and rCOBs after transduction. Values are the mean±S.E.M. (n=4). For the results shown in FIG. 2D, rMSCs and rCOBs that were transduced with MLV-Cox 2 or MLV-β-gal were assayed for cellular ALP activity (normalized against cellular protein) at confluence. Values are the mean±S.E.M. (n=6). Data are representative of 3-5 independent experiments.

FIGS. 3A-3H are a set of digital images showing the healing of fractures injected with the Cox-2 transgene (FIGS. 3A, 3C, 3E, 3G) or the β-galactosidase control transgene (FIGS. 3B, 3D, 3F, 3H) at 7 days (FIGS. 3A, 3B), 10 days (FIGS. 3C, 3D) 17 days (FIGS. 3E, 3F), or 21 days (FIGS. 3G, 3H) post-fracture. Sections were stained with Van Giesen stain. Insets are X-rays of the fracture tissues at each time (Scale bar=1 cm). The empty marrow space is where the pin held the fracture in place before removal at harvest (Scale bar=200 μm). Union of bony osteoid tissues at the fracture gap was accelerated at 17 days in the MLV-Cox-2 animals as compared to control fractures. There was also a reduction in the amount of cartilage in the Cox-2-treated fracture. The MLV-beta-galactosidase-injected fracture is typical of normal fracture repair.

FIG. 4A-4B are graphs of the results obtained from real-time RT-PCR analysis of Cox-2 expression in the fracture. The human Cox-2 transgene and β-galactosidase control gene were transfected into fracture tissues using MLV-based vector. Endogenous rat Cox-2 gene (FIG. 4A) and human Cox-2 transgene (FIG. 4B) expression were determined by real-time RT-PCR with species-specific primers at several time points (4, 7, 14 and 21 days after fracture following the injection of the corresponding vector). Endogenous rat Cox-2 and human Cox-2 transgene expression were compared with expression of the housekeeping gene cyclophilin in each corresponding sample. Values represent the mean±S.E.M. of duplicate measurements of each cDNA preparation of at least four individual rats for each vector at each post-fracture time point. In FIG. 4A, the endogenous rat Cox-2 gene expression levels in the fracture tissues of rats injected with the MLV-hCox-2 vector were not significantly different from those tissues transfected with the β-galactosidase control gene, indicating that endogenous Cox-2 gene expression was not altered by Cox-2 transgene expression. In FIG. 4B, the control fractures receiving the MLV-β-galactosidase control gene did not show any degree of human(h)Cox-2 gene expression. In the fracture that was injected with the MLV-hCox-2 vector, transgene expression significantly increased human Cox-2 mRNA expression at each post-fracture time (p<0.05). The increase was sustained throughout the 21-day observation period.

FIG. 5 is a digital image of a Western immunoblot showing that abundant full length HA-tagged LMP-1 is produced by HT1080 cells transduced with the MLV-HA-LMP-1 virus. The Western immunoblot was treated with mouse monoclonal anti-HA-antibody and HA-tagged LMP-1 was identified with HPR labeled goat anti-mouse IgG in the cell extract as a 53 kDa protein (arrow) using chemiluminescent reagents. The HA-tagged band was not found in MLV-GFP transduced cells or in LMP-1 transduced cells.

FIG. 6 are digital images that compare mineralized tissues in the fracture callus in response to injection of MLV-LMP1 and the osteogenic gene MLV-BMP2/4 at 28 days. Fractures were injected through the intramedullary cavity via catheter with MLV-LMP1 therapeutic gene (top), MLV-BMP2/4 therapeutic gene (middle) or the MLV-β-galactosidase control gene (bottom). The femurs compared by X-ray analysis at 28 days healing. Hard callus mineralization in the MLV-LMP1-injected fracture tissues was comparable to that of the MLV-BMP2/4-injected fracture tissues and greater than control tissues, suggesting that LMP1 actions were similar to osteogenic differentiation promoted by BMP2/4. MLV-HA-tagged LMP-1 and LMP-1 viral vectors increased osteogenic differentiation at 21 days. Scale bar=1 cm.

FIGS. 7A-7B are digital images that show the development of mineralized tissues in the fracture callus in response to intramedullary injection of MLV-based vectors expressing FGF-2 genes. To increase FGF-2 secretion from transfected cells, the FGF-2 gene was modified by the addition of a bone morphogenetic protein (BMP)-2 signal sequence and by the substitution of cysteines 70 and 88 by serine and asparagine, respectively. FIG. 7A shows the gross anatomy of the healing fracture callus development at 11 days of healing after intramedullary injection of MLV-FGF-2 (double mutant, top panel of FIG. 7A), MLV-FGF-2 (wild-type, middle panel of FIG. 13A) or the control MLV-green fluorescent protein marker gene (MLV-GFP, bottom panel of FIG. 7A). The double mutant FGF-2 gene produced a massive fracture callus, while the FGF-2 wild-type gene produced a much smaller fracture callus more typical of the normal healing observed with the non-therapeutic control gene. FGF-2 gene expression from transfected cells dramatically altered fracture callus maturation; healing of greater therapeutic value can be obtained by optimizing the viral vector dosage for FGF-2 gene expression. Scale bar=1 cm. FIG. 7B shows the X-ray analysis of the healing calluses at 11 days healing. The double mutant FGF-2 gene (top panel of FIG. 7B) produced a massive fracture callus with evidence of increased mineralized tissues, which were not visible in the hard callus of the wild-type FGF-2 gene (middle panel of FIG. 7B) or in that of the non-therapeutic control gene fractures (bottom panel of FIG. 7B). FGF-2 gene expression from transfected cells produced mineralized tissue, although most of the callus was nonmineralized. Scale bar=1 cm. Thus, mineralized tissue development and bone formation can be increased by optimizing the viral vector dosage for FGF-2 gene expression.

FIG. 8 are digital images showing the comparison of the localization of cells within the fracture site transduced with MLV-based vectors with those transduced with lentiviral (HIV-based) vectors. The intramedullary space of the fracture was injected at one day post-fracture with 0.1 ml of an approximately 1×10⁷ transforming units MLV-based (left panel) or lentiviral-based (right panel) vectors expressing the β-galactosidase marker gene through a catheter. The femurs were harvested at one week post-fracture and stained for β-galatosidase expression. Each femur was split open to enhance stain penetration. Both outside (left panels) and inside (right panels) aspects are shown. Scale bar=1 cm.

FIG. 9 is a digital image of an X-ray showing that at 21 days significant bone formation has occurred in a fractured rat femur injected with either MLV-BMP2/4 virus or lentiviral-BMP2/4 virus via catheter. A comparison of was made of an MLV-based vector expressing the BMP-2/4 therapeutic transgene with lentiviral-based vector expressing BMP-2/4 transgene to enhance bone formation during fracture healing. The intramedullary space of the fracture was injected at one day post-fracture with 0.1 ml of 1×10⁷ transforming units (tfu) MLV-based BMP-2/4 gene (left panel), MLV-based β-galactosidase control gene (center panel) or lentiviral-based BMP-2/4 gene (right panel) vectors expressing the BMP-2/4 gene. Bridging of the fracture gap was obvious in the whole animal X-ray for either vector and BMP-2/4 gene combination (top left and top right), but a fracture gap was still obvious in the MLV-β-galactosidase control gene injection (top center). The femurs harvested at three weeks post-fracture and examined at higher resolution for mineralized tissues by X-ray (bottom). The unfractured contralateral femur from the same animal is presented for comparison. The isolated bones were obtained from different animals from those in the live animal X-rays above. Scale bar=1 cm.

FIGS. 10A-10B are schematic diagrams of the Tc1-like transposon-based Prince Charming (pPC) nonviral vectors containing the SV40 DNA nuclear targeting sequence (SV40dts) with (FIG. 10A) or without (FIG. 10B) the Neo selection gene. Prince Charming nonviral vector (FIG. 10A) is a single plasmid-based version of the Sleeping Beauty Tc1-like transposon-based nonviral vector (see Harris et al. Anal. Biochem. 310:15-26, 2002). To construct this vector expressing the BMP2/4 gene, the SV40dts (5′-atgctttgcatacttctgcctgctggggagcctggggactttccacaccctaactgacac acattccacagctggttgg acctgca-3′, SEQ ID NO: 1) was inserted 1, 2 or 3 times in tandem as Sall fragments generated by PCR in a forward orientation in the SalI site of the pPC-BMP2/4 vector. This site is outside the BMP2/4 expression cassette and outside the transposon IR/DR(R) repeats in the pPC nonviral vector. The BMP2/4 coding sequence was inserted in the EcoRV/NotI site to replace the NLS-Red Fluorescent Protein coding sequence in the pPC-RFP construct (see Harris et al., Anal. Biochem. 310:15-26, 2002). This vector is called pPC-Neo-BMP-2/4-SV40DTS(1-3) (9.7 kb). A second 7.7 kb construct (FIG. 10B) called pPC-BMP2/4-SV40DTS(1-3) was prepared with the Neo cassette removed (5′ of the F1 origin to 5′ of the IR/DR(R) repeat. A unique SmaI site was included.

FIGS. 11A-11B are bar graphs showing that the pPC-BMP-2/4 nonviral vector significantly increases ALP activity in ROS 17/2.8 osteoblastic cells (FIG. 11A) and C2C12 myogenic precursor cells (FIG. 11B). In the right panel, transfection of ROS 17/2.8 cells was performed with Effectene and the pPC-BMP2/4 vectors. While the pPC-BMP2/4 vector increases BMP2/4 expression to increase ALP activity, this vector with one, two, or three copies of the SV40 dts increases expression of ALP activity in differentiated osteoblasts up to 14-fold compared to the pPC-NLS-RFP vector control. Two copies of the SV40dts were much less efficient. C2C12 cells are myogenic precursor cells that can be reprogrammed to become osteoblasts with BMP2/4. Cells were transfected and cultured for 5 days in order to allow development of the osteoblast phenotype. The pPC-BMP2/4 vector did not significantly change ALP activity in C2C12 cells but this vector containing 1, 2, or 3 copies of the SV40dts inserted into the unique Sall site significantly increased ALP activity (1.6 and 2 fold) and the osteoblast phenotype. Similarly, two copies of SV40 dts was less effective and increased ALP activity by only 20% of control.

FIGS. 12A-12D are the complete nucleic sequences of one of the MLV-based pCLSA-vectors used in many of the various examples of this disclosure, pCLSA-hCox2. The nucleic acid sequence (SEQ ID NO: 2) is 8021 base pairs, the amino acid sequence (SEQ ID NO: 3) is shown below the nucleic acid sequence. The italic nucleotide sequence is the open reading frame corresponding to the protein coding region. The bold sequences are the Sall restriction site and the BamHI restriction site. The optimized Kozak sequence is underlined. The amino acid sequence of the open reading frame of the human Cox2 is shown underneath the nucleic acid sequence. Additional forms of LMP-1 are presented only as the SalI-BamHI fragments that were inserted into the MLV-based retroviral vector. The italic sequences are the protein coding regions.

FIGS. 13A-13D are the complete nucleic sequences of one of the MLV-based pCLSA-vectors used in many of the various examples of this disclosure, pCLSA-hBMP2/4 (7342 base pairs). The nucleic acid sequence (SEQ ID NO: 4) is 7342 base pairs, the amino acid sequence (SEQ ID NO: 5) is shown below the nucleic acid sequence. The italic nucleotide sequence is the open reading frame corresponding to the protein coding region. The bold sequences are the SalI restriction site and the BamHI restriction site. The optimized Kozak sequence is underlined. The underlined amino acid sequences are that of the signal sequence of BMP2 and that of the remnant sequence of the BMP4 signal sequence. The bold amino acid sequence is that of the mature BMP4 protein.

FIGS. 14A-14D are the complete nucleic acid sequences (SEQ ID NO: 6) of one of the MLV-based pCLSA-vectors used in many of the various examples of this disclosure, pCLSA-BMPFGFC2SC3N (7419 bp). The italicized nucleic acid sequence (of SEQ ID NO: 6) is the inserted BMP2/4-FGF-2 gene. The rest of the nucleic sequence (of SEQ ID NO: 6) is that of the MLV vector sequence. The amino acid sequence is also shown (SEQ ID NO: 7). The underlined amino acid sequence is the BMP2/4 signal sequence. The bold letters are derived from the signal sequence of BMP2, and the bold and underlined letters are the remnant sequence of the BMP4 signal sequence. The bold amino acid sequence is that of the C2SC3N modified human FGF-2 gene. The boxes denote the location of the cys-70 to ser mutation and cys-88 to asparagine mutation, respectively.

FIG. 15A-15D are the nucleic acid sequence (SEQ ID NO: 8) of one of the MLV-based pCLSA vectors used in the various examples of this disclosure, pCSLA-hLMP1, which is 7522 base pairs. The amino acid sequence (SEQ ID NO: 9) is also shown.

FIG. 16 is the nucleic acid sequence (SEQ ID NO: 10) of HA-tagged hLMP-1 (Sal I/Bam HI insert-Met¹-Val⁴⁵⁷).

FIG. 17 is the nucleic acid sequence (SEQ ID NO: 11) of HA-tagged hLMP-1 (SalI/Bam HI insert-Met¹-Arg¹⁵⁶).

FIG. 18 is the nucleic acid sequence (SEQ ID NO: 12) of HA-Tagged hLMP1 (SalI/Bam HI insert-Met¹-A²³¹).

FIGS. 19A-19D are the nucleic acid sequence (SEQ ID NO: 13) of pPC-Neo-BMP-2/4-SV40DTS3 (9725 bp). The Not I and EcoRV site are in bold and frame the BMP-2/4 coding sequence. The BMP-2/4 nucleotide sequence is in italics. The optimized Kozak sequence is underlined. The three copies of the SV40 DTS at the Sal I site are in bold. The Neomycin resistance expression cassette is intact.

FIGS. 20A-20C are the nucleic sequence (SEQ ID NO: 15) of pPC-BMP-2/4-SV40DTS3 (7745 bp). The Not I and EcoRV site are in bold and frame the BMP-2/4 coding sequence. The BMP-2/4 nucleotide sequence is in italics. The optimized Kozak sequence is underlined. The three copies of the SV40 DTS at the Sal I site are in bold. There is no Neomycin resistance expression cassette and a unique SmaI site (bold) was introduced in its place.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜128 kb), which was created on Apr. 14, 2015, and is incorporated by reference herein. In the accompanying Sequence Listing:

SEQ ID NO: 1 is the nucleic acid sequence of a DNA targeting sequence (DTS).

SEQ ID NO: 2 is the nucleic acid sequence of pCLSA-hCox2.

SEQ ID NO: 3 is the amino acid sequence encoded by pCLSA-hCox2.

SEQ ID NO: 4 is the nucleic acid sequence of pCLSA-hBMP2/4.

SEQ ID NO: 5 is the amino acid sequence encoded by pCLSA-hBMP2/4.

SEQ ID NO: 6 is the nucleic acid sequence of pCLSA-BMPFGFC2SC3N.

SEQ ID NO: 7 is the amino acid sequence encoded by pCLSA-BMPFGFC2SC3N.

SEQ ID NO: 8 is the nucleic acid sequence of pCSLA-hLMP1.

SEQ ID NO: 9 is the amino acid sequence encoded by pCSLA-hLMP1.

SEQ ID NO: 10 is the nucleic acid sequence of HA-tagged H-LMP-1 (Sal I/Bam HI insert-Met^{1‘-Val}⁴⁵⁷).

SEQ ID NO: 11 is the nucleic acid sequence of HA-tagged hLMP-1 (Sal I/Bam HI insert-Met¹-Arg¹⁵⁶).

SEQ ID NO: 12 is the nucleic acid sequence of HA-Tagged hLMP1 (SalI/Bam HI insert-Met¹-A²³¹).

SEQ ID NO: 13 is the nucleic acid sequence of pPC-Neo-BMP-2/4-SV40DTS3.

SEQ ID NO: 14 is the nucleic acid sequence of pPC-BMP-2/4-SV40DTS3.

SEQ ID NO: 15 is an optimized Kozak nucleic acid sequence.

SEQ ID NO: 16 is the amino acid sequence of a human Cox2.

SEQ ID NO: 17 is the nucleotide sequence of a region of the 5′ UTR of human Cox2.

SEQ ID NO: 18 is the nucleotide sequence of a region of the 3′ UTR of human Cox2.

SEQ ID NO: 19 is the nucleotide sequence encoding a human FGF-2.

SEQ ID NO: 20 is the amino acid sequence of a human FGF-2.

SEQ ID NO: 21 is the nucleotide sequence encoding a human FGF-2 analog.

SEQ ID NO: 22 is the amino acid sequence of a human FGF-2 analog.

SEQ ID NO: 23 is the nucleotide sequence encoding a BMP-2/4 secretion signal sequence.

SEQ ID NO: 24 is the amino acid sequence of a BMP-2/4 secretion signal sequence.

SEQ ID NO: 25 is the amino acid sequence of an HA tag.

SEQ ID NOs: 26-35 are the nucleotide sequences of primers.

SEQ ID NO: 36 is the nucleotide sequence encoding a human LMP-1.

SEQ ID NO: 37 is the amino acid sequence of a human LMP-1.

### DETAILED DESCRIPTION

### I. Abbreviations

ALP: alkaline phosphatase

BMP: bone morphogenic protein

CMV: cytomegalovirus

Cox2: cyclooxygenase 2

DNA: deoxyribonucleic acid

DTS: DNA nuclear targeting sequence

Env: envelope

Gag: glycosaminoglycan

GFP: green fluorescent protein

FBS: fetal bovine serum

LMP: LIM mineralization protein

MLV: Moloney leukemia virus

FGF: fibroblast growth factor

HA: hemaglutinin

IL: interleukin

LTR: long terminal repeat

PBS: phosphate buffered saline

PCR: polyermase chain reaction

Pol: polymerase

SV40: simian virus 40

TGF: transforming growth factor

UTR: untranslated region

### I. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). In order to facilitate review of the various embodiments of this disclosure, the following explanations of specific terms are provided:

Administration: The introduction of a composition into a subject by a chosen route. For example, if the chosen route is intravenous, the composition is administered by introducing the composition into a vein of the subject. If the chosen route is intraarticular, the composition is administered by introducing the composition into a joint of the subject.

Amplification: Of a nucleic acid molecule (e.g., a DNA or RNA molecule) refers to use of a technique that increases the number of copies of a nucleic acid molecule in a specimen. An example of amplification is the polymerase chain reaction, in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in PCT Publication No. WO 90/01069; ligase chain reaction amplification, as disclosed in EP Application No. EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Animal: Living multi-cellular vertebrate organisms, a category that includes, for example, mammals and birds. The term mammal includes both human and non-human mammals. Similarly, the term “subject” includes both human and veterinary subjects.

Bone disease: Includes any disease, defect, or disorder which affects bone strength, function, and/or integrity, such as decreasing bone tensile strength and modulus. Examples of bone diseases include, but are not limited to, diseases of bone fragility, such as osteoporosis and genetic diseases which result in abnormal bone formation such as McCune-Albright syndrome (MAS) and osteogenesis imperfecta. Other examples of bone diseases include malignancies and/or cancers of the bone such as a sarcoma, such as osteosarcoma.

Bone-forming cells and mineral forming cells: Cells having osteogenic potential. Examples include, but are not limited to: bone marrow stromal cells, osteoblasts, osteocytes, and dental pulp cells. “Osteogenesis” is the formation or production of bone.

Bone Healing and Fracture Healing: Bone heals (fuses) in a unique way compared with other connective tissues. Rather than develop scar tissue, it has the ability to regenerate itself completely. The majority of fractures heal by secondary fracture healing and that involves a combination of intramembranous and endochondral ossification. Without being bound by theory, it is generally believed that the fracture healing sequence involves five discrete stages of healing. This includes an initial stage in which a haematoma is formed and inflammation occurs; a subsequent stage in which cartilage begins to form and angiogenesis proceeds, and then three successive stages of cartilage calcification, cartilage resorption and bone deposition, and ultimately a more chronic stage of bone remodeling. Generally, committed osteoprogenitor cells and uncommitted, undifferentiated mesenchymal stem cells contribute to the process of fracture healing. Bone that forms by intramembranous ossification is found early and further from the site of the fracture, results in the formation of a hard callus, and forms bone directly without first forming cartilage. Generally, two weeks after fracture, cell proliferation declines and hypertrophic chondrocytes become the dominant cell type in the chondroid callus. The resulting endochondral bone is formed adjacent to the fracture site.

Bone Morphogenic Proteins (BMPs): A family of proteins, identified originally in extracts of demineralized bone that were capable of inducing bone formation at ectopic sites. BMPs are found in minute amounts in bone material (approximately 1 microgram/kg dry weight of bone). Most members of this family (with the exception of BMP-1) belong to the transforming growth factor-β family of proteins.

BMPs can be isolated from demineralized bones and osteosarcoma cells. They have been shown also to be expressed in a variety of epithelial and mesenchymal tissues in the embryo. BMPs are proteins which act to induce the differentiation of mesenchymal-type cells into chondrocytes and osteoblasts before initiating bone formation. They promote the differentiation of cartilage- and bone-forming cells near sites of fractures but also at ectopic locations. Some of the proteins induce the synthesis of alkaline phosphatase and collagen in osteoblasts. Some BMPs act directly on osteoblasts and promote their maturation while at the same time suppressing myogenous differentiation. Other BMPs promote the conversion of typical fibroblasts into chondrocytes and are capable also of inducing the expression of an osteoblast phenotype in non-osteogenic cell types. BMPs include BMP-1 to BMP-15, such as BMP-2 and BMP-4. BMP-2 and BMP-4 and BMP-7 have been shown to promote bone formation. BMP2/4 is a hybrid gene in which the secretion signal of BMP4 is replaced with that of BMP2 (see Peng et al., Mol. Therapy 4:95-104, 2001, incorporated herein by reference).

cDNA (complementary DNA): A piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

Conservative Substitutions: Modifications of a polypeptide that involve the substitution of one or more amino acids for amino acids having similar biochemical properties that do not result in change or loss of a biological or biochemical function of the polypeptide are designated “conservative” substitutions. These conservative substitutions are likely to have minimal impact on the activity of the resultant protein. Table 1 shows amino acids that can be substituted for an original amino acid in a protein, and which are regarded as conservative substitutions.

TABLE 1

| Original Residue | Conservative Substitutions |
|---|---|
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

One or more conservative changes, or up to ten conservative changes (such as two substituted amino acids, three substituted amino acids, four substituted amino acids, or five substituted amino acids, etc.) can be made in the polypeptide without changing a biochemical function of the osteogenic growth factor, such as Cox-2, LIM-1 or FGF-2.

Contacting: Placement in direct physical association. Includes both in solid and liquid form.

Cyclooxygenase (Cox): An enzyme protein complex present in most tissues that catalyses two steps in prostaglandin biosynthesis and produces prostaglandins and thromboxanes from arachidonic acid. Cox-2 is also known as prostaglandin-endoperoxide synthase (PTGS), and is a key enzyme in prostaglandin biosynthesis. The cyclooxygenase activity converts arachidonate and 2O₂ to prostaglandin G₂; the hydroperoxidase activity uses glutathione to convert prostaglandin G₂ to prostaglandin H₂. Cyclooxygenase activity is inhibited by aspirin like drugs, accounting for their anti-inflammatory effects. Cyclooxygenase (Cox) exists as two isozymes, Cox-1 and Cox-2. Cox-2, but not Cox-1, is an inducible enzyme and its expression is highly regulated. Both isozymes form prostaglandins that support physiologic functions; however, the formation of proinflammatory prostaglandins is catalyzed by Cox-2 Inhibition of Cox-2 accounts for the anti-inflammatory and analgesic action of non-steroidal anti-inflammatory drugs (NSAIDs).

Cytokine: The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor-α, interleukin (IL)-6, IL-10, IL-12, transforming growth factor, and interferon-γ.

Degenerate variant: A polynucleotide encoding a Cox-2 polypeptide that includes a sequence that is degenerate as a result of the genetic code. There are 20 natural amino acids, most of which are specified by more than one codon. Therefore, all degenerate nucleotide sequences are included as long as the amino acid sequence of the Cox-2 polypeptide encoded by the nucleotide sequence is unchanged.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

DNA Nuclear Targeting Sequence (DTS): A specific DNA sequence or repeats of a DNA sequence that is needed to support nuclear import of an otherwise cytoplasmically localized plasmid DNA. Naturally occurring DNA sequences in the promoters of viruses or in the promoters of mammalian genes provide nuclear entry of the DNA containing a transgene by incorporating them into plasmid-expression vectors that can be expressed in a non-dividing cell. A non-limiting example is the DNA sequence from the SV40 genome, which contain the 72 by enhancer repeats (5′-atgctttgca tacttctgcc tgctggggag cctggggact ttccacaccc taactgacac acattccaca gctggttggt acctgca-3′, SEQ ID NO: 1). This SV40 DTS has been shown to support sequence-specific DNA nuclear import of plasmid DNA (see Dean et al., Exp. Cell Res. 253:713-722, 1999).

Expressed: The translation of a nucleic acid sequence into a protein. Proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Fibroblast Growth Factor (FGF): A large family of multigene family of growth factors that is a pleiotropic regulator of the proliferation, differentiation, migration, and survival in a variety of cell types (see Bikfalvi et al., Endocrine Rev. 18:26-45, 1997). The proteins in this family are 16-18 kDa proteins controlling normal growth and differentiation of mesenchymal, epithelial, and neuroectodermal cell types.

Two main groups of FGF are known. One type of FGF was isolated initially from brain tissue and identified by its ability to enhance proliferation of murine fibroblasts. Due to its basic pI the factor was named basic FGF or FGF-2 (see below) This factor is the prototype of the FGF family. Another factor, isolated also initially from brain tissues, has the ability to enhance proliferation of myoblasts. This factor is termed acidic FGF (aFGF). Other proteins in the FGF family are int-2 (FGF-3), FGF-4 FGF-5, FGF-6, K-FGF (FGF-7) and FGF-8. All of these factors are products of different genes. Some FGF are not secreted (FGF-2) while others (FGF-3, FGF-4, FGF-5 and FGF-6) have a signal sequence and therefore are secreted. Presently there are 23 factors identified as an FGF (numbered FGF-1 to FGF-23).

Basic fibroblast growth factor (“b-FGF” or “FGF-2”) is a potent stimulator of angiogenesis (see D'Amore and Smith, Growth Factors 8:61-75, 1993) and hematopoiesis in vivo (see Allouche and Bikfalvi, Prog. Growth Factor Res. 6:35-48, 1995). FGF-2 is also involved in organogenesis (Martin, Genes Dev. 12:1571-1586, 1998), vascularization (see Friesel and Maciag, FASEB J. 9:919-925, 1995), and wound healing (see Ortega et al., Proc. Natl. Acad. Sci. USA 95:5672-5677, 1998), and plays an important role in the differentiation and/or function of various organs, including the nervous system (see Ortega et al., Proc. Natl. Acad. Sci. USA 95:5672-5677, 1998), and the skeleton (see Montero et al., J. Clin. Invest. 105:1085-1093, 2000). Because of its angiogenic and anabolic properties, FGF-2 has been shown to be involved in wound healing.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, neutrophil, macrophage or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Inhibiting or treating a disease: Inhibiting the full development of a disease or condition or accelerating healing, for example, in a subject who is at risk for a disease (for example, atherosclerosis or cancer). “Treatment” refers to a therapeutic intervention that ameliorates a sign or symptom of a disease or pathological condition after it has begun to develop. Treatment can also refer to acceleration of fracture healing. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, such as pain, a shortened recovery time or an improvement in the overall health or well-being of the subject, or by other parameters well known in the art that are specific to the particular disease.

Isolated: An “isolated” biological component (such as a nucleic acid or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Kozak Sequence: Sequences flanking the AUG initiation codon within mRNA that influence the recognition of the initiation codon by eukaryotic ribosomes. The optimized Kozak sequence used in many of the embodiments of this disclosure is X₁CCX₂CCAUGG (SEQ ID NO: 15, wherein X₁ and X₂ can be any base). As a result of studying the conditions required for optimal translational efficiency of expressed mammalian genes, the ‘Kozak’ consensus sequence has been identified. It has been proposed that this defined translational initiating sequence (⁻⁶GCC [symbol] G⁺⁴, SEQ ID NO: 15, wherein X₁ is a G and X₂ is an A or a G) can be included in vertebrate mRNAs located around the initiator codon to enhance translation. Efficient translation is obtained when an optimized Kozak sequence is utilized. Optimized Kozak sequences include those wherein the −3 position contains a purine base or, in the absence of a purine base, a guanine is positioned at +4. One example of an optimized Kozak sequence is TCCACCAUGG (SEQ ID NO: 15, wherein X₁ is a T and X₂ is an A). Another example of an optimized Kozak sequence is a nucleotide sequence comprising ACCAUGG, such as GCCACCAUGG (SEQ ID NO: 15, wherein X₁ is a G and X₂ is an A), where the A in the underlined AUG start codon is coordinate 1 and the A at position −3 could also be a G. A purine (usually A) in position −3 results in efficient initiation of translation, and in its absence, a G at position +4 results in efficient initiation.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes.

LIM Mineralization Protein (LMP): LMP-1 is a gene induced by glucocorticoids in rat calvaria osteoblasts (see Boden et al., Endocrinology 139:5125-5134, 1998), also called Enigma (see Wu and Gill, J. Biol. Chem. 269:25085-25090, 1994; Jurata and Gill, Curr. Top. Microbiol. Immunol. 228:75-113, 1998; Gill, Structure 3:1285-1289, 1995) that includes the LIM cytosine rich zinc binding domain. For main groups of LIM domain proteins are known (Jurata et al., Curr. Top. Microbiol. Immunol. 228: 75-113, 1998). One type of LIM-domain protein in the LIM homeodomain group, was found to mediate binding interactions in fibroblasts with many other important proteins involved in mitogenic signaling (Wu and Gill, J. Biol. Chem. 269:25085-25090, 1994). Overexpression of LMP-1 in rat calvarial cells by plasmid vector transfection stimulated osteoblast differentiation and stimulated secretion of growth factors that stimulated osteoblast differentiation (see Boden et al., Endocrinology 139:5125-5134, 1998; Liu et al., J. Bone Miner. Res. 17:406-414, 2002). LMP-1 has been shown to stimulate bone formation in vivo in a rabbit spinal fusion model (see Viggeswarapu et al., J. Bone Joint Surg. Am. 83-A:364-376, 2001; Yoon et al., Spine 29:2603-2611, 2004). LMP-1 increases expression of proteoglycan, BMP-2, BMP-4 and BMP-7 (see Yoon et al., Spine 29:2603-2611, 2004; Minamide et al., J. Bone Joint Surg. Am. 85-A:1030-1039, 2003). Splice variants of the LMP protein exist; LMP-1 is the predominant form produced in human bone (Bunger et al., Calcif. Tissue Int. 73: 4446-54, 2003). LMP-3 is a LMP-1 mRNA splice variant with C-terminal truncation of the LIM domains, and like LMP-1, is effective in stimulating osteoblast differentiation in vitro, and stimulating ectopic bone formation in vivo (see Pola et al., Gene Ther. 11:683-693, 2004). However, LMP-1 is the predominant form expressed in human bone (see Bunger et al., Calcif. Tissue Int. 73:446-454, 2003). An exemplary LMP-1 sequence is set forth in GENBANK® Accession Nos. NM_{—}005451 and NM_{—}005451.3, which are incorporated by reference herein.

Mammal: This term includes both human and non-human mammals. Similarly, the term “subject” includes both human and veterinary subjects.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand.” Sequences on a nucleic acid sequence which are located 5′ to sequence of interest are referred to as “upstream sequences;” sequences a nucleotide sequence which are located 3′ to the sequence of interest are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together, such as in a wild-type gene. This includes nucleic acid vectors comprising an amplified or assembled nucleic acid which can be used to transform a suitable host cell. In one example, a recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. A host cell that includes the recombinant nucleic acid is referred to as a “recombinant host cell.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence. Thus, the two sequences are complementary.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

ORF (open reading frame): A series of nucleotide triplets (codons) coding for amino acids without any termination codons. These sequences are usually translatable into a peptide.

Peptide: A chain of amino acids of between 3 and 30 amino acids in length. In one embodiment, a peptide is from about 10 to about 25 amino acids in length. In yet another embodiment, a peptide is from about 11 to about 20 amino acids in length. In yet another embodiment, a peptide is about 10 amino acids in length. For example, a “Cox-2 peptide” is a series of contiguous amino acid residues from a Cox-2 protein.

Peptide modifications: The polypeptides disclosed herein include synthetic embodiments of peptides, such as Cox-2, FGF-2 or LIM-1. In addition, analogs (non-peptide organic molecules), derivatives (chemically functionalized peptide molecules obtained starting with the disclosed peptide sequences) and variants (homologs) of these proteins can be utilized in the methods described herein. Each polypeptide is comprised of a sequence of amino acids, which may be either L- and/or D-amino acids, naturally occurring and otherwise.

Peptides may be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into peptides to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of polypeptide. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press, Buffalo Grove, Ill., pp. 165-174 and Principles of Pharmacology Munson (ed.) 1995, Ch. 102, for descriptions of techniques used in CADD. Also included are mimetics prepared using such techniques.

Pharmaceutical agent: A chemical compound or composition capable of inducing a desired therapeutic or prophylactic effect when properly administered to a subject or a cell. “Incubating” includes a sufficient amount of time for a drug to interact with a cell.

A “therapeutically effective amount” is a quantity of a specific substance sufficient to achieve a desired effect in a subject being treated. For instance, this can be the amount necessary to induce fracture healing or to decrease a sign or symptom of the fracture in the subject. When administered to a subject, a dosage will generally be used that will achieve target tissue concentrations (for example, in bone) that has been shown to achieve a desired effect.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition, 1975, describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Biodegradable and biocompatible polymer scaffolds may be used as carriers for gene delivery (see Jang et al., Expert Rev. Medical Devices 1:127-138, 2004). These scaffolds usually contain a mixtures of one or more biodegradable polymers, for example and without limitation, saturated aliphatic polyesters, such as poly(lactic acid) (PLA), poly(glycolic acid), or poly(lactic-co-glycolide) (PLGA) copolymers, unsaturated linear polyesters, such as polypropylene fumarate (PPF), or microorganism produced aliphatic polyesters, such as polyhydroxyalkanoates (PHA), (see Rezwan et al., Biomaterials 27:3413-3431, 2006; Laurencin et al., Clin. Orthopaed. Rel. Res. 447:221-236). By varying the proportion of the various components, polymeric scaffolds of different mechanical properties are obtained. A commonly used scaffold contains a ratio of PLA to PGA is 75:25, but this ratio may change depending upon the specific application. Other commonly used scaffolds include surface bioeroding polymers, such as poly(anhydrides), such as trimellitylimidoglycine (TMA-gly) or pyromellitylimidoalanine (PMA-ala), or poly(phosphazenes), such as high molecular weight poly(organophasphazenes) (P[PHOS]), and bioactive ceramics. An advantage of these polymeric carriers is that they represent not only a scaffold but also a drug or gene delivery system.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (for example, a cDNA) independent of other sequences. The nucleotides can be ribonucleotides, deoxyribonucleotides, or modified forms of either nucleotide. The term includes single- and double-stranded forms of DNA. A Cox-2 polynucleotide is a nucleic acid encoding a Cox-2 polypeptide.

Polypeptide: Any chain of amino acids, regardless of length or post-translational modification (such as glycosylation or phosphorylation). In one embodiment, the polypeptide is Cox-2 polypeptide. A “residue” refers to an amino acid or amino acid mimetic incorporated in a polypeptide by an amide bond or amide bond mimetic, the “position” of the residue indicates its place in the amino acid sequence. A polypeptide has an amino terminal (N-terminal) end and a carboxy terminal end.

Probes and primers: A probe comprises an isolated nucleic acid attached to a detectable label or reporter molecule. Primers are short nucleic acids, and can be DNA oligonucleotides 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides.

Promoter: A promoter is an array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Both constitutive and inducible promoters are included (see e.g., Bitter et al., Methods in Enzymology 153:516-544, 1987).

Specific, non-limiting examples of promoters include promoters derived from the genome of mammalian cells (for example, a metallothionein promoter) or from mammalian viruses (for example, the retrovirus long terminal repeat; the adenovirus late promoter; the vaccinia virus 7.5K promoter). Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Protein purification: The Cox-2, FGF-2 and LIM-1 polypeptides and disclosed herein can be purified by any of the means known in the art. See, e.g., Guide to Protein Purification, ed. Deutscher, Meth. Enzymol. 185, Academic Press, San Diego, 1990; and Scopes, Protein Purification: Principles and Practice, Springer Verlag, New York, 1982. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Purified: The term purified does not require absolute purity; rather, it is intended as a relative term. Thus, for example, a purified peptide or polynucleotide preparation is one in which the peptide or polynucleotide is more enriched than the peptide or polynucleotide is in its natural environment within a cell. In one embodiment, a preparation is purified such that the protein or polynucleotide represents at least 50% of the total peptide or polynucleotide content of the preparation.

Retrovirus: Any virus in the family Retroviridae. These viruses have similar characteristics, specifically they share a replicative strategy. This strategy includes as essential steps reverse transcription of the virion RNA into linear double-stranded DNA, and the subsequent integration of this DNA into the genome of the cell. All native retroviruses contain three major coding domains with information for virion proteins: gag, pol and env. In one embodiment, a retrovirus is an avian sarcoma and leukosis virus, a mammalian B-type virus, a Murine leukemia-related virus, a Human T-cell leukemia-bovine leukemia virus, a D-type virus, a lentivirus, or a spumavirus. In another embodiment, the virus is a Rous sarcoma virus, a mouse mammary tumor virus, a human T-cell leukemia virus, a Mason-Pzifer monkey virus, a human immunodeficiency virus, a human foamy virus, or a Molony Leukemia Virus (MLV). A native retrovirus generally contains three genes known as “gag,” “pol,” and “env.” A replication defective retrovirus does not contain genetic sequences coding for these three retroviral genes: gag, pol and env.

Selectively hybridize: Hybridization under moderately or highly stringent conditions that exclude non-related nucleotide sequences.

In nucleic acid hybridization reactions, the conditions used to achieve a particular level of stringency will vary, depending on the nature of the nucleic acids being hybridized. For example, the length, degree of complementarity, nucleotide sequence composition (such as GC versus AT content), and nucleic acid type (such as RNA versus DNA) of the hybridizing regions of the nucleic acids can be considered in selecting hybridization conditions. An additional consideration is whether one of the nucleic acids is immobilized, for example, on a filter.

A specific, non-limiting example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., Vol. 1-3, ed. Sambrook et al., Cold Spring Harbor Laboratory Press, Cold Spring Harbor, N.Y., 1989). Washing can be carried out using only one of these conditions, e.g. high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a Cox-2 polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a Cox-2 polypeptide (or BMP or FGF) are typically characterized by possession of at least about 75%, for example at least about 80%, 90%, 95%, 96%, 97%, 98% or 99% sequence identity counted over the full length alignment with the amino acid sequence of Cox-2 using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Another indicia of sequence similarity between two nucleic acids is the ability to hybridize. The more similar are the sequences of the two nucleic acids, the more stringent the conditions at which they will hybridize. Substantially similar or substantially identical nucleic acids (and to subsequences thereof), such as Cox-2 and FGF-2 nucleic acids, include nucleic acids that hybridize under stringent conditions to any of these reference polynucleotide sequences. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, 2001, N.Y.; Tijssen, Hybridization With Nucleic Acid Probes, Part I: Theory and Nucleic Acid Preparation, Laboratory Techniques in Biochemistry and Molecular Biology, Elsevier Science Ltd., NY, 1993, and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” can be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid.

In nucleic acid hybridization reactions, the conditions used to achieve a particular level of stringency will vary depending on the nature of the nucleic acids being hybridized. The length, degree of complementarity, nucleotide sequence composition (e.g., GC v. AT content), and nucleic acid type (e.g., RNA versus DNA) of the hybridizing regions of the nucleic acids all influence the selection of appropriate hybridization conditions. Additionally, whether one of the nucleic acids is immobilized, for example, on a filter can impact the conditions required to achieve the desired stringency.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, or each of the conditions can be used, such as for 10-15 minutes each, in the order listed above, repeating any or all of the steps listed. However, as mentioned above, optimal conditions will vary, depending on the particular hybridization reaction involved, and can be determined empirically.

Spinal fusion: A technique in which one or more of the vertebra of the spine are united together (”fused“) so that motion is severely limited or no longer occurs between the vertebra. Spinal fusion can be preformed for the treatment of a fractured (broken) vertebra, the correction of deformity (spinal curves such as scoliosis or slippages such as spondylolisthesis), the elimination of pain from painful motion, the treatment of instability, and the treatment of some cervical disc herniations.

Stabilization and De-stabilizing Element: The stability of an mRNA can be measured by the half life of the mRNA under specific physiological conditions. Sequences within the 3′ untranslated region (UTR) of mRNAs have been shown to be important for message stability. Decreased message stability can be a result of increased degradation of the mRNA, and can result in decreased translation of the coding region of the mRNA.

De-stabilizing elements are specific nucleic acid sequences in the 3′ untranslated region of a gene that decrease the half-life of an mRNA. Adenine and Uridine-rich elements (AREs) are known to affect the stability of an mRNA (see Cok and Morrison, J. Biol. Chem. 276: 23179-85, 2001). One example of a de-stabilizing element is the sequence AUUUA.

Subject: Living multi-cellular vertebrate organisms, a category that includes both human and veterinary subjects, including human and non-human mammals.

Therapeutically effective amount: A quantity of a specific substance sufficient to achieve a desired effect in a subject being treated. For instance, this can be the amount necessary to accelerate fracture healing. When administered to a subject, a dosage will generally be used that will achieve target tissue concentrations (for example, in bone) that has been shown to achieve a desired in vitro effect.

Transduced: A transduced cell is a cell into which has been introduced a nucleic acid molecule by molecular biology techniques. As used herein, the term transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Transposon: Mobile elements that transpose gene segments in the genome. Tc1-like transposons, including sleeping beauty, are members of a superfamily of eukaryotic transposons that transpose in a “cut-and-paste” manner that requires the binding of an element-encoded enzyme, the transposase, to short inverted repeat/direct repeat (IR/DR) sequences flanking the element (see Plasterk, Curr. Top. Microbiol. Immunol. 204:125-143, 1996). Most of these elements integrate stably into TA-based target sites, which are duplicated upon insertion. Accordingly, the Tc1-like transposase has the unique ability to catalyze the excision of the DNA region flanked by the transposon elements in a plasmid DNA and promote its integration into the genome at TA target dinucleotide sites. Thus, this transposon system can be engineered into a gene transfer plasmid vector system that leads to stable integration with relative site-specificity (such as in TA dinucleotide sites).

The Tc1-like transposable elements in vertebrates are defective due to accumulation of mutations caused by a process known as “vertical inactivation” and are not functional (see Vos et al., Genes Dev. 10:755-761, 1996). However, Ivies and coworkers (see Cell 91:501-510, 1997) have reconstructed or “resurrected” a Tc1-like transposase from fish by correcting key mutations and referred to this molecularly reconstructed, functional fish Tc1-like transposon system as the “Sleeping Beauty” transposon system. This system has been shown to effectively transpose exogenous extrachromosomal DNA (supercoiled plasmid DNA) into genomic loci of human and mouse embryonic cells (see Ivies, et al., Cell 91:501-510, 1997, Luo et al., Proc. Natl. Acad. Sci. USA 95:10769-10773, 1998).

The first generation Sleeping Beauty plasmid vector system contained two plasmids: one plasmid expressed the reconstructed Tc1-like Sleeping Beauty transpoase, and the other plasmid contained the Tc1-like transposon IR/DR elements flanking the transgene-of-interest. A single plasmid including both elements (the transposase and the IR/DR elements), which is a “Sleeping Beauty” Tc1-like transposon-based vector is referred to as the Prince Charming (pPC) vector system (Harris et al., Anal. Biochem. 310:15-26, 2002, incorporated herein by reference).

Upregulated or increase: When used in reference to the expression of a nucleic acid molecule, such as a gene, or to an amount of a protein or other molecule, “increase” refers to any process which results in an increase in production of a molecule of interest.

An upregulation or an increase includes a detectable increase in the amount of a molecule in a sample. In certain examples, the amount is increased by at least 2-fold, for example at least 3-fold or at least 4-fold, as compared to a control (such an amount of present in an untreated cell).

Untranslated Region (UTR): A region of an mRNA that is not translated into a polypeptide. A 3′ untranslated region generally follows a stop codon in an mRNA sequence, and thus is downstream of the coding sequence of an mRNA. A 5′ untranslated sequence generally precedes the AUG (start) codon of an mRNA, and thus is upstream of the coding sequence of an mRNA. A “truncated” untranslated region is an untranslated region that is shorter than the untranslated region found in mRNA in a wildtype cell.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. Vectors can be viral vectors, such as adenoviral, retroviral, or lentiviral vectors. Vectors can be non-viral vectors, such as Sleeping Beauty plasmids or Prince Charming plasmids.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Cox-2 Nucleic Acid Constructs

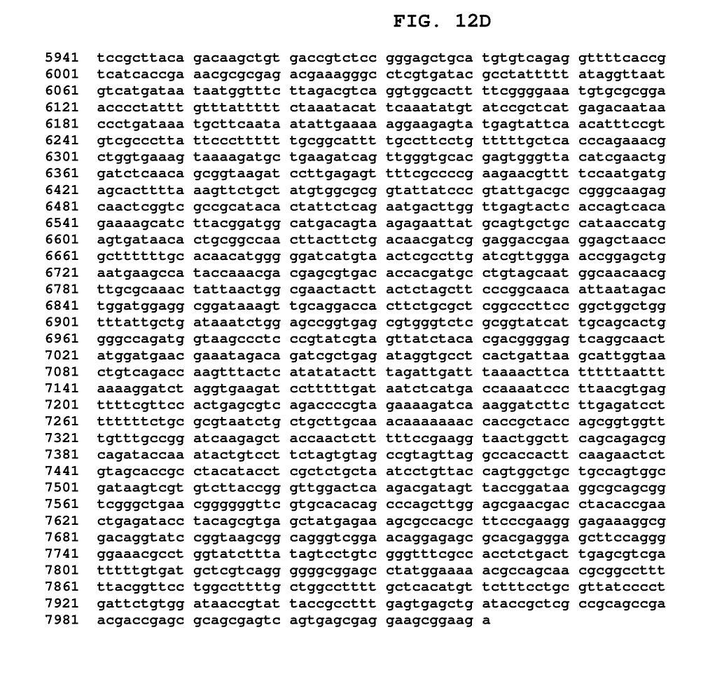

Nucleic acids encoding human Cox-2 are utilized in the methods disclosed herein. The sequence of human Cox-2 polypeptides, and nucleic acids encoding these polypeptides, are known in the art. An exemplary nucleic acid encoding human Cox-2, and the encoded amino acid sequence of human Cox-2, is shown in GENBANK® Accession No. M90100, which is incorporated herein by reference. A second human Cox-2 sequence with single nucleotide polymorphisms (SNPs) and short length polymorphisms demonstrating more than 98% sequence identity is set forth as GENBANK® Accession N. NM_{—}000963, which is also incorporated herein by reference. An exemplary nucleic acid sequence encoding human Cox-2, and an exemplary amino acid sequence of human Cox-2, are shown in FIG. 12 (see also SEQ ID NOs: 2 and 3).

In one non-limiting example, a human Cox-2 protein has the amino acid sequence set forth as:

(SEQ ID NO: 16)
MVARALLLCAVLALSHTANPCCSHPCQNRGVCMSVGFDQYKCDCTR
TGFYGENCSTPEFLTRIKLFLKPTPNTVHYILTHFKGFWNVVNNIP
FLRNAIMSYVLTSRSHLIDSPPTYNADYGYKSWEAFSNLSYYTRAL
PPVPDDCPTPLGVKGKKQLPDSNEIVGKLLLRRKFIPDPQGSNMMF
AFFAQHFTHQFFKTDHKRGPAFTNGLGHGVDLNHIYGETLARQRKL
RLFKDGKMKYQIIDGEMYPPTVKDTQAEMIYPPQVPEHLRFAVGQE
VFGLVPGLMMYATIWLREHNRVCDVLKQEHPEWGDEQLFQTSRLIL
IGETIKIVIEDYVQHLSGYHFKLKFDPELLFNKQFQYQNRIAAEFN
TLYHWHPLLPDTFQIHDQKYNYQQFIYNNSILLEHGITQFVESFTR
QIAGRVAGGRNVPPAVQKVSQASIDQSRQMKYQSFNEYRKRFMLKP
YESFEELTGEKEMSAELEALYGDIDAVELYPALLVEKPRPDAIFGE
TMVEVGAPFSLKGLMGNVICSPAYWKPSTFGGEVGFQIINTASIQS
LICNNVKGCPFTSFSVPDPELIKTVTINASSSRSGLDDINPTVLLK
ERSTEL

In additional embodiments, a human Cox-2 protein is at least 80%, at least 85%, at least 90%, at least 95% or at least 99% identical to SEQ ID NO: 16, wherein the polypeptides functions as a cyclooxygenase. In one specific, non limiting example, the second residue is a leucine. In other embodiments, a human Cox-2 protein includes at most 1, at most 2, at most 5, or at most 10 conservative amino acid substitutions in SEQ ID NO: 16 wherein the encoded protein functions as a cyclooxygenase.

SEQ ID NO: 16 is encoded by a polynucleotide set forth as GeneBank Accession No. M90100 (which is incorporated by reference herein) and degenerate variants of this nucleotide sequence (see GenBank Accession No. NM_{—}000963, incorporated herein by reference). Polynucleotides encoding human Cox-2, such as a protein at least 80%, at least 90% at least 99% identical to SEQ ID NO: 16 can readily be determined. Thus, in one example, nucleic acids used in the methods disclosed herein include a nucleic acid encoding SEQ ID NO: 16.

In addition to the above nucleic acid and amino acid sequences, nucleic acid and amino acid sequences that are substantially identical to these polynucleotide sequences can be used in the compositions and methods of the disclosure. Fore example, a substantially identical sequence can have one or a small number of deletions, additions and/or substitutions. Such nucleotide and/or amino acid changes can be contiguous or can be distributed at different positions within the nucleic acid or protein. A substantially identical sequence can, for example, have 1, or 2, or 3, or 4, or even more nucleotide or amino acid deletions, additions and/or substitutions, and encode a polypeptide that functions as a cyclooxygenase. Typically, the one or more deletions, additions and/or substitutions do not alter the reading frame encoded by a polynucleotide sequence, such that a modified (“mutant”) but substantially identical polypeptide is produced upon expression of the nucleic acid.

Naturally-occurring human Cox-2 mRNA includes a 5′ untranslated region upstream of the nucleotides encoding the Cox-2 protein, and a 3′ untranslated region downstream of the nucleotides encoding the Cox-2 protein. Generally the 5′ untranslated region is immediately upstream of the translation initiation codon, and the 3′ untranslated region is immediately downstream of the stop signal for translation. Wild-type Cox-2 is regulated at the translational level.

Wild-type human Cox-2 mRNA includes 12 AUUUA sequences in the 3′ untranslated region, which have been shown to destabilize mRNA. In the wild-type human Cox-2 gene, the AUUUA sequences reside in the 3′UTR and can be removed without disrupting the protein coding sequence upstream of the TGA stop codon (see Dixon et al., J. Biol. Chem. 275: 11750-57,2000, incorporated herein by reference).

In one non-limiting example, the 3′ untranslated region of GENBANK® Accession No. M90100 of wild-type Cox-2 includes nucleotides 1913-3387 of GENBANK® Accession No. M90100 and includes an AU-rich region, and includes multiple copies of AUUUA, which are known destabilizing elements. These nucleotides are set forth below.

(SEQ ID NO: 17)
aagucuaaugaucauauuuauuuauuuauaugaaccaugucuauua
auuuaauuauuuaauaauauuuauauuaaacuccuuauguuacuua
acaucuucuguaacagaagucaguacuccuguugcggagaaaggag
ucauacuugugaagacuuuuaugucacuacucuaaagauuuugcug
uugcuguuaaguuuggaaaacaguuuuuauucuguuuuauaaacca
gagagaaaugaguuuugacgucuuuuuacuugaauuucaacuuaua
uuauaaggacgaaaguaaagauguuugaauacuuaaacacuaucac
aagaugccaaaaugcugaaaguuuuuacacugucgauguuuccaau
gcaucuuccaugaugcauuagaaguaacuaauguuugaaauuuuaa
aguacuuuuggguauuuuucugucaucaaacaaaacagguaucagu
gcauuauuaaaugaauauuuaaauuagacauuaccaguaauuucau
gucuacuuuuuaaaaucagcaaugaaacaauaauuugaaauuucua
aauucauaggguagaaucaccuguaaaagcuuguuugauuucuuaa
aguuauuaaacuuguacauauaccaaaaagaagcugucuuggauuu
aaaucuguaaaaucagaugaaauuuuacuacaauugcuuguuaaaa
uauuuuauaagugauguuccuuuuucaccaagaguauaaaccuuuu
uagugugacuguuaaaacuuccuuuuaaaucaaaaugccaaauuua
uuaaggugguggagccacugcaguguuaucucaaaauaagaauauc
cuguugagauauuccagaaucuguuuauauggcugguaacauguaa
aaaccccauaaccccgccaaaagggguccuacccuugaacauaaag
caauaaccaaaggagaaaagcccaaauuauugguuccaaauuuagg
gugguuaaugaaguaccaagcugugcuugaauaacgauauguuuuc
ucagauuuucuguuguacaguuuaauuuagcaguccauaucacauu
gcaaaaguagcaaugaccucauaaaauaccucuucaaaaugcuuaa
auucauuucacacauuaauuuuaucucagucuugaagccaauucag
uaggugcauuggaaucaagccuggcuaccugcaugcuguuccuuuu
cuuuucuucuuuuagccauuuugcuaagagacacagucuucucaaa
cacuucguuucuccuauuuuguuuuacuaguuuuaagaucagaguu
cacuuucuuuggacucugccuauauuuucuuaccugaacuuuugca
aguuuucagguaaaccucagcucaggacugcuauuuagcuccucuu
aagaagauuaaaaaaaaaaaaaaag

In the sequence shown above, destabilizing elements, AUUUA are shown in bold.

In several embodiments, the 3′ untranslated region can be truncated to increase the stability of Cox-2 mRNA. Thus, in one embodiment, the nucleic acid encoding Cox-2 has a decreased number of nucleotides in the 3′ untranslated region as compared to a wild-type Cox-2 mRNA, or as compared to SEQ I D NO: 22. Thus, in several embodiments, the AU-rich region is deleted, for example, about 1000 to about 2000, or about 1000, about 1500 or about 2000 nucleotides of the AU-rich region are deleted. In additional embodiments, the region from about nucleotide 1900 to about nucleotide 3400, such as from about nucleotide 1900 to about nucleotide 3300 is deleted of the wild-type human Cox-2 nucleic acid sequence. Thus, in one example, SEQ ID NO: 17 is not present in the nucleic acid.

In one embodiment, a 3′ untranslated region can be truncated such that the stability of the mRNA is increased as compared to the stability of the mRNA in a wild-type cell. Thus, in several examples, degradation of the mRNA is reduced. Without being bound by theory, the half-life of the mRNA is increased.

In one example, at least one destabilizing element is removed. In another example, the 3′ untranslated region (UTR) is at most about 25 nucleotides in length, such as at most about 25, at most about 20, at most about 15, at most about 10 or at most about 5 nucleotides in length. Thus, the , the 3′ untranslated region (UTR) can be at most about 25 nucleotides, such as at most about 25, at most about 20, at most about 15, at most about 10 or at most about 5 consecutive nucleotides of SEQ ID NO: 17. In additional examples, the 3′ untranslated region does not include one copy or multiple copies of the sequence AUUUA in the transcribed mRNA, or includes reduced numbers of the sequence AUUUA as compared to a wild-type mRNA. For example, no copies of the sequence AUUUA can be included in the transcribed mRNA, at most one copy of AUUUA in the transcribed mRNA, at most two copies, at most three copies of AUUUA can be included in the 3′ UTR of the transcribed mRNA. In further examples one to three, such as two copies of AUUUA can be included in the transcribed mRNA. In an additional example, the 3′ UTR does not include any copies of AUUUA in the transcribed mRNA.

The 5′ untranslated region (UTR) of GENBANK® Accession No. M90100 is nucleotides 1-97: guccaggaacuccucagcagcgccuccuucagcuccacagccagacgcccucagacagcaaagcc uacccccgcgccgcgcccugcccgccgcugcg (SEQ ID NO: 18), which is followed by the start signal, “aug.” In some embodiments, this 5′ untranslated region is replaced by an optimized Kozak sequence.

The optimized Kozak sequence used in many of the embodiments of this disclosure is X₁CCX₂CCAUGG (SEQ ID NO: 15,wherein X₁ and X₂ can be any base). As a result of studying the conditions required for optimal translational efficiency of expressed mammalian genes, the ‘Kozak’ consensus sequence has been identified. It has been proposed that this defined translational initiating sequence (⁻⁶GCCA/GCC [symbol] G⁻⁴, SEQ ID NO: 15 wherein X₁ is a G and X₂ is an A or a G) can be included in vertebrate mRNAs located around the initiator codon to enhance translation. Efficient translation is obtained when an optimized Kozak sequence is utilized. Optimized Kozak sequences include those wherein the −3 position contains a purine base or, in the absence of a purine base, a guanine is positioned at +4. An additional example of an optimized Kozak sequence is UCCACCAUGG (SEQ ID NO: 15, wherein X₁ is a U and X₂ is an A). Another example of an optimized Kozak sequence is a nucleotide sequence comprising ACCAUGG, such as GCCACC [symbol] G (SEQ ID NO: 15, wherein X₁ is a G and X₂ is an A), where the A in the bolded AUG start codon is coordinate 1 and the A at position −3 could also be a G. A purine (usually A) in position −3 results in efficient initiation of translation, and in its absence, a G at position +4 results in efficient initiation.

Thus, in several embodiments the nucleic acid sequence includes an optimized Kozak sequence, such as (⁻⁶GCCA/GCC [symbol] G⁺⁴) (SEQ ID NO: 15, wherein X₁ is G and X₂ is A or G). Optimized Kozak sequences are known in the art; efficient translation is obtained when an optimized Kozak sequence is utilized. Optimized Kozak sequences include those wherein the −3 position contains a purine base or, in the absence of a purine base, a guanine is positioned at +4. One example of an optimized Kozak sequence is UCCACCAUGG (SEQ ID NO: 15, wherein X₁ is T and X₂ is A). Another example of an optimized Kozak sequence is a nucleotide sequence comprising ACCAUGG (nucleotides 3-9 of SEQ ID NO: 15), such as GCCACCAUGG (SEQ ID NO: 15), where the A in the underlined AUG start codon is coordinate 1 and the A at position −3 could also be a G. A purine (usually A) in position −3 produces efficient initiation of translation, and in its absence, a G at position +4 is produces efficient initiation. The T's in a DNA Kozak sequences are replaced by U's in corresponding mRNAs. Generally, the optimized Kozak sequence is operably linked to a nucleic acid encoding a protein of interest, such as but not limited to a Cox-2 protein, such as a human Cox-2 protein.

A heterologous promoter can be included in the construct. The promoter can be any promoter of interest, including constitutive and inducible promoters. In one embodiment, the promoter is a viral promoter. Other promoters include osteoblast gene specific promoters, housekeeping gene promoters (GAPDH, Actin, Cyclophilin), or chimeric promoters with viral enhancers with gene promoters, osteoblast enhancers with housekeeping gene or viral promoters. However, in other embodiments, the promoter is the Cox2 promoter. Generally, the promoter is operably linked to a nucleic acid encoding the protein of interest, such as but not limited to a Cox-2 protein, such as a human Cox-2 protein.

Polynucleotide sequences encoding Cox-2 (and/or FGF, BMP or LMP1) can be expressed in vitro by DNA transfer into a suitable host cell. Methods of stable transfer, meaning that the foreign DNA is continuously maintained in the host, are known in the art.

Polynucleotide sequences encoding a Cox-2 (and/or FGF, BMP or LMP1)can be inserted into an expression vector, such as a plasmid, virus or other vehicle known in the art that has been manipulated by insertion or incorporation of the Cox-2 (and/or FGF, BMP or LMP1) sequence. Polynucleotide sequences which encode Cox-2 (and/or FGF, BMP or LMP1) can be operatively linked to expression control sequences. In one embodiment, an expression control sequence operatively linked to a coding sequence is ligated such that expression of the coding sequence is achieved under conditions compatible with the expression control sequences.

The polynucleotide encoding Cox-2 (and/or FGF, BMP or LIM1) can be inserted into an expression vector that contains a promoter sequence which facilitates the efficient transcription of the inserted genetic sequence by the host. The expression vector typically contains an origin of replication, a promoter, as well as specific genes that allow phenotypic selection of the transformed cells. In one example, the expression control sequences include an optimized Kozak sequence, as disclosed above. Vectors suitable for use include, but are not limited to the T7-based expression vector for expression in bacteria (Rosenberg et al., Gene 56:125, 1987), the pMSXND expression vector for expression in mammalian cells (see Lee and Nathans, J. Biol. Chem. 263:3521, 1988) and baculovirus-derived vectors for expression in insect cells, and viral vectors. The DNA segment can be present in the vector operably linked to regulatory elements, for example, a promoter (such as the T7, metallothionein I, or polyhedron promoters). In one example, the vector is a viral vector, such as a retroviral vector or an adenoviral vector. Suitable vectors are known in the art, and include viral vectors such as retroviral, lentiviral and adenoviral vectors.

DNA or RNA viral vectors include an attenuated or defective DNA or RNA viruses, including, but not limited to, herpes simplex virus (HSV), papillomavirus, Epstein Barr virus (EBV), adenovirus, adeno-associated virus (AAV), Moloney leukemia virus (MLV) and human immunodeficiency virus (HIV) and the like. Defective viruses, that entirely or almost entirely lack viral genes, are preferred, as defective virus is not infective after introduction into a cell.

Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al. Mol. Cell. Neurosci., 2:320-330, 1991), defective herpes virus vector lacking a glycoprotein L gene (See Patent Publication RD 371005 A), or other defective herpes virus vectors (See PCT Publication No. WO 94/21807; and PCT Publication No.WO 92/05263); an attenuated adenovirus vector, such as the vector described by Stratford-Perricaudet et al. (J. Clin. Invest., 90:626-630 1992; La Salle et al., Science 259:988-990, 1993); and a defective adeno-associated virus vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Genes can also be introduced in a retroviral vector (e.g., as described in U.S. Pat. Nos. 5,399,346, 4,650,764, 4,980,289 and 5,124,263; all of which are herein incorporated by reference; Mann et al., Cell 33:153, 1983; Markowitz et al., J. Virol., 62:1120, 1988; PCT Application No. PCT/US95/14575; European Patent Application No. EP 453242; European Patent Application No. EP178220; Bernstein et al. Genet. Eng., 7:235, 1985; McCormick, BioTechnol., 3:689, 1985; PCT Publication No.WO 95/07358; and Kuo et al. Blood 82:845, 1993). Most retroviruses are integrating viruses that infect dividing cells. The lentiviruses are integrating viruses that infect nondividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. The gag, pol and env genes are coexpressed in the packaging cell line. These vectors can be constructed from different types of retrovirus, such as, HIV, MoMuLV (“murine Moloney leukemia virus” MSV (“murine Moloney sarcoma virus”); RSV (“Rous sarcoma virus”). In some examples, in order to construct recombinant retroviruses containing a nucleic acid sequence, a plasmid is constructed that contains the LTRs, the encapsidation sequence and the construct of the present disclosure comprising a nuclear targeting signal and a coding sequence. This construct is used to transfect a packaging cell line, which is able to supply the retroviral functions that are deficient in the plasmid. In general, the packaging cell lines are thus able to express the gag, pol and env genes. Such packaging cell lines have been described in the prior art, in particular the cell line PA317 (U.S. Pat. No. 4,861,719, herein incorporated by reference), the PsiCRIP cell line (See, WO90/02806), and the GP+envAm-12 cell line (See, WO89/07150). In addition, the recombinant retroviral vectors can contain modifications within the LTRs for suppressing transcriptional activity as well as extensive encapsidation sequences that can include a part of the gag gene (Bender et al., J. Virol., 61:1639, 1987). Recombinant retroviral vectors are purified by standard techniques.

Suitable vectors also include non-viral vectors. Exemplary non-viral vectors contain the sleeping beauty Tc1-like transposon and a DNA targeting sequence (DTS) facilitating nuclear entry. The cellular uptake (transfection) of non-viral vectors by mammalian cells can be improved on by using electroporation, insertion of a plasmid encased in liposomes, or microinjection. The use of DTS will promote nuclear uptake of the plasmid vector. An exemplary DTS is set forth below:

(SEQ ID NO: 1)
atgctttgcatacttctgcctgctggggagcctggggactttccac
accctaactgacacacattccacagctggttggtacctgca

One nonviral vector with the single plasmid “Sleeping Beauty” transposon-based vector is described Harris et al., (Anal. Biochem.310:15-26, 2002, incorporated herein by reference). A second exemplary non-viral vector contains the sleeping beauty transposon a nuclear entry sequence (DTS) from the SV40 enhancer and other tissue specific DTSs (see Dean et al., Gene Ther. 12:881-890, 2005, incorporated herein by reference). Naturally occurring DNA sequences in the promoters of viruses or in the promoters of mammalian genes can be used to achieve nuclear entry of the DNA containing a transgene by incorporating these sequences into plasmid-expression vectors that can be expressed in a non-dividing cell. The techniques exploit the inherent nuclear entry properties of DNA sequences in gene promoters to transport DNA into the nucleus after the binding of endogenous cell-specific transcription factor proteins to specific DNA sequences.

For example, U.S. Pat. No. 5,827,705 discloses a plasmid DNA vector that incorporates a SV40 viral DNA sequence into a nucleic acid molecule to stimulate nuclear entry of the nucleic acid molecule into any mammalian cell nucleus (see also Dean, Exp. Cell. Res. 230:293 1997; Dean et al., Gene Ther. 12:881-890, 2005). In this system, the plasmid DNA containing the nuclear entry sequence (the SV40 DNA sequence) is introduced into the cytoplasm of the host cell, wherein proteins that coat bind the SV40 viral DNA in the plasmid and allow transport of the entire plasmid into the nucleus of the host cells. Nuclear entry can occur in non-dividing cells.

In one embodiment, the DNA of the plasmid vector or viral vector is targeted into the nuclei, wherein one or more transgenes (such as nucleic acids encoding Cox-2, FGF-2, BMP-2/4 and/or LIM-1) of the vector are expressed. In one embodiment, the plasmid integrates into the genome of the specific cell type. In this embodiment, the DNA vector further includes a molecule to direct integration into the cell genome. Such integration sequences are known in the art, and include, for example, the inverted terminal repeats of adeno-associated virus (ITRs), retroviral long terminal repeats (LTRs), other viral sequences shown to cause incorporation or integration of viral DNA into the genome of the host cell. These sequences can be included in a Sleeping Beauty Tc1-like transposon system (see Ivics et al., Cell 91:501-510, 1997; Harris et al., Anal. Biochem. 310:15-26, 2002; Izsvak et al., Molecular Therapy 9:147-156, 2004; Dean et al., Gene Ther. 12:881-890, 2005, which are incorporated herein by reference).

Non-viral vectors that can be utilized for nucleic acid based therapy as taught herein include the Sleeping Beauty (or Prince Charming) Tc1-transposon-based plasmid vectors with or without one or more copies of a DTS. The Sleeping Beauty transposon systems employed in the methods disclosed herein can at least include a Sleeping Beauty transposon and a source of a Sleeping Beauty transposase activity. By Sleeping Beauty transposon is meant a nucleic acid that is flanked at either end by inverted repeats which are recognized by an enzyme having Sleeping Beauty transposase activity. By “recognized” is meant that a Sleeping Beauty transposase is capable of binding to the inverted repeat and then integrating the transposon flanked by the inverted repeat into the genome of the target cell. Representative inverted repeats that may be found in the Sleeping Beauty transposons of the subject methods include those disclosed in PCT Publication No. WO 98/40510 and PCT Publication No. WO 99/25817. Of particular interest are inverted repeats that are recognized by a transposase that shares at least about 80% amino acid identity to SEQ ID NO:01 of PCT Publication No. WO 99/25817. For a complete description of the Sleeping Beauty Transposon system, see U.S. Pat. No. 6,613,752, which is incorporated herein by reference.

Electroporation can be used to introduce nonviral vectors into cells and tissues in vivo. Generally, in this method, a high concentration of vector DNA is added to a suspension of host cell and the mixture is subjected to an electrical field of approximately 200 to 600 V/cm. Following electroporation, transformed cells are identified by growth on appropriate medium containing a selective agent. Electroporation has also been effectively used in animals or humans (see Lohr et al., Cancer Res. 61:3281-3284, 2001; Nakano et al, Hum Gene Ther. 12:1289-1297, 2001; Kim et al., Gene Ther. 10:1216-1224, 2003; Dean et al. Gene Ther. 10:1608-1615, 2003; and Young et al., Gene Ther. 10:1465-1470, 2003).

Polynucleotide sequences encoding Cox-2 (and/or FGF, BMP or LMP1) can be expressed in either prokaryotes or eukaryotes. Hosts can include microbial, yeast, insect and mammalian organisms. Methods of expressing DNA sequences having eukaryotic or viral sequences in prokaryotes are well known in the art. For example, biologically functional viral and plasmid DNA vectors capable of expression and replication in a host are known in the art. Such vectors are used to incorporate a DNA sequence encoding Cox-2 (and/or FGF, BMP or LMP1). Transfection of a host cell with recombinant DNA may be carried out by conventional techniques and are well known to those skilled in the art. Where the host is prokaryotic, such as E. coli, competent cells which are capable of DNA uptake can be prepared from cells harvested after exponential growth phase and subsequent treatment by the CaC12 method using procedures well known in the art. Alternatively, MgCl₂ or RbCl can be used. Transformation can also be performed after forming a protoplast of the host cell if desired, or by electroporation.

When the host is a eukaryote, methods of transfection of DNA as calcium phosphate co-precipitates, conventional mechanical procedures such as microinjection, electroporation, insertion of a plasmid encased in liposomes, or viral vectors may be used. Eukaryotic cells can also be cotransformed with a second foreign DNA molecule encoding a selectable phenotype, such as the herpes simplex thymidine kinase gene. Another method is to use a eukaryotic viral vector, such as simian virus 40 (SV40), murine leukemia virus, or bovine papilloma virus, to transiently infect or transform eukaryotic cells and express the protein (see for example, Eukaryotic Viral Vectors, Cold Spring Harbor Laboratory, Gluzman ed., 1982).

Nucleic acid based therapies for the treatment of bone fractures and for spinal fusion are disclosed herein. Such therapy would achieve its therapeutic effect by introduction of a therapeutically effective amount of a polynucleotide encoding Cox-2 into cells of the subject having the fracture. Delivery of the therapeutic polynucleotide can be achieved using a recombinant expression vector such as a chimeric virus or a colloidal dispersion system, or targeted liposomes.

Various viral vectors which can be utilized for nucleic acid based therapy as taught herein include adenovirus or adeno-associated virus, herpes virus, vaccinia, or an RNA virus such as a retrovirus (including HVJ, see Kotani et al., Curr. Gene Ther. 4:183-194, 2004). In one embodiment, the retroviral vector is a derivative of a murine or avian retrovirus, or a human or primate lentivirus. Examples of retroviral vectors in which a foreign gene can be inserted include, but are not limited to: Moloney murine leukemia virus (MoMLV), Harvey murine sarcoma virus (HaMuSV), murine mammary tumor virus (MuMTV), and Rous Sarcoma Virus (RSV). In one embodiment, when the subject is a human, a vector such as the gibbon ape leukemia virus (GaLV) can be utilized. A pseudotyped retroviral vector can be utilized that includes a heterologous envelope gene.

A number of additional retroviral vectors can incorporate multiple genes. All of these vectors can transfer or incorporate a gene for a selectable marker so that transduced cells can be identified and generated. By inserting a nucleic acid encoding Cox-2 (and/or FGF, BMP or LMP1) into the viral vector, along with another gene which can serve as viral envelope protein and also can encode the ligand for a receptor on a specific target cell, for example, the vector is now target specific. Retroviral vectors can be made target specific by modifications of the envelope protein by attaching, for example, a sugar, a glycolipid, or a protein. In one specific, non-limiting example, targeting is accomplished by using an antibody to target the retroviral vector.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the long terminal repeat (LTR). These plasmids are missing a nucleotide sequence which enables the packaging mechanism to recognize an RNA transcript for encapsidation. Helper cell lines which have deletions of the packaging signal include, but are not limited to ψ2, PA317, and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for a polynucleotide encoding Cox-2 (and/or FGF, BMP or LMP1) is a colloidal dispersion system. Colloidal dispersion systems include macromolecule complexes, nanocapsules, microspheres, beads, and lipid-based systems including oil-in-water emulsions, micelles, mixed micelles, and liposomes. One colloidal dispersion system is a liposome. Liposomes are artificial membrane vesicles which are useful as delivery vehicles in vitro and in vivo. It has been shown that large unilamellar vesicles (LUV), which range in size from 0.2-4.0 microns, can encapsulate a substantial percentage of an aqueous buffer containing large macromolecules. RNA, DNA and intact virions can be encapsulated within the aqueous interior and be delivered to cells in a biologically active form (Fraley et al., Trends Biochem. Sci. 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the nucleic acid of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al., Biotechniques 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include, for example, phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

Another targeting delivery system is the use of biodegradable and biocompatible polymer scaffolds (see Jang et al., Expert Rev. Medical Devices 1:127-138, 2004). These scaffolds usually contain a mixtures of one or more biodegradable polymers, for example and without limitation, saturated aliphatic polyesters, such as poly(lactic acid) (PLA), poly(glycolic acid), or poly(lactic-co-glycolide) (PLGA) copolymers, unsaturated linear polyesters, such as polypropylene fumarate (PPF), or microorganism produced aliphatic polyesters, such as polyhydroxyalkanoates (PHA), (see Rezwan et al., Biomaterials 27:3413-3431, 2006; Laurencin et al., Clin. Orthopaed. Rel. Res. 447:221-236). By varying the proportion of the various components, polymeric scaffolds of different mechanical properties are obtained. A commonly used scaffold contains a ratio of PLA to PGA is 75:25, but this ratio may change depending upon the specific application. Other commonly used scaffolds include surface bioeroding polymers, such as poly(anhydrides), such as trimellitylimidoglycine (TMA-gly) or pyromellitylimidoalanine (PMA-ala), or poly(phosphazenes), such as high molecular weight poly(organophasphazenes) (P[PHOS]), and bioactive ceramics. The gradual biodegradation of these scaffolds allows the gradual release of drugs or gene from the scaffold. Thus, an advantage of these polymeric carriers is that they represent not only a scaffold but also a drug or gene delivery system. This system is applicable to the delivery of plasmid DNA and also applicable to viral vectors, such as AAV or retroviral vectors, as well as transposon-based vectors.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

### Additional Nucleic Acid Constructs

The compositions and methods described herein can be used to express additional osteogenic growth factors, such as an FGF or an analog thereof, to promote fracture healing or spinal fusion. Thus, a nucleic acid encoding Cox-2 can be used alone, or in conjunction with a nucleic acid encoding an additional growth factor, such as, but not limited to, an FGF or LIM mineralization protein (LMP)1. Additional bone growth factors that can be delivered in combination with Cox-2 include parathyroid hormone, insulin-like growth factors, platelet derived growth factor, growth hormone and transforming growth factor-beta, and Lim Mineralization Protein 1.

Nucleic acids that encode therapeutic transgenes suitable for administration for promoting bone growth in humans and other mammals. The nucleic acids are delivery vehicles that encode osteogenic growth hormones that are capable of promoting stem cell renewal, increasing bone growth and enhancing angiogenesis. One such growth factor is fibroblast growth factor-2 (FGF-2). However, nucleic acids encoding any fibroblast growth factor can be utilized in the methods disclosed herein. Nucleic acids encoding LMP-1 are also of use in the methods disclosed herein.

Exemplary nucleotide and amino acid sequences of human FGF-2 are represented by SEQ ID NO: 19 and SEQ ID NO: 20, respectively (See also GENBANK® Accession No. M27968, incorporated herein by reference, and GENBANK® Accession No. AAA52488, incorporated herein by reference). An exemplary nucleic acid encoding FGF-2 is shown below:

(SEQ ID NO: 19)
atggcagccgggagcatcaccacgctgcccgccttgcccgaggatg
gcggcagcggcgccttcccgcccggccacttcaaggaccccaagcg
gctgtactgcaaaaacgggggcttcttcctgcgcatccaccccgac
ggccgagttgacggggtccgggagaagagcgaccctcacatcaagc
tacaacttcaagcagaagagagaggagttgtgtctatcaaaggagt
gtgtgctaaccgttacctggctatgaaggaagatggaagattactg
gcttctaaatgtgttacggatgagtgtttcttttttgaacgattgg
aatctaataactacaatacttaccggtcaaggaaatacaccagttg
gtatgtggcactgaaacgaactgggcagtataaacttggatccaaa
acaggacctgggcagaaagctatactttttcttccaatgtctgcta
agagctga,

which encodes:

(SEQ ID NO: 20)
MAAGSITTLPALPEDGGSGAFPPGHFKDPKRLYCKNGGFFLRIHPD
GRVDGVREKSDPHIKLQLQAEERGVVSIKGVCANRYLAMKEDGRLL
ASKCVTDECFFFERLESNNYNTYRSRKYTSWYVALKRTGQYKLGSK
TGPGQKAILFLPMSAKS

While the compositions and methods are described with respect to the human FGF-2 homolog, which is particularly suited for administration to human subjects, the compositions and methods disclosed herein are equally applicable to other mammalian FGF-2 orthologs, which can be selected by one of skill to correspond to the subject to which the nucleic acid, protein or cell is to be administered. Thus, for example, if the subject is a domestic livestock animal, such as a cow, a pig or a sheep, the FGF-2 nucleic acid can be selected from nucleic acids represented by GENBANK® Accession Nos: AX085265, AJ577089, and NM_{—}001009769, respectively, which are all incorporated herein by reference. Similarly, suitable FGF-2 homologs can be selected, and analogs produced, corresponding to any species of interest.

Exemplary analogs include modified FGF-2 nucleic acids and proteins that have been modified to include a signal peptide that promotes secretion of the translated FGF-2 product. In some examples, the nucleic acid encoding an additional growth factor includes a secretion signal. For example, a secretion signal sequence of use is a hybrid BMP2/4 secretion signal sequence which facilitates secretion of the translated product.

Nucleotide and amino acid sequences of an exemplary FGF-2 analog are provided in

SEQ ID NO:21 and SEQ ID NO:22, respectively.

(SEQ ID NO: 21)
atggtggccgggacccgctgtcttctagcgttgctgcttccccagg
tcctcctgggcggcgcggctggcctcgttccggagctgggccgcag
gaagttcgcggcggcgtcgtcgggccgcccctcatcccagccctct
gacgaggtcctgagcgagttcgagttgcggctgctcagcatgttcg
gcctgaaacagagacccacccccagcagggacgccgtggtgccccc
ctacatgctagacctgtatcgcaggcactcaggtcagccgggctca
cccgccccagaccaccggttggagagggcagccagccgagccaaca
ctgtgcgcagcttccaccatgaagaatctttggaagaactaccaga
aacgagtgggaaaacaacccggagattcttctttaatttaagttct
atccccacggaggagtttatcacctcagcagagcttcaggttttcc
gagaacagatgcaagatgctttaggaaacaatagcagtttccatca
ccgaattaatatttatgaaatcataaaacctgcaacagccaactcg
aaattccccgtgaccagacttttggacaccaggttggtgaatcaga
atgcaagcaggtgggaaagttttgatgtcacccccgctgtgatgcg
gtggactgcacagggacacgccaaccatggattcgtggtggaagtg
gcccacttggaggagaaacaaggtgtctccaagagacatgttagga
taagcaggtctttgcaccaagatgaacacagctggtcacagataag
gccattgctagtaacttttggccatgatggccggggccatgccttg
acccgacgccggagggccaagcgtgcagccgggagcatcaccacgc
tgcccgccttgcccgaggatggcggcagcggcgccttcccgcccgg
ccacttcaaggaccccaagcggctgtactgcaaaaacgggggcttc
ttcctgcgcatccaccccgacggccgagttgacggggtccgggaga
agagcgaccctcacatcaagctacaacttcaagcagaagagagagg
agttgtgtctatcaaaggagtgtctgctaaccgttacctggctatg
aaggaagatggaagattactggcttctaaaaatgttacggatgagt
gtttcttttttgaacgattggaatctaataactacaatacttaccg
gtcaaggaaatacaccagttggtatgtggcactgaaacgaactggg
cagtataaacttggatccaaaacaggacctgggcagaaagctatac
tttttcttccaatgtctgctaagagc,

which encodes:

(SEQ ID NO: 22)
MVAGTRCLLALLLPQVLLGGAAGLVPELGRRKFAAASSGRPSSQPS
DEVLSEFELRLLSMFGLKQRPTPSRDAVVPPYMLDLYRRHSGQPGS
PAPDHRLERAASRANTVRSFHHEESLEELPETSGKTTRRFFFNLSS
IPTEEFITSAELQVFREQMQDALGNNSSFHHRINIYEIIKPATANS
KFPVTRLLDTRLVNQNASRWESFDVTPAVMRWTAQGHANHGFVVEV
AHLEEKQGVSKRHVRISRSLHQDEHSWSQIRPLLVTFGHDGRGHAL
TRRRRAKRAAGSITTLPALPEDGGSGAFPPGHFKDPKRLYCKNGGF
FLRIHPDGRVDGVREKSDPHIKLQLQAEERGVVSIKGVSANRYLAM
KEDGRLLASKNVTDECFFFERLESNNYNTYRSRKYTSWYVALKRTG
QYKLGSKTGPGQKAILFLPMSAKS

A nucleotide sequence encoding an exemplary BMP2/4 secretion signal sequence is set forth below:

(SEQ ID NO: 23)
atggtggccgggacccgctgtcttctagcgttgctgcttccccagg
tcctcctgggcggcgcggctggcctcgttccggagctgggccgcag
gaagttcgcggcggcgtcgtcgggccgcccctcatcccagccctct
gacgaggtcctgagcgagttcgagttgcggctgctcagcatgttcg
gcctgaaacagagacccacccccagcagggacgccgtggtgccccc
ctacatgctagacctgtatcgcaggcactcaggtcagccgggctca
cccgccccagaccaccggttggagagggcagccagccgagccaaca
ctgtgcgcagcttccaccatgaagaatctttggaagaactaccaga
aacgagtgggaaaacaacccggagattcttctttaatttaagttct
atccccacggaggagtttatcacctcagcagagcttcaggttttcc
gagaacagatgcaagatgctttaggaaacaatagcagtttccatca
ccgaattaatatttatgaaatcataaaacctgcaacagccaactcg
aaattccccgtgaccagacttttggacaccaggttggtgaatcaga
atgcaagcaggtgggaaagttttgatgtcacccccgctgtgatgcg
gtggactgcacagggacacgccaaccatggattcgtggtggaagtg
gcccacttggaggagaaacaaggtgtctccaagagacatgttagga
taagcaggtctttgcaccaagatgaacacagctggtcacagataag
gccattgctagtaacttttggccatgatggccggggccatgccttg
acccgacgccggagggccaagcgt,

which encodes the acid sequence of the BMP2/4 secretion signal sequence:

(SEQ ID NO: 24)
MVAGTRCLLALLLPQVLLGGAAGLVPELGRRKFAAASSGRPSSQPS
DEVLSEFELRLLSMFGLKQRPTPSRDAVVPPYMLDLYRRHSGQPGS
PAPDHRLERAASRANTVRSFHHEESLEELPETSGKTTRRFFFNLSS
IPTEEFITSAELQVFREQMQDALGNNSSFHHRINIYEIIKPATANS
KFPVTRLLDTRLVNQNASRWESFDVTPAVMRWTAQGHANHGFWEVA
HLEEKQGVSKRHVRISRSLHQDEHSWSQIRPLLVTFGHDGRGHALT
RRRRAKR.

An exemplary nucleic acid encoding LMP-1 is set forth below:

(SEQ ID NO: 36)
agaacactggcggccgatcccaacgaggctccctggagcccgacgc
agagcagcgccctggccgggccaagcaggagccggcatcatggatt
ccttcaaagtagtgctggaggggccagcaccttggggcttccggct
gcaagggggcaaggacttcaatgtgcccctctccatttcccggctc
actcctgggggcaaagcggcgcaggccggagtggccgtgggtgact
gggtgctgagcatcgatggcgagaatgcgggtagcctcacacacat
cgaagctcagaacaagatccgggcctgcggggagcgcctcagcctg
ggcctcagcagggcccagccggttcagagcaaaccgcagaaggcct
ccgcccccgccgcggaccctccgcggtacacctttgcacccagcgt
ctccctcaacaagacggcccggccctttggggcgcccccgcccgct
gacagcgccccgcagcagaatggacagccgctccgaccgctggtcc
cagatgccagcaagcagcggctgatggagaacacagaggactggcg
gccgcggccggggacaggccagtcgcgttccttccgcatccttgcc
cacctcacaggcaccgagttcatgcaagacccggatgaggagcacc
tgaagaaatcaagccaggtgcccaggacagaagccccagccccagc
ctcatctacaccccaggagccctggcctggccctaccgcccccagc
cctaccagccgcccgccctgggctgtggaccctgcgtttgccgagc
gctatgccccggacaaaacgagcacagtgctgacccggcacagcca
gccggccacgcccacgccgctgcagagccgcacctccattgtgcag
gcagctgccggaggggtgccaggagggggcagcaacaacggcaaga
ctcccgtgtgtcaccagtgccacaaggtcatccggggccgctacct
ggtggcgctgggccacgcgtaccacccggaggagtttgtgtgtagc
cagtgtgggaaggtcctggaagagggtggcttctttgaggagaagg
gcgccatcttctgcccaccatgctatgacgtgcgctatgcacccag
ctgtgccaagtgcaagaagaagattacaggcgagatcatgcacgcc
ctgaagatgacctggcacgtgcactgctttacctgtgctgcctgca
agacgcccatccggaacagggccttctacatggaggagggcgtgcc
ctattgcgagcgagactatgagaagatgtttggcacgaaatgccat
ggctgtgacttcaagatcgacgctggggaccgcttcctggaggccc
tgggcttcagctggcatgacacctgcttcgtctgtgcgatatgtca
gatcaacctggaaggaaagaccttctactccaagaaggacaggcct
ctctgcaagagccatgccttctctcatgtgtgagccccttctgccc
acagctgccgcggtggcccctagcctgaggggcctggagtcgtggc
cctgcatttctgggtagggctggcaatggttgccttaaccctggct
cctggcccgagcctggggctccctgggccctgccccacccacctta
tcctcccaccccactccctccaccaccacagcacaccggtgctggc
cacaccagccccctttcacctccagtgccacaataaacctgtaccc
agctgtg

An exemplary LMP-1 amino acid sequence is set forth below:

(SEQ ID NO: 37)
MDSFKVVLEGPAPWGFRLQGGKDFNVPLSISRLTPGGKAAQAGVAV
GDWVLSIDGENAGSLTHIEAQNKIRACGERLSLGLSRAQPVQSKPQ
KASAPAADPPRYTFAPSVSLNKTARPFGAPPPADSAPQQNGQPLRP
LVPDASKQRLMENTEDWRPRPGTGQSRSFRILAHLTGTEFMQDPDE
EHLKKSSQVPRTEAPAPASSTPQEPWPGPTAPSPTSRPPWAVDPAF
AERYAPDKTSTVLTRHSQPATPTPLQSRTSIVQAAAGGVPGGGSNN
GKTPVCHQCHKVIRGRYLVALGHAYHPEEFVCSQCGKVLEEGGFFE
EKGAIFCPPCYDVRYAPSCAKCKKKITGEIMHALKMTWHVHCFTCA
ACKTPIRNRAFYMEEGVPYCERDYEKMFGTKCHGCDFKIDAGDRFL
EALGFSWHDTCFVCAICQINLEGKTFYSKKDRPLCKSHAFSHV.

Exemplary sequences are also shown in GENBANK® Accession No. NM_{—}005451.3, which is incoporated by reference herein.

A method is also provided that includes administering a therapeutically effective amount of a vector comprising a nucleic acid encoding bone morphogenetic protein (BMP)-4 operably linked to a heterologous promoter. In several examples, the BMP-4 comprises a hybrid signal sequence, whereas the hybrid sequence signal sequence comprises the secretion signal sequence of BMP-2 and sixteen C-terminus amino acid residues of the BMP-4 secretion signal sequence, and wherein the hybrid secretion signal sequence enhances the efficiency of BMP-4 protein secretion. In some examples, the hybrid signal sequence comprises 16 amino acid residues of the BMP-4 sequence signal sequence and the secretion signal sequence of BMP-2. The vector can also include an optimized Kozak sequence, as described above. Vectors of use are disclosed in Rundle et al., Bone 32: 591-601, which is incorporated herein by reference.

A FGF-2 analog, BMP-4 or an LMP-1 analog can include one or more amino acid substitutions (or additions or deletions) that increase stability of the secreted protein, typically without altering its activity. For example, one or more cysteine residues (up to all four of the cysteine residues) of FGF-2 can be modified. Typically, the second and third cysteines, such as the cysteines at positions 70 and 88 of FGF-2 (see U.S. Provisional Application No. 60/690,696, which is incorporated by reference herein in entirety) are mutated. For example, suitable mutations include cysteine to serine substitutions and cysteine to asparagine substitutions.

In addition to the above nucleic acid and amino acid sequences, nucleic acid and amino acid sequences that are substantially identical to these polynucleotide sequences can be used in the compositions and methods of the disclosure. Fore example, a substantially identical sequence can have one or a small number of deletions, additions and/or substitutions. Such nucleotide and/or amino acid changes can be contiguous or can be distributed at different positions within the nucleic acid or protein. A substantially identical sequence can, for example, have 1, or 2, or 3, or 4, or even more nucleotide or amino acid deletions, additions and/or substitutions. Typically, the one or more deletions, additions and/or substitutions do not alter the reading frame encoded by a polynucleotide sequence, such that a modified (“mutant”) but substantially identical polypeptide is produced upon expression of the nucleic acid.

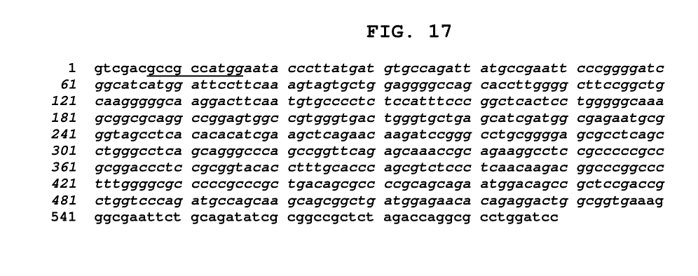

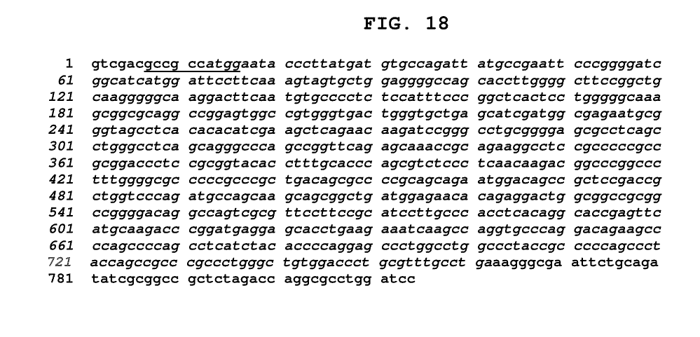

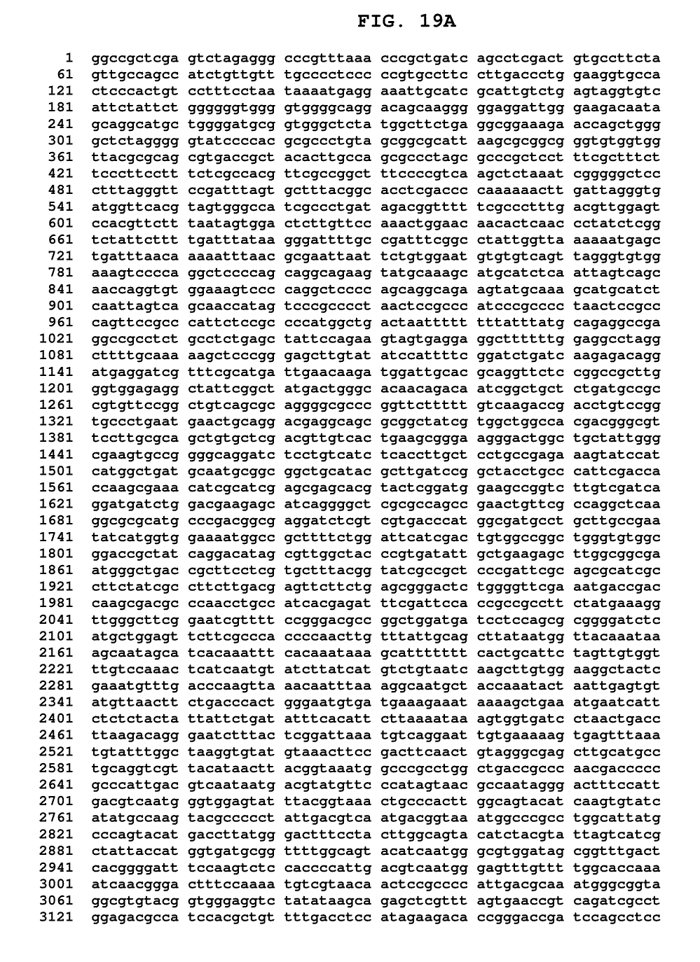

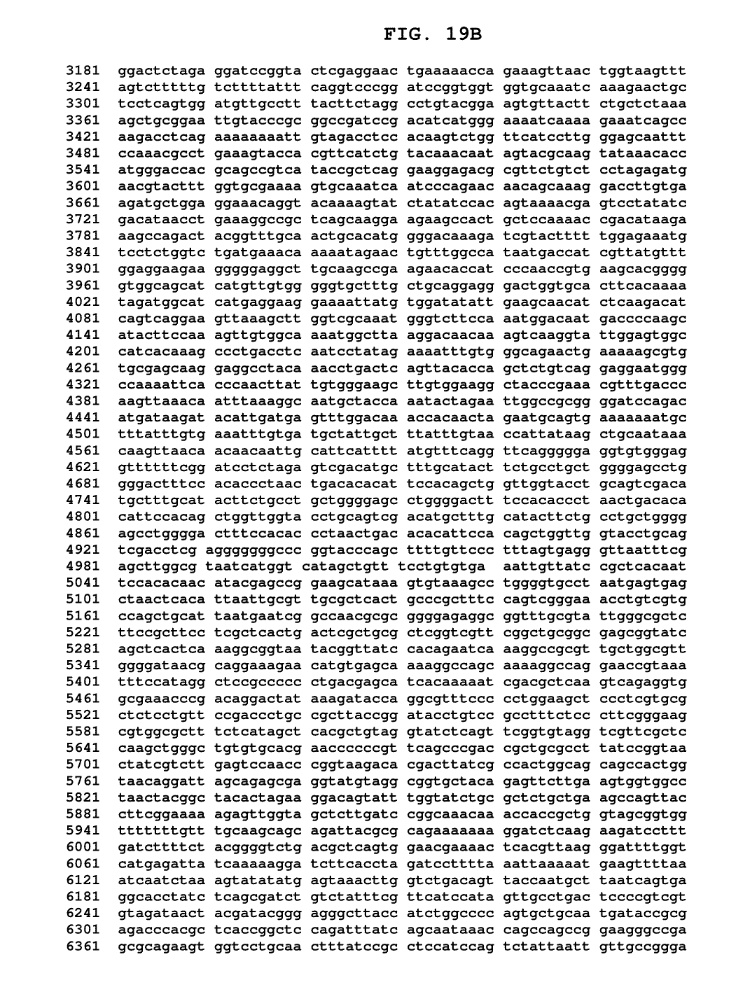

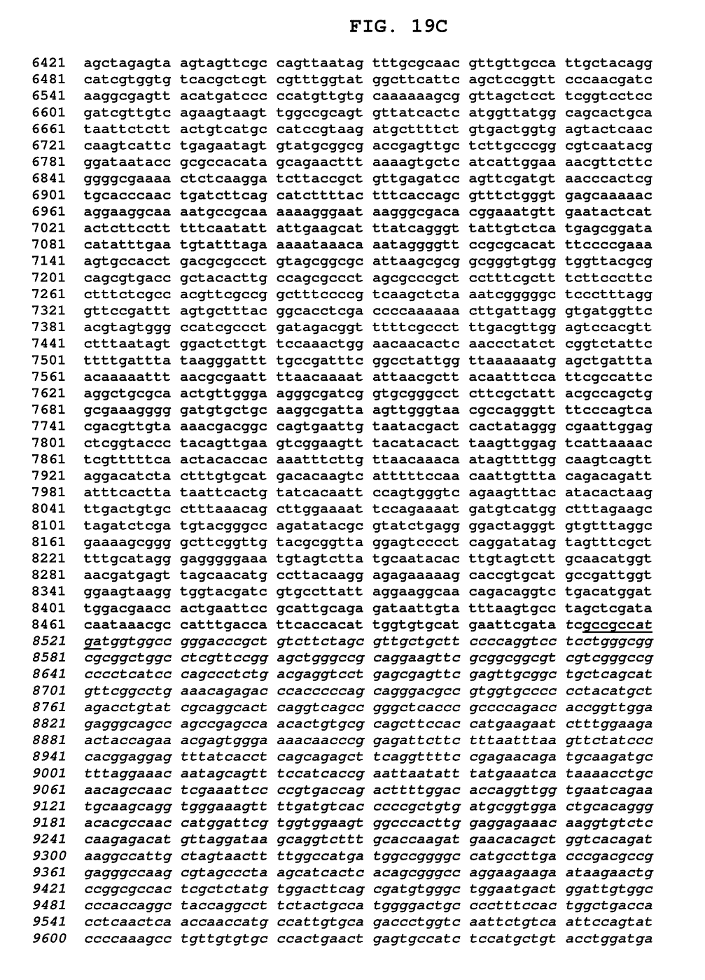

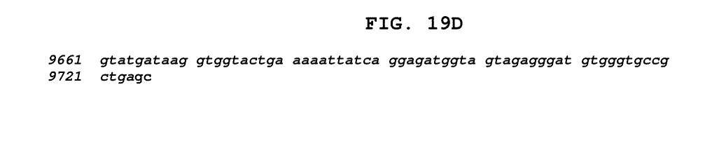

The similarity between polynucleotide and/or amino acid sequences is expressed in terms of the similarity between the sequences, otherwise referred to as sequence identity. Sequence identity is frequently measured in terms of percentage identity (or similarity); the higher the percentage, the more similar are the primary structures of the two sequences. Thus, a polynucleotide that encodes FGF or an FGF-2 analog (or another osteogenic growth factor) can be at least about 95%, or at least 96%, frequently at least 97%, 98%, or 99% identical to SEQ ID NO:21. Similarly, a polynucleotide that encodes LMP-1 or a LMP-1 analog can be at least about 95%, or at least 96%, frequently at least 97%, 98%, or 99% identical to SEQ ID NO:10 (see also FIGS. 16-18).

Thus, a sequence (that is a polynucleotide or polypeptide sequence) that is substantially identical, or substantially similar polynucleotide to a polynucleotide encoding FGF-2 (see above), BMP-4 or LMP-1 is encompassed within the present disclosure. Such polynucleotides can include insertions, deletions, and substitutions.

In additional examples, the nucleic acid encodes a protein at least 70% identical to an FGF-2 polypeptide, such as SEQ ID NO: 22. For example, at least 7 out of 10 nucleotides (or amino acids) within a window of comparison are identical to the reference sequence for FGF-2 (see above). Frequently, such sequences are at least about 80%, usually at least about 90%, and often at least about 95%, or more identical to a reference sequence. For example, the nucleic acid sequence encode a polypeptide at least 96%, 97%, 98% or even 99% identical to the reference sequence, such as an FGF-2 amino acid sequence set forth as SEQ ID NO: 22. Similarly, the nucleic acid can encode a protein at least 70% identical to a LMP-1 polypeptide. Such sequences can be least about 80%, usually at least about 90%, and often at least about 95%, or more identical to a reference sequence, such as at least 96%, 97%, 98% or even 99% identical to a LMP-1 amino acid sequence (see FIGS. 23-25).

As discussed above for Cox-2, the FGF-2 nucleic acid sequences, BMP or LMP-1 nucleic acid sequences can be inserted into a vector, such as a viral or a non-viral vector. Thus a nucleic acid encoding an FGF, such as FGF-2, or encoding a BMP, such as a BMP-4, or encoding LIM-1, can be operably linked to an optimized Kozak sequences, and inserted into a vector of interest, as described above for Cox-2. The optimized Kozak sequences and vectors of interest are disclosed above are also of use with a nucleic acid encoding FGF-2 and/or LMP-1. The vector can also include a hybrid signal sequence, whereas the hybrid sequence signal sequence comprises the secretion signal sequence of BMP-2 and sixteen C-terminus amino acid residues of the BMP-4 secretion signal sequence, and wherein the hybrid secretion signal sequence enhances the efficiency of protein secretion.

Methods are provided herein for increasing prostaglandin production in a host cell. A prostaglandin is any member of a group of lipid compounds that are derived enzymatically from fatty acid. Prostaglandins contain 20 carbon atoms, including a 5-carbon ring, and are well known in the art. The prostaglandins include prostaglandins (PG)D, PGE, PGF, PGH, and PGI. Prostaglandin E₂ (PGE2) is generated from the action of prostaglandin E synthases on prostaglandin H₂ (PGH2). In one example, a method is provided for increasing the production of PGE₂. Prostaglandins can be measured by a number of assays known in the art, including immunoassays. These immunoassays are commercially available, such as from Cisbio, Inc. and Cayman, Inc.

### Methods of Treatment

Methods are provided to promote fracture healing that utilize the vectors described herein. The fracture can be in any bone, including but not limited to cranial bones such as the frontal bone, parietal bone, temporal bone, occipital bone, sphenoid bone, ethmoid bone; facial bones such as the zygomatic bone, superior and inferior maxilla, nasal bone, mandible, palantine bone, lacrimal bone, vomer bone, the inferior nasal conchae; the bones of the ear, such as the malleus, incus, stapes; the hyoid bone; the bones of the shoulder, such as the clavicle or scapula; the bones of the thorax, such as the sternum or the ribs; the bones of the spinal column including the cervical vertebrae, lumbar vertebrae, and thoracic vertebrae; the bones of the arm, including the humerus, ulna and radius; the bones of the hands, including the scaphoid, lunate, triquetrum bone, psiform bone, trapezium bone, trapezoid bone, cpitate bone, and hamate bone; the bones of the palm such as the metacarpal bones; the bones of the fingers such as the proximal, intermediate and distal phalanges the bones of the pelvis such as the ilium, sacrum and coccyx; the bones of the legs, such as the femur, tibia, patella, and fibulal; the bones of the feet, such as the calcaneus, talus, navicular bone, medial cuneiform bone, intermediate cuniform bone, lateral cuneiform bone, cuboidal bone, metatarsal bone, proximal phalanges, intermediate phalanges and the distal phalanges; and the pelvic bones. In one example, a bone fracture is repaired in the absence of extra-skeletal bone formation, such as in the absence of bone formation in the soft tissues.

Methods are also provided to promote spinal fusion using the vectors described herein. Spinal fusion can be induced in any of the vertebrae, including, but not limited to, the cervical vertebrae, lumbar vertebrae, and thoracic vertebrae. In one example, spinal fusion occurs in the absence of extra-skeletal bone formation, such as in the absence of bone formation in the soft tissues.

In additional embodiments, the nucleic acids disclosed herein can be used to treat subjects that have a broken bone due to any disease, defect, or disorder which affects bone strength, function, and/or integrity, such as decreasing bone tensile strength and modulus. Examples of bone diseases include, but are not limited to, diseases of bone fragility, such as osteoporosis. Other examples include subject affected with malignancies and/or cancers of the bone such as a sarcoma, such as osteosarcoma. The methods can be used in human or non-human subjects (see for example, Akhter et al., Calcif. Tissue Int. 78: 357-362, 2006).

For administration to a subject a therapeutically effective dose of a pharmaceutical composition containing the nucleic acids encoding Cox-2 (and/or an FGF such as FGF2, a BMP such as BMP2/4 or mineralization protein such as LMP1) can be included in a pharmaceutically acceptable carrier. The pharmaceutical compositions are prepared and administered in dose units. Solid dose units are tablets, capsules, single injectables and even suppositories. For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administrations of subdivided doses at specific intervals.

The pharmaceutical compositions are in general administered topically, periosteally, intravenously, intramedullary, orally or parenterally or as implants, but even rectal use is possible in principle. In one embodiment, administration is local, such as to the perioseum. In another embodiment, administration is local such as by intramedullary injection. Intramedullary administration can be achieved by direct injection into the marrow space of the fracture site, without injection into the periosteum or bone cortex. Intramedulary administration can be administered by direct injection into the marrow, or by insertion of a K-wire through the intramedullary canal.

In one emobidment, a scaffold is utilized, which includes, for example, a combination of polylactic acid and glycolic acid. By varying the proportion of the two components is polymers of different mechanical properties are obtained. Thus, in several embodiments, the ratio of polylactic acid: glycolic acid is about 1:1, about 2:1, about 3:1 or about 4:1. In one example, the scaffolding includes about 75% polylactic acid and about 25% glycolic acid.

In another embodiment, the scaffold is porous. For example, a scaffold can be about 85%, about 90%, about 95%, about 98% porous, such as for non-weight bearing tissue. In additional examples, the scaffold is about 5% porous, about 10% porous, about 15% porous or about 20% porous, such as for weight bearing tissue. The porosity can be determined, for example, by the fusing of micro spheres with CO₂ treatment. In this process commercial pellets of the polymer are converted to microspheres of the desired size, which are fused to develop a porous structure. By altering the micropore size scaffolds of different microporosity can be obtained. These scaffolds can be used, for example, with plasmid DNA, AAV viral vectors, transposon vectors and MLV vectors. Additional scaffolds are described above.

Suitable solid or liquid pharmaceutical preparation forms are, for example, granules, powders, tablets, coated tablets, (micro)capsules, suppositories, syrups, emulsions, suspensions, creams, aerosols, drops or injectable solution in ampoule form and also preparations with protracted release of active compounds, in whose preparation excipients and additives and/or auxiliaries such as disintegrants, binders, coating agents, swelling agents, lubricants, flavorings, sweeteners, solubilizers or scaffolds are customarily used as described above. The pharmaceutical compositions are suitable for use in a variety of drug delivery systems. For a brief review of present methods for drug delivery, see Langer, Science 249:1527-1533, 1990.

The therapeutically effective amount of the pharmaceutical compositions can be administered locally or systemically. A therapeutically effective dose is the quantity of a nucleic acid encoding Cox-2 (and/or an FGF, BMP, LMP1) necessary to induce bone growth, increase the expression of prostaglandins, or to heal a fracture. The administration of the nucleic acid of Cox-2 (and/or FGF, BMP, or LMP1) can arrest the symptoms of the fracture or spinal disorder, such as pain, and its complications in the subject. Amounts effective for this use will, of course, depend on the severity of the affliction and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

In several embodiments, the nucleic acid encoding Cox-2 can be administered in a conjunction with an additional therapeutic agent. Thus, for the treatment of a fracture, the nucleic acid encoding Cox-2 can be administered in conjunction with LMP-1, FGF-2, a BMP or related proteins, or a nucleic acid encoding one or more of these proteins. For example, LMP-1 is a transcription regulator that has been shown to induce bone formation by recruiting multiple bone morphogenic proteins (BMPs) (see Liu et al., Bone 35:673-681, 2004). Without being bound by theory, expression of LMP-1, along with Cox-2, induces cells to produce osteoinductive paracrine factors, such as prostaglandins and BMPs, that further enhance osteoblast differentiation in surrounding cells. In other embodiments, the nucleic acid encoding Cox-2 can be administered in conjunction with a bone morphogenic protein, such as BMP-2, BMP-4, BMP-7, and/or BMP-2/4 hybrid, or a nucleic acid encoding a bone morphogenic protein. In another embodiment, the Cox-2 expressing nucleic acid can be administered in conjunction with a growth factor, such as FGF-2 to further enhance fracture repair. The nucleic acid encoding Cox-2 can also be submitted with a factor that promotes angiogenesis.

Other agents can also be administered, such as chemical compounds. In one embodiment, a nucleic acid encoding Cox-2 (or FGF-2, a BMP, or LMP-1) is administered with an anti-inflammatory agent, such as a non-steroidal anti-inflammatory agent. In another embodiment, a nucleic acid encoding Cox-2 is administered with an antibiotic, antifungal, or anti-viral agent. Thus, the nucleic acid encoding Cox-2 can be used alone or with other therapeutic agents, to promote fracture healing and/or spinal fusion.

### Screening Assays

A method is provided herein to identify therapeutically effective nucleic acid molecule that promotes fracture repair or spinal fusion. The method includes producing a transverse, midshaft femoral fracture by a three-point bending technique after a stabilizing K-wire is inserted in a non-human animal; and administering the nucleic acid molecule by intramedullary injection or percutaneous injection into the fracture. An increase in the healing of the transverse, midshaft femoral fracture as compared to a control indicates that the nucleic acid molecule promotes fracture repair or spinal fusion. In several examples, the nucleic acid molecule is administered into marrow cavity of the fracture site through a surgically placed catheter.

The nucleic acid molecule can be a vector comprising a recombinant nucleic acid encoding a protein operably linked to a heterologous promoter. The vector can be a viral vector, such as a retrovial vector, or can be a non-viral vector, such as a transposon-based non-viral vector. Optionally, the vector can include an optimized Kozak sequence.

It is disclosed herein agents can promote angiogenesis, stimulate bone formation, affect bone resorption, and fracture healing or spinal fusion. Thus, a method is provided to evaluate agents of interest in order to determine if they (1) promote angiogenesis, (2) stimulate bone formation, and (3) increase bone resorption. An agent that has all three properties will be of use in promoting fracture healing. In one embodiment, the agent of interest is a nucleic acid encoding a growth factor, cytokine, or enzyme. The agent can be any agent of interest, including but not limited to, nucleic acid constructs, proteins, pharmaceutical agents, growth factors, cytokines small molecules and organic compounds. In one example, the agent is a fibroblast growth factor or a cyclooxygenase.

Any assay known to one of skill in the art can be used to evaluate angiogenesis. For example Masson's trichrome stain for angiogensis (Wang et al., J. Orthop. Res. 23: 671-679, 2005); Intravital microscopy methods (Zhang et al., J. Trauma Injury Infect. Crit. Care 54: 979-985, 2002); μCT methods (Doschak et al., J. Anat. 203: 223-233, 2003); Immunohistochemical staining methods (Homer et al., Bone 19: 353-362, 1996; Lewinson et al., Histchem. Cell. Biol. 116: 381-388, 2001; Reed et al., J. Orthop. Res. 20: 593-599, 2002; Masaki et al., Circ. Res. 90: 966-973, 2002; Hayami et al., J. Rheumatol. 30: 2207-2217, 2003; Acikalin et al., Dig. Liver Dis. 37: 162-169, 2005); Dye perfusion methods (Doschak et al., J. Orthop. Res. 22: 942-948, 2004; Murata et al., Lab. Invest. 74: 68-77, 1996).

In addition, any assay can be use to determine if an agent stimulates bone formation. Non-limiting examples of suitable assays include radiographic methods (Lehmann et al., Bone 35: 1247-1255, 2004; Rundle et al., Bone 32: 591-601, 2003; Nakamura et al., J. Bone Miner. Res. 13: 942-949, 1998); microcomputed tomography (μCT) methods (Nakamura et al., J. Bone Miner. Res. 13: 942-949, 1998; Lehmann et al., Bone 35: 1247-1255, 2004; Tamasi et al., J. Bone Miner. Res. 18: 1605-1611, 2003; Shefelbine et al., Bone 36:480-488, 2005); peripheral quantitative computed tomographic methods (Rundle et al., Bone 32: 591-601, 2003; Tamasi et al., J. Bone Miner. Res. 18: 1605-1611, 2003); dual energy X-ray absorptiometry methods (Holzer et al., Clin. Orthop. Rel. Res. 366: 258-263, 1999; Nakamura et al., J. Bone Miner. Res. 13: 42-949, 1998); histomorphometry methods (Lehmann et al., Bone 35: 247-1255, 2004; Tamasi et al., J. Bone Miner. Res. 18:1605-1611, 2003; Li et al., J. Bone Miner. Res. 17: 791-799, 2002; Schmidmaier et al., Bone 30: 816-822; 2002; Nakamura et al., J. Bone Miner. Res. 13:942-949, 1998; Sheng et al., Bone 30: 486-491, 2002); Masson's trichrome stain for collagen (Rundle et al., Bone 32: 591-601, 2003); Goldner's stain for collagen (Holzer et al., Clin. Orthop. Rel. Res. 366: 258-263; 1999); Von Kossa's silver stain for bone (Schmidmaier et al., Bone 30: 816-822, 2002); Safranin Orange stain for collagen (Schmidmaier et al., Bone 30: 816-822, 2002); Toluidine Blue stain for cartilage (Holzer et al., Clin. Orthop. Rel. Res. 366: 258-263, 1999); Immunohistochemistry methods (Rundle et al., Bone 32: 591-601, 2003; Li et al., J. Bone Miner. Res. 17: 791-799, 2002; Safadi et al., J. Cell Physiol. 196: 51-62, 2003; Iwaki et al., J. Bone Miner. Res. 12: 96-102, 1997); Serum biochemical marker assays (Gundberg, Clin. Lab. Med. 20: 489-501, 2000); bone resportion gene expression (Bolander, Proc. Soc. Exp. Biol. Med. 200:165-170, 1992; Safadi et al., J. Cell Physiol. 196: 51-62, 2003; Rundle et al., Clin. Orthop. Rel. Res. 403: 253-263, 2002; Gerstenfeld et al., J. Orthop. Res. 21: 670-675, 2003).

Similarly, any assay known to those of skill in the art can be used to evaluate bone resorption. For example, radiographic methods (Lehmann et al., Bone 35: 1247-1255, 2004; Rundle et al., Bone 32: 591-601, 2003; Nakamura et al., J. Bone Miner. Res. 13: 942-949, 1998); μCT methods (Nakamura et al., J. Bone Miner. Res. 13: 942-949, 1998; Lehmann et al., Bone 35: 1247-1255, 2004; Tamasi et al., J. Bone Miner. Res. 18: 1605-1611, 2003; Shefelbine et al., Bone 36:480-488, 2005); peripheral quantitative computed tomographic methods (Rundle et al., Bone 32: 591-601, 2003; Tamasi et al., J. Bone Miner. Res. 18: 1605-1611, 2003); dual energy X-ray absorptiometry methods (Holzer et al., Clin. Orthop. Rel. Res. 366: 258-263, 1999; Nakamura et al., J. Bone Miner. Res. 13: 42-949, 1998); histomorphometry methods (Lehmann et al., Bone 35: 247-1255, 2004; Tamasi et al., J. Bone Miner. Res. 18:1605-1611, 2003; Li et al., J. Bone Miner. Res. 17: 791-799, 2002; Schmidmaier et al., Bone 30: 816-822; 2002; Nakamura et al., J. Bone Miner. Res. 13:942-949, 1998; Sheng et al., Bone 30: 486-491, 2002); Masson's trichrome stain for collagen (Rundle et al., Bone 32: 591-601, 2003); Goldner's stain for collagen (Holzer et al., Clin. Orthop. Rel. Res. 366: 258-263; 1999); Von Kossa's silver stain for bone (Schmidmaier et al., Bone 30: 816-822, 2002); Safranin Orange stain for collagen (Schmidmaier et al., Bone 30: 816-822, 2002); Toluidine Blue stain for cartilage (Holzer et al., Clin. Orthop. Rel. Res. 366: 258-263, 1999); Immunohistochemistry methods (Rundle et al., Bone 32: 591-601, 2003; Li et al., J. Bone Miner. Res. 17: 791-799, 2002; Safadi et al., J. Cell Physiol. 196: 51-62, 2003; Iwaki et al., J. Bone Miner. Res. 12: 96-102, 1997); Serum biochemical marker assays (Garnero and Delmas, Curr. Opin. Rheumatol. 16: 428-434, 2004; Robins, Curr. Opin. Clin. Nutri. Metab. Care 6: 65-71, 2003); bone formation gene expression (Bolander, Proc. Soc. Exp. Biol. Med. 200:165-170, 1992; Safadi et al., J. Cell Physiol. 196: 51-62, 2003; Rundle et al., Clin. Orthop. Rel. Res. 403: 253-263, 2002; Gerstenfeld et al., J. Orthop. Res. 21: 670-675, 2003).

Test compounds can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckemann et al., J. Med. Chem. 37: 2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Nad. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33.2059, 1994; Carell et al., Angew. Chem. Int. Ed. Engl. 33:2061, 1994; and Gallop et al., J. Med. Chem. 37:1233, 1994.

Libraries of compounds can be presented in solution (e.g., Houghten, Biotechniques 13:412-421, 1992), or on beads (Lam, Nature 354:82-84, 1991), chips (Fodor, Nature 364:555-556, 1993), bacteria or spores (U.S. Pat. No. 5,223,409), plasmids (Cull et al., Proc. Nad. Acad. Sci. USA 89:1865-1869, 1992) or on phage (Scott and Smith, Science 249:386-390, 1990; Devlin Science 249:404-406, 1990; Cwirla et al., Proc. Natl. Acad. Sci. USA 87:6378-6382, 1990; Felici, J. Mol. Biol. 222:301, 1991).

The disclosure is illustrated by the following non-limiting Examples.

### EXAMPLES

It is disclosed herein that in vivo gene therapy can be used to accelerate the repair of bone fractures. In vivo administration of an engineered viral vector to promote fracture healing represents a potentially highly efficient procedure for clinical applications. A VSV-G pseudotyped murine leukemia virus (MLV)-based retroviral vector was used to target transgene expression to the proliferating periosteal cells arising shortly after bone fracture.

The human Cox-2 transgene used in the studies disclosed herein was modified to improve mRNA stability and protein translation in that the 3′UTR is removed. The 3′UTR of the wild-type human Cox-2 gene contains several AU-rich domains (responsible for rapid mRNA degradation). These were deleted and the native translation signal was replaced with an optimized Kozak translation signal sequence.

In vitro studies with this MLV vector expressing the modified human Cox-2 transgene revealed increased and sustained Cox-2 gene expression over several passages in both osteoblasts and rat bone marrow stromal cells. In addition, PGE₂ production was increased in the transgenic cells as compared to the control cells. The transgenic stromal cells expressing the modified human Cox-2 gene also exhibited increased alkaline phosphatase compared to cells transduced with a control gene.

In vivo studies in the rat femur fracture model revealed bony union by 21 days after fracture in animal fractures treated with this MLV-human Cox-2 gene therapy. At this time the residual cartilage observed in the fracture gap of control animals had been replaced by bone in animals treated with the human Cox-2 transgene. The time to union had been accelerated by at least one week compared to control fractures. Moreover, no extraskeletal bone formation was seen with this Cox-2 gene therapy. Thus, the expression of an osteoinductive therapeutic transgene from a retroviral vector is an effective approach to accelerate bony union in a normally healing fracture model.

In vivo intramedullary injection of MLV-based vectors expressing a bone-growth promoting gene, such as a BMP2/4 hybrid gene, at the fracture site of the rat femur promoted bone formation but avoided ectopic bone formation at extra-skeletal muscles. In vivo intramedullay or percutaneous injection with MLV-based vectors expressing other bone formation promoting genes, such as a modified FGF-2 gene, or the LMP-1 gene, in the rat femur showed that this approach also led to a significant increase in the size of healing callus, suggesting an acceleration of fracture repair. These studies suggest that the Cox-2 therapy may be used in conjunction with a BMP, FGF-2, or LMP-1, and indicate this in vivo viral vector injection model of rat femoral fracture repair can be used as an in vivo screening test for fracture repair promoting genes, proteins, and/or small molecule therapeutic agents.

Examples presented within have demonstrated that other viral vectors, such as lentiviral vectors, or non-viral transposon-based vectors, such as the Sleeping Beauty Tc1-like transposon-based vectors are effective in delivering fracture repair promoting genes to the rat femoral fracture sites.

### Example 1

### Materials and Methods

Cell isolation and culture: Rat marrow stromal cells and calvarial osteoblasts were isolated from newborn Fischer 344 pups. Bone marrow cells were collected by flushing the diaphyses and marrow cavity with sterile DMEM medium (see Gysin et al., Gene Ther. 9:991-999, 2002). Calvarial osteoblasts were isolated as previously described (see Stringa et al., Endocrinology 136:3527-3533, 1995) and stored in liquid nitrogen until use. Cells were cultured in DMEM supplemented with 10% fetal bovine serum.

Construction of a MGF-based human Cox-2 retroviral vector: A human Cox-2 cDNA construct in a pcDNA 3 plasmid vector was obtained (see Hla and Neilson, Proc. Natl. Acad. Sci. USA 89:7384-7388, 1992). The full-length Cox-2 protein coding region was modified to minimize the 5′ and 3′ untranslated regions and to increase the efficiency of translation from the Kozak sequence. The enhanced Kozak sequence was XCCG/ACCATGG (SEQ ID NO: 15), which altered the second amino acid from leucine to valine. In addition, the 3′ untranslated sequence (UTR) was limited to 14 nucleotides and avoided the destabilizing AT-rich region. The modified insert was subcloned into the VR1012 vector to provide compatible restriction enzyme sites for subcloning into the pCLSA MFG-based retroviral vector. The Cox-2 vector was digested with Bam HI, the 5′ overhangs filled-in with the Klenow fragment of DNA polymerase I and the linear vector was than digested with Xba Ito release the 1910 by cDNA fragment. The cDNA fragment was gel-isolated and purified with Gene Clean (Bio 101, Vista, Calif.). The 4.9 kb VR1012 vector (see Hartikka et al., Human Gene Ther. 7:1205-1217, 1996) was digested with EcoRV and Xba I, gel-isolated and purified. The human Cox-2 cDNA and VR1012 vector were then ligated and transformed into Top10 competent cells (Invitrogen). DNA was isolated with a Qiagen Miniprep Spun column protocol from individual clones and the resulting DNA was sequenced to confirm its integrity. The 1835 nucleotide Cox-2 cDNA was removed from the VR1012 vector with Sal I and BamHI and ligated into an MFG-based vector, pCLSA, at compatible sites (see Peng et al., Mol Ther 4:95-104, 2001).

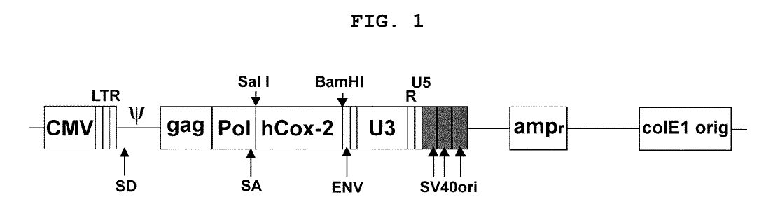

The structure of the MGF-like pCLSA-human (h)-Cox-2 retroviral expression vector is shown in FIG. 1. The sequence of the modified human Cox-2 gene was confirmed by DNA sequencing. The complete sequence is shown in the Sequence Listing section.

Retroviral vectors that expressed full length human LMP-1, HA (such as Tyr Pro Tyr Asp Val Pro Asp Tyr Ala (SEQ ID NO: 25))-tagged full length LMP-1 and HA-tagged truncated LMP-1. A cDNA coding sequence was prepared by PCR from the HA-tagged Enigma-pcDNA3 expression plasmid (see Wu and Gill, J. Biol. Chem. 269:25085-25090, 1994). A Sal I site and optimized Kosak sequence were introduced into the HA-tagged LMP cDNA sequence to facilitate cloning into the plasmid and retroviral vectors and to optimize protein expression with the primer pairs: 5′-gtcgacgccgccatggaatacccttatgatgtg-3′ (SEQ ID NO: 26) and 5′-ggctcacacatgagagaagg-3′(SEQ ID NO: 27). The PCR products were prepared with Native Pfu Polymerase (Stratagene) cloned into the pCR Blunt-II TOPO vector, was released with Sall and BamHI and cloned into the pCLSA vector for retrovirus production.

The cDNA coding sequence containing an optimized Kozak sequence for LMP-1 was prepared as a PCR product from the Enigma-pcDNA3 expression plasmid with the primer set 5′-gtcgacgccgccatggattccttcaagtagtg-3′ (SEQ ID NO: 28) and 5′-gaaatgcagggccacgactc-3′(SEQ ID NO: 29). An EcoRI fragment from the pCRII Topo Vector (Invitrogen) was cloned into the pCMV-AD vector (Clontech) to pick-up a 5′-SalI site and a 3′ BamHI site from the cloning vector. The LMP-1 cDNA was cut from the pCMV-AD vector with SalI and BamHI and subcloned into the SalI/BamHI sites of the pCLSA vector for retrovirus production.

Retrovirus production and transduction: pCLSA-Cox-2, pCLSA-LMP-1, pCLSA BMP-2/4, or pCLSA-BMPFGFC2SC3N DNA were each purified by column chromatography (Qiagen) prior to vector production. The retroviral vector was produced in 293T cells by transient transfection with calcium phosphate with three plasmids: 1) the MLV-Cox-2 (or MLV-LMP, MLV-BMP, or MLV-FGF) expression cassette vector described above, 2) the CMV driven VSV-G envelope expression vector and 3) the CMV driven Gag/Pol expression vector. Two harvests of virus were taken over 48-72 hours and the virus was concentrated by centrifugation. To determine the effectiveness of the virus preparation, HT1080 cells were transduced with virus at multiplicities of infection of 8-16.

The conditioned medium was collected for determination of prostaglandin E2 production (see below). The cells were washed with phosphate buffered saline (PBS), the media removed and the cell pellet was stored at −80 C prior to analysis of Cox-2 protein by Western immunoblotting (WIB) or assay of alkaline phosphatase (ALP) activity. The remainder of this virus was stored at −80° C. for transduction of rat marrow stromal cells or calvarial osteoblasts. Transfection efficiency with a parallel set of GFP-transduced cells was determined by FACS analysis. Transfection efficiencies ranged from 70% to 90%.

Lentivirus Production: The design of the lentiviral vectors used in studies described in this disclosure were based on third-generation self-inactivating lentiviral-based vectors. Accordingly, all of the lentivirus accessory proteins, the lentivirus envelope protein and the lentiviral TAT gene were deleted from the vector system. In addition, a deletion in the lentiviral 3′ LTR sequences that are essential for lentivirus expression was also deleted. These deletions ensure wild-type lentiviruses will not be generated during the vector production. The genes needed for lentiviral vector production were separated into four different expression vectors. The first plasmid vector, pHIV-9, contained the RNA transcript of the genomic RNA of viral vector and was able to be packed into the viral vector particle. This plasmid construct also contained the expression cassette for the transgene-of-interest expression (for example, BMP2/4). The second plasmid vector, pHIV-GP, contained the gene that was able to generate the viral gag and pol proteins, which are essential for viral vector assembling. The third plasmid vector, pCMV-Rev, was the vector containing the viral rev protein, which is essential for the gene expression of viral genomic RNA and HIV-GP gene expression. The fourth plasmid, pCMV-VSV-G, contained the envelope of the viral vector. The envelope gene was derived from the vascular stomatits virus. This allowed effective pseudotyping of lentiviral-based vectors to expand the cell type specificity of the viral vectors, including human and rodent bone and marrow stromal cells.

In general, lentiviral-based vectors were produced as follows. 293T cells were seeded at a density of 3×10⁶ cells in a 10-cm culture plate in Dulbecco Modified Eagle's medium (DMEM) with 10% fetal bovine serum (FBS). After the cells reached ˜70-80% confluency after 24 hrs in cultures, the cell medium was changed to fresh DMEM with 10% FBS. An hour later, the DNA mixtures of the four aforementioned plasmids (i.e., 20 μg of pHIV-9, 10 of pHIV-GP, 5 μg of pCMV-Rev and 1 μg of pCMV-VSV-G) were added to a solution of calcium chloride and appropriated amounts of phosphate buffer to form the DNA-calcium phosphate precipitates. Thirty minutes later, the DNA-calcium phosphate solution was added directly to the 10-cm plate of 293T cells to initiate the transduction. At six hours after the transduction, the cell medium was replaced with DMEM with 10% FBS and 5 mM of sodium butyrate. Most of the components for vector production (such as VSV-G and gag/pol genes) were toxic to the 293T host cells. The harvest of the viral vectors was done within 72 hr after the transduction. Accordingly, the viral vectors were harvested from the CM of the treated cells 24 hours after the transduction. The collected CM was filtered through a 0.45 μM filter membrane, aliquoted, and stored at −80° C. until use. The harvest of viral vectors continued every day for two additional days.

When a high titer viral stock was needed, the CM was concentrated by ultra-centrifugation (26,000×g for 90 min). The pellet was resuspended in 1/30 of original volume of PBS with 4% of lactose. When high titer viral stock was needed, the viral particles in CM were pelleted down by ultra-centrifugation (26,000×g for 90 min), and resuspended in phosphate buffered saline containing 4% lactose to ˜1/30 of the original volume before use.

Western Blot Analysis: Transduced cells (100,000) were lysed directly in 50-100 μl of SDS PAGE-sample buffer (4% SDS, 10% β-mercaptoethanol and 10 mM Tris (pH 8.0). Proteins were fractionated through a 12% polyacrylamide-SDS gel and transblotted onto a 0.2 μm PVDF membrane (BioRad, Hercules, Calif.). The membrane was blocked with 1% skim milk for 90 min and incubated for 90 min with 1 μg/ml polyclonal anti-human Cox-2 antibody (Cayman Chemical Company, Ann Arbor, Mich.). The blot was washed and then incubated for 90 min with 1:1000-diluted horse radish perioxidase (HRP)-labeled donkey anti-rabbit IgG. The blot was washed and incubated with a 1:10 dilution of SuperSignal West Pico chemiluminescent substrate (Pierce, Rockford, Ill.) for 3-5 minutes and exposed to Kodak X-Omat MR film. The amount of Cox-2 in cell extracts was compared to a known amount of ovine Cox-2 standard on the same blot. Replicate wells were counted to determine the number of cells expressing Cox-2.

Production of HA-tagged LMP-1 in HT1080 cells: HT1080 fibroblast cells were transduced with retrovirus as described above. The cellular production of HA-tagged LMP-1 protein was verified by western immunoblot analysis with the anti-HA monoclonal antibody, HA.11 (Covance Research Products).

PGE₂ Production Assay: The Prostaglandin E₂ Express EIA Kit from Cayman was used to assay PGE₂ levels in the conditioned media (CM). All procedures were performed according to the manufacturer's specifications. A standard curve was prepared using PGE2-spiked CM from nontransduced rat marrow stromal and osteoblasts to validate the assay under conditions used for transduced cells. Samples were assayed in triplicate.

ALP activity assay: Replicates of transduced (MLV-Cox-2 or MLV-GFP) rat osteoblasts and marrow stromal cells were plated in 10% FBS/DMEM and cultured to confluence. Media was removed, cells were rinsed with PBS and the cell layers were extracted in 1 ml of a solution of 0.01% Triton X-100 in a buffer containing 12.5 mmol/liter NaHCO₃, 12.5 mmol/liter Tris and 0.01% sodium azide at pH 7.0. ALP activity was quantitated in aliquots of the extracts by measuring the time-dependent hydrolysis of p-nitrophenyl phosphate as previously described (Kyeyune-Nyombi et al., Calcif. Tissue Int. 56:154-159, 1995).

Femoral Fracture Model: Transverse, midshaft femoral fractures were produced in 12-week-old male Fischer 344 rats (Harlan-Sprague Dawley, Indianapolis, Ind.) using the three-point bending technique described by Bonnerens and Einhorn (J. Orthop. Res. 2: 97-101, 1984). The movement of the animals was not restricted at any time after surgery.

### Retroviral Vector Delivery Into Fracture Site:

a. Single Lateral Percutaneous Injection: MLV-Cox-2 (or MLV-BMP2/4) was injected to the periosteum at one day post-fracture in a single percuraneous injection from the lateral aspect of the fracture site. With the animal under general anesthesia, a 29G needle was used to deliver 1 to 2 x 10⁷ transforming units of the vector in a total volume of 150 μl (Rundle et al., Bone 32:591-601, 2003). To maximize accuracy, the injection procedure was visualized using a fluoroscope (Fluoroscan). MLV-β-galactosidase was constructed as previously described (Peng, et al., Mol. Therapy 4:95-104, 2001) and used as a control gene. The fractures were allowed to heal for different post-fracture intervals prior to harvest for assessment of Cox-2 (or BMP2/4) transgene expression or radiological and histological assessment of fracture healing.

b. Catheter Delivery The femur was internally stabilized prior to fracture by the insertion and attachment of a 1.14 mm diameter Kirschner (K)-wire through the entire intramedullary canal. Two incisions were made at opposite ends of the femur, 0.5 cm in length over the greater trochanter and a 1 cm in length lateral to the knee and parallel to the axis of the leg. An additional incision of the muscle tissue separated it from the lateral patellar tendon. The patellar tendon was displaced medially, exposing the condyle, which was then drilled with a Dremel tool using a #52 drill bit. The diameter of the hole was just sufficient to insert the Kirschner-wire. The wire was pushed through the medullary canal and forced through the greater trochanter at the location of the first incision. The Kirschner-wire was withdrawn from the hole at the trochanter and replaced with a sterile 19 G thin wall stainless steel 304 tube also inserted in retrograde fashion. This tubing acted as a sleeve to guide a 20 G catheter that was inserted anterograde fashion into the medullary cavity. The tubing was removed during the catheter insertion. A 1.14 mm diameter K-wire was then inserted back through the hole at the trochanter immediately adjacent to the catheter. The end of the pin at the trochanter was then bent and the wire was pulled from the condyle end until the bent portion of the wire seated firmly at the trochanter yet did not obstruct the catheter. The catheter needle was removed at this point, leaving only the catheter tubing inside the bone. The wire was then cut flush with the condyle and the incisions closed with the appropriate sutures (#4 for muscle at the condyle and the patellar tendon and #3 for the skin). The wound was treated with antiseptic to minimize the risk of infection. The catheter was capped with a luer Lock cap which was removed for introduction of retroviral vectors. A 25 G spinal needle was used to deliver 1-2×10⁷ transforming units in a volume of 150 μl one day of fracture.

X -ray analysis of fracture healing: Fractured bone tissues were harvested for X-ray analysis at the indicated time after surgery. These intervals were chosen for the fracture tissues characteristic of 1) the switch from intramembranous bone formation to chondrogenesis (7 days), 2) chondrogenesis and endochondral bone formation (10 days) and 3) endochondral bone formation (21 days) (Bolander, Proc. Soc. Exp. Biol. Med. 200:165-170, 1992). Upon examination of the initial X-ray results, additional fractures were harvested at 17 days to more accurately establish the time at which Cox-2 gene expression altered normal fracture healing. Tissues from four animals that received the MLV-Cox-2 vector were compared to tissues from four animals that received the MLV-β-galactosidase vector. The femurs were examined for mineralized fracture tissues by X-ray (Faxitron, Wheeling, Ill.) and fixed in 10% neutral buffered formalin for evaluation of bone formation by histology.

Histological analysis of the fracture callus: Histological analysis of the fracture calluses harvested at 7, 10, 17 or 21 days after fracture was performed after fixation and X-ray analysis. Fracture tissues were demineralized in EDTA, embedded in paraffin and 5-micron sagittal sections of the fracture tissues adhered to SUPERFROST PLUS™ slides (Fisher, Pittsburgh, Pa.).

Immunohistochemistry was used to assess Cox-2 protein expression in fracture tissues expressing either the MLV-Cox-2 or the MLV-β-galactosidase gene. Tissue sections were rinsed with PBS and endogenous peroxidase was blocked with 3% H₂O₂ at room temperature for 15 minutes. The sections were rinsed with PBS and non-specific sites were blocked with 1:50 normal goat serum for 30 minutes at room temperature. The tissues were incubated with 1:300 anti-Cox-2 antibody at room temperature for 1 hour. Sections were rinsed and incubated with 1:200 diluted anti-rabbit IgG-biotin antibody (Pierce) for 15 minutes at room temperature. The slide was rinsed and a 1:200 dilution of Streptavidin-HRP (Vector Labs, Burlingame, Calif.) was added for 15 minutes at room temperature. DAB-H₂O₂ substrate solution was applied for 5 min and the tissue was rinsed with PBS. The polyclonal anti-Cox-2 antibody (#160107, Cayman) reacted with both human and rat Cox-2. All immunohistochemistry procedures were performed using the automated Ventana ES immunohistochemistry system (Ventana Medical Systems, Tucson, Ariz.), according to the manufacturer's specifications. All sections were examined using an Olympus BX-60 microscope (Olympus America, Melville, N.Y.) and photomicrographs obtained with a Sony camera (Sony America, NY, N.Y.).

To evaluate the effects of Cox-2-mediated healing, the fracture callus was examined for evidence of bone formation by Van Giesen staining. Cartilage formation was detected by Safranin Orange staining and osteoclasts by tartrate resistant acid phosphatase staining. Both were quantified from sagittal sections at the center of the fracture callus, as identified by the space made in the marrow cavity by the stabilizing Kirschner wire.

Real-time RT-PCR Analysis of Cox-2 transgene expression in the fracture callus: For the analysis of Cox-2 transgene expression, groups of four animals that received the MLV-Cox-2 transgene were compared with groups of four animals that received the MLV-β-galactosidase control gene at 4, 7, 14 and 21 days post-fracture. These times were three days prior to those groups of animals harvested for X-ray and histologic analysis, when the effects of gene expression would be expected to be observable in the fracture histology.

Immediately after euthanasia, the femur was dissected and the fracture callus separated from the metaphyses and epiphysis of the femur with a circular saw. The callus immediately transferred to liquid nitrogen. The fracture tissues were reduced to powder with a biopulvarizer (Biospec Products, Bartleville, Okla.) and the RNA purified by Trizol extraction, according to the manufacturer's specifications (Invitrogen, Grand Island, N.Y.). The RNA was further purified using RNEASY® columns (Qiagen, Valencia, Calif.). Precipitated total RNA was washed with 70% ethanol three times and the dry pellet was resuspended in RNAse-free water. Total RNA was quantified and purity was evaluated using a NANODROP™ spectrophotometer (Agilent Technologies, Mountain View, Calif.), and RNA integrity was confirmed using a BIOANALYZER™ (Agilent).

RNA was treated with DNAse I prior to cDNA preparation for 15 minutes at room temperature. cDNA was prepared from 1 μg of total RNA, 0.5 μg of oligo-dT₁₂₋₁₈ primer using the Superscript III reverse transcription kit (Invitrogen).

Rat and human Cox-2 cDNA abundance was quantified by real-time RT-PCR relative to a cyclophilin (a housekeeping gene). PCR primer sets (IDT, Coralville, Iowa) were developed using the most variable regions of the human and rat cDNA sequence to distinguish between endogenous rat Cox-2 and the human Cox-2 expressed by the retroviral vector (Table 1). Real-time PCR was performed for 30 cycles in an Opticon DNA Engine (MJ Research, Reno, Nev.). Each reaction was performed in a 25 μl solution containing 300 pmol of each primer, and 400 μmol of each deoxyribonucleotide. Amplification conditions were 95° C. for 6 minutes, primer annealing at 56.4° C. for human Cox-2 and cyclophilin or 64.0° C. for rat Cox-2 for 1 minutes, and 72° C. for 30 seconds. Fifty ng of plasmid DNA as a template to obtain a temperature gradient and select the optimum temperature of PCR product production for each primer set. The human Cox-2 PCR primers did not anneal to the rat Cox-2 template at this temperature, and distinguished human Cox-2 transgene from the endogenous rat Cox-2 gene during amplification. The number of molecules of Cox-2 mRNA was determined from a standard curve.

TABLE 1
Primers used for real-time PCR
measurements of gene expression.

| | | | | NCBI |
|---|---|---|---|---|
| Gene | | | Product | Database |
| (nucleotides) | Primer Sequences | Tm | Size | Accession # |
| Cyclophilin A | 5′gcatacaggtcctggcatct | 56.4 | 190 | BC059141 |
| (315-505) | 5′gctctcctgagctacagaag | | | |
| | (SEQ ID NO: 30 AND 31) | | | |
| Human Cox-2 | 5′ggttgctggtggtaggaatgt | 56.4 | 336 | M90100 |
| (1354-1690) | 5′ccagtaggcaggagaacatat | | | |
| | (SEQ ID NO: 32 AND 33) | | | |
| Rat Cox-2 | 5′aaggcctccattgaccagag | 64.8 | 445 | AF233596 |
| (1326-1871) | 5′cacttgcgttgatggtggct | | | |
| | (SEQ ID NO: 34 AND 35) | | | |

### Example 2

### Cox-2 Retroviral Vector and In Vitro Transduction

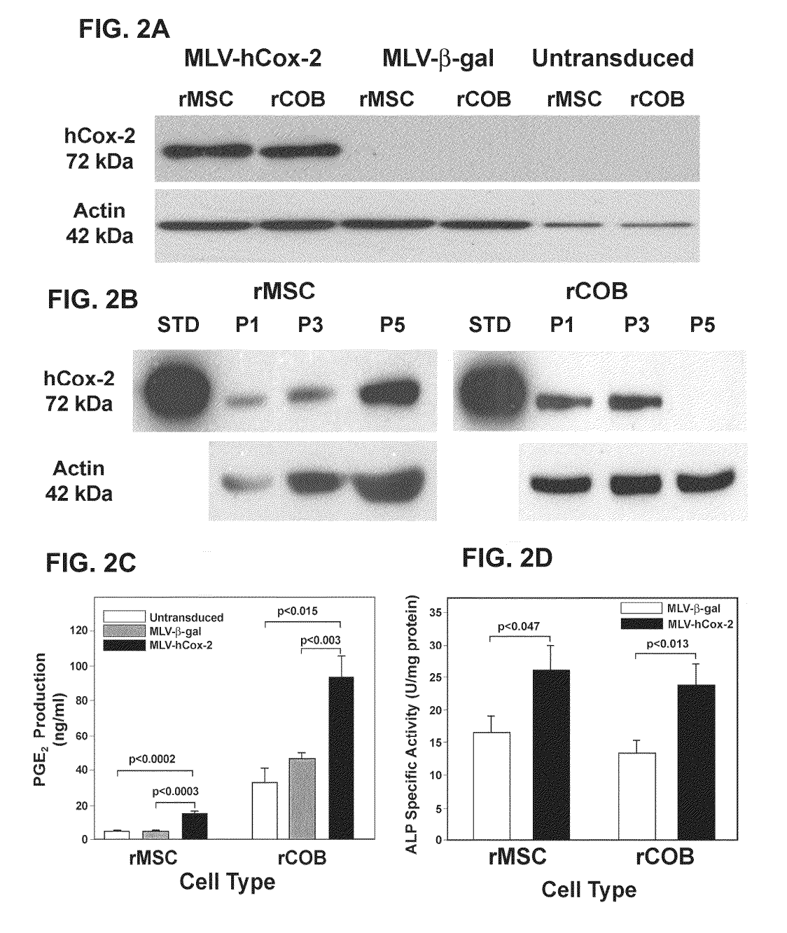

Cox-2 MLV and GFP-MLV virus was produced in 293T cells as previously described (Peng et al., Mol. Ther. 4:95-104, 2001). HT1080 cells, normally used to titer the retrovirus, rat calvarial osteoblasts and rat marrow stromal cells were transduced with the MLV Cox-2 retroviral vector (such as pCLSA-hCox-2, see FIG. 1 and FIGS. 12A-12D) and the MLV-β-galactosidase control vector. Transfection efficiencies ranged from 70% to 90% by FACS analysis for green fluorescent protein (GFP) expression in cells transduced in parallel with MLV-GFP (data not shown). Western immunoblotting assays indicated that the 72 kDa mature human Cox-2 protein was produced by the MLV-hCox-2-transduced rat marrow stromal cells and rat calvarial osteoblasts (˜200 to 800 ng Cox-2 protein/10⁶ cells), while the MLV-β-gal-transduced or untransduced rat marrow stromal cells and rat calvarial osteoblasts did not produce detectable amounts of the Cox-2 protein (FIG. 2A).

The duration of Cox-2 production varied between the cell types. Cox-2 expression in transduced rat calvarial osteoblasts was negligible after culturing cells for longer than 21 days (passage 3), while rat marrow stromal cells continued to express from 200-400 ng of human Cox 2 protein per 10⁶ cells for 35 days (passage 5) or more (FIG. 2B). β-gal expression continued throughout the 35 day culture period in both cell types and up to passage 10 (70 days) in each test cell type. These results suggest that the rat marrow stromal cells are a good ex vivo cell target for gene delivery because this cell target is capable of sustained expression of Cox-2 after viral transduction.

### Example 3

### Prostaglandin E2 Production in Cells Transduced With MLV-Cox-2 and Effects on Osteoblast Marker Gene Expression In Vitro

Rat marrow stromal cells and rat calvarial osteoblasts produced significantly higher levels of PGE2 as compared to control cells, seven days after Cox-2 transgene transduction (FIG. 2C). PGE2 production in Cox-2 transgene transduced rat marrow stromal cells was reduced at later times tested up to passage three (21 days) but was still significantly above levels in control cultures. PGE2 production in rat calvarial osteoblasts diminished after 21 days, which is consistent with the reduction in Cox-2 transgene expression at passage 5 and thereafter (FIG. 2A). In both the rat marrow stromal cell and rat calvarial osteoblast cell extracts from early passage cells, the level of alkaline phosphatase activity (a marker of osteoblastic differentiation) was dramatically stimulated by Cox 2 gene transduction (FIG. 2D). PGE2 is known to stimulate ALP activity in rat osteoblasts and marrow stromal cells (Shamir et al., Bone 34:157-162, 2004; Kaneki et al., J. Cell. Biochem. 73:36-48, 1999). Thus, osteoblasts and bone marrow stromal cells transduced with the retroviral vector construct produce biologically significant amounts of PGE2.

### Example 4

### Retroviral-Based Human Cox-2 Gene Therapy on Fracture Healing in the Rat Femur Fracture Model

Twelve-week old male Fischer 344 rats underwent femoral fracture, as described in Materials and Methods, and were sacrificed at different times during healing. X-ray analysis of fracture healing was evaluated at 7, 10, 17 and 21 days post-fracture. When compared with MLV-hCox-2-injected fracture tissues at 7 days and 10 days post-fracture, the hCox-2-treated fractures at 21 days post-fracture exhibited mineralized tissue that spanned the fracture gap and was not observed in the β-galactosidase-treated control fractures.

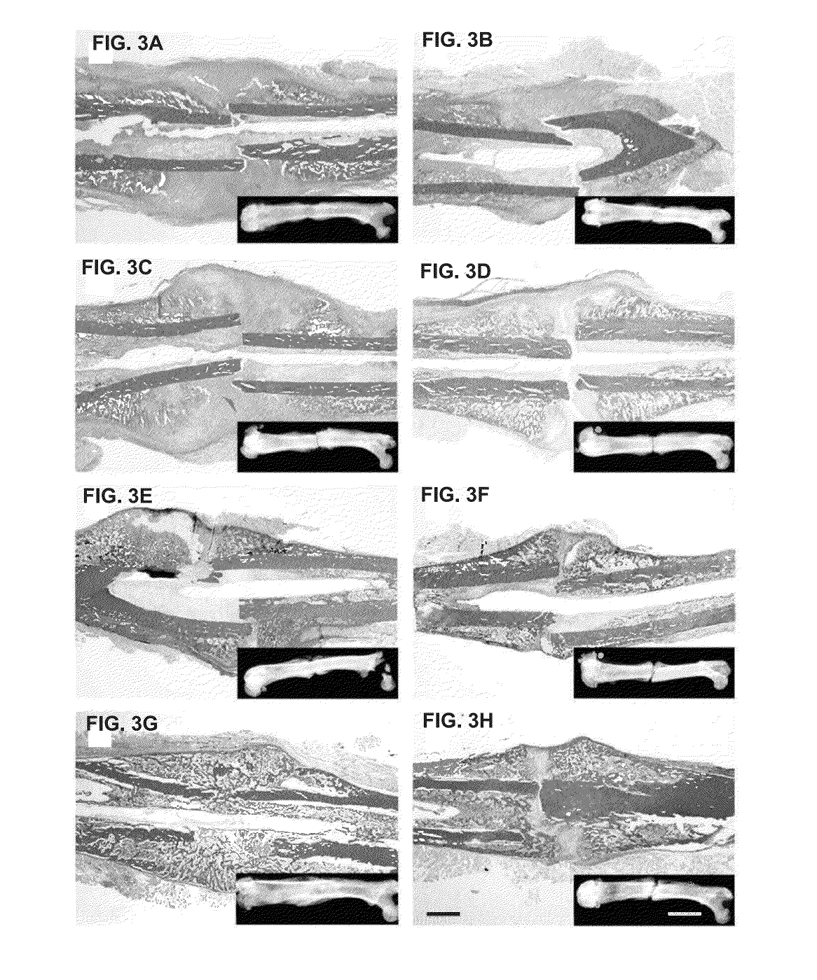

Radiographic evidence suggested that fracture healing is improved in MLV-Cox-2 injected rat femurs at 21 days. Examination of the fracture histology by Van Giesen staining confirmed that union was clearly complete at 21 days after fracture in the Cox-2-treated fractures but not in the control group. Additionally, bridging of the fracture was symmetric, occurring around the entire circumference of the fracture, even though the injection of the viral vector was from the lateral aspect (FIGS. 3A-3F). The bones from the animals receiving the MLV-hCox-2 exhibited either very little or no cartilage at the fracture site at 21 days, whereas the MLV-β-galactosidase-injected control animals had cartilage that persisted in the fracture gap. To confirm this observation, additional MLV-hCox-2-treated fractures were compared with control fractures. In total, 7 of 8 MLV-hCox-2-treated fractures showed evidence of bridging or very near bridging of the fracture at 21 days, while only 1 of 6 control fractures had bridged the fracture gap at this time. There were no differences in the fracture histology between test and control group healing at 7 and 10 days post-fracture. Immunostaining at 7, 10, 17 and 21 days revealed more Cox-2 immunoreactivity in the fibrous and cartilage tissues of the animals treated with the MLV-hCox-2 gene therapy compared to MLV-β-galactosidase. In contrast with the largely symmetric bony fracture callus, the immunoreactivity was noticeably asymmetric, occurring on the lateral aspect of the fracture callus. Without being bound by theory, this observation suggests that the symmetry of healing is imparted by prostaglandin migration from the cells transfected with the Cox-2 transgene at the injection site.

To better characterize the time course of the MLV-hCox-mediated fracture healing, healing in four additional MLV-hCox-2 fracture injections was compared with four additional MLV-β-galactosidase injections at 17 days post-fracture. At this time, it appeared that the therapy was beginning to show fracture healing enhancing effect as compared with 10 days. Mineralized tissue is observed in the fracture X-ray and bone formation suggested by Van Giesen staining. Additional analysis revealed that the fracture gap of the hCox-2-treated animals contained cartilage, and structures suggestive of sinusoids, strongly suggesting that this time (10-17 days) was a critical juncture for remodeling and angiogenesis. These tissues developed asymmetrically on the lateral aspect of the fracture, suggesting a concentration gradient of Cox-2-derived prostaglandin therapy emanated from the injection site early and produced the symmetric bony union at 21 days post-fracture, when its concentration had reached sufficient levels, apparently beginning at approximately 17 days. At this time point, Cox-2 gene therapy was beginning to show an effect on fracture healing when compared to 10 days of healing; mineralized tissue and bone formation were observed (indicated by van Giesen staining and the fracture X-ray, respectively). Between 17 and 21 days post-fracture the replacement of cartilage by bony tissue was symmetric. Quantification of fracture callus cartilage by measuring the Safranin Orange-stained area as the percentage of the total callus area demonstrated that the cartilage content was significantly different between Cox-2 and control fractures at 21 days post-fracture.

### Example 5

### Cox-2 Gene Expression in Fractured Tissues From the MLV-hCox-2 and MLV-β-Galactosidase-Treated Animals

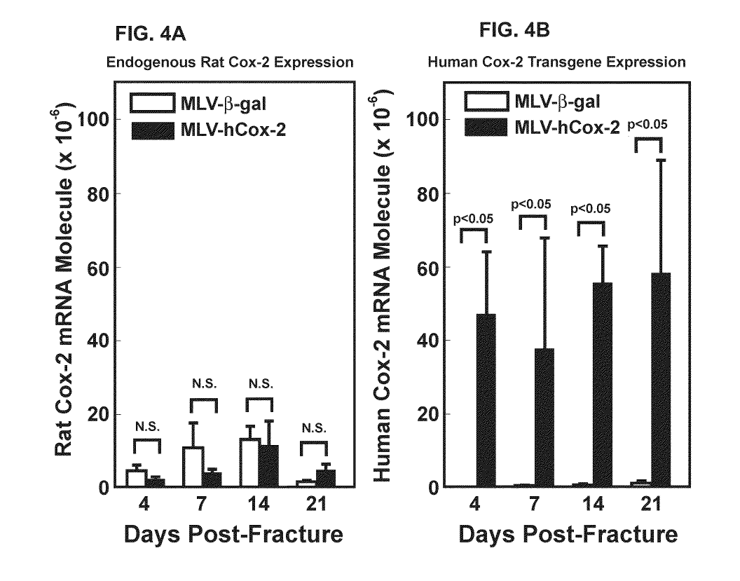

Tissue was collected from both groups of animals, four animals per group, at 4, 7, 14 and 21 days post fracture. These times were three days prior to those examined by histology, including the additional comparison at 17 days (above), when the results of therapeutic transgene expression would be expected to be observed as improved fracture healing. Approximately 25-30 μg of RNA was obtained from each fracture callus, and cDNA was prepared of at least 4 individual callus tissues injected with each vector at each post-fracture time. Endogenous rat Cox-2 (FIG. 4A) and human Cox-2 transgene (FIG. 4B) expression were determined by real-time RT-PCR and compared with expression of the housekeeping gene cyclophilin in MLV-hCox-2 and control MLV-β-galactosidase fracture injections. FIG. 4A illustrates that the expression of endogenous rat Cox-2 message demonstrated the expected biphasic response Importantly, it also demonstrated that endogenous rat Cox-2 gene expression levels were not significantly different in fracture tissues transfected with the MLV-hCox-2 vector and fracture tissues transfected with the β-galactosidase control gene, indicating that endogenous Cox-2 gene expression was not altered by Cox-2 transgene expression at any time. Accordingly, no feedback inhibition of endogenous Cox-2 expression had occurred. FIG. 4B shows that the expression of human Cox-2 message in callus tissues treated with the MLV-hCox-2 vector was markedly increased at 4 days post fracture. The expression was sustained throughout the 21 days of the examination period. In contrast, the MLV-β-galactosidase fractures showed no measurable human Cox-2 messages. In comparison with the amounts of endogenous rat Cox-2 message (FIG. 4A), the expression of human Cox-2 message in the fractures transfected with the MLV-hCox-2 vector was approximately 3-fold (p<0.05) that of endogenous rat Cox-2 message. The findings that the control fractures receiving the MLV-β-galactosidase control vector displayed no measurable human Cox-2 gene expression (FIG. 4B) confirms that the analysis accurately resolved human Cox-2 transgene expression from endogenous rat Cox-2 gene expression with no cross-reaction. Additional measurements also established that endogenous Cox-2 expression in the unfractured femur was negligible, establishing that transgene expression was due to vector transfection of the fracture tissues.

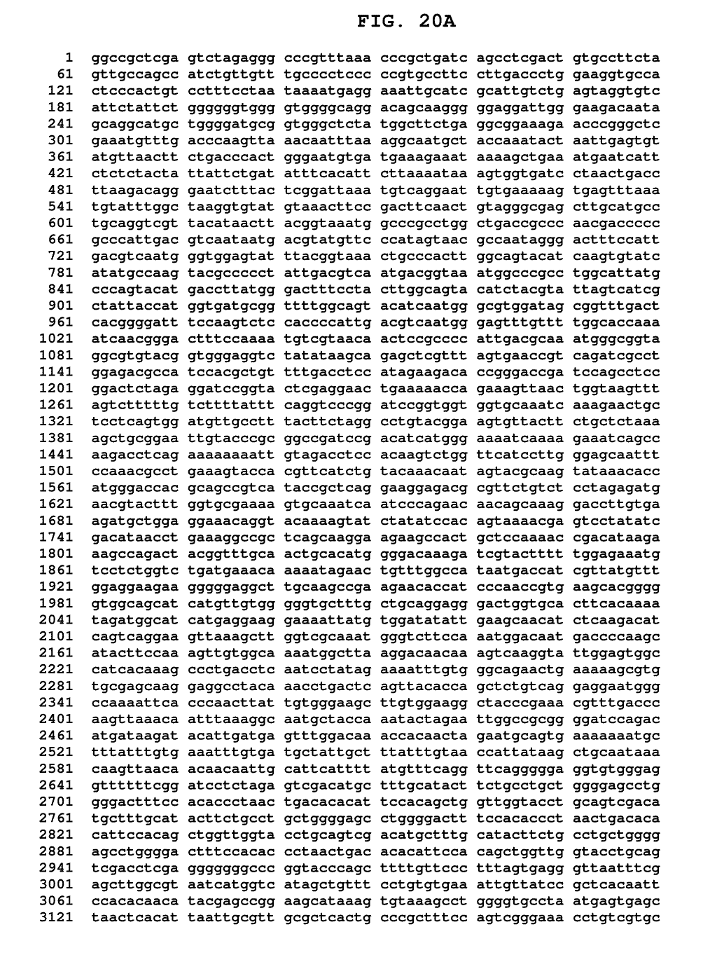

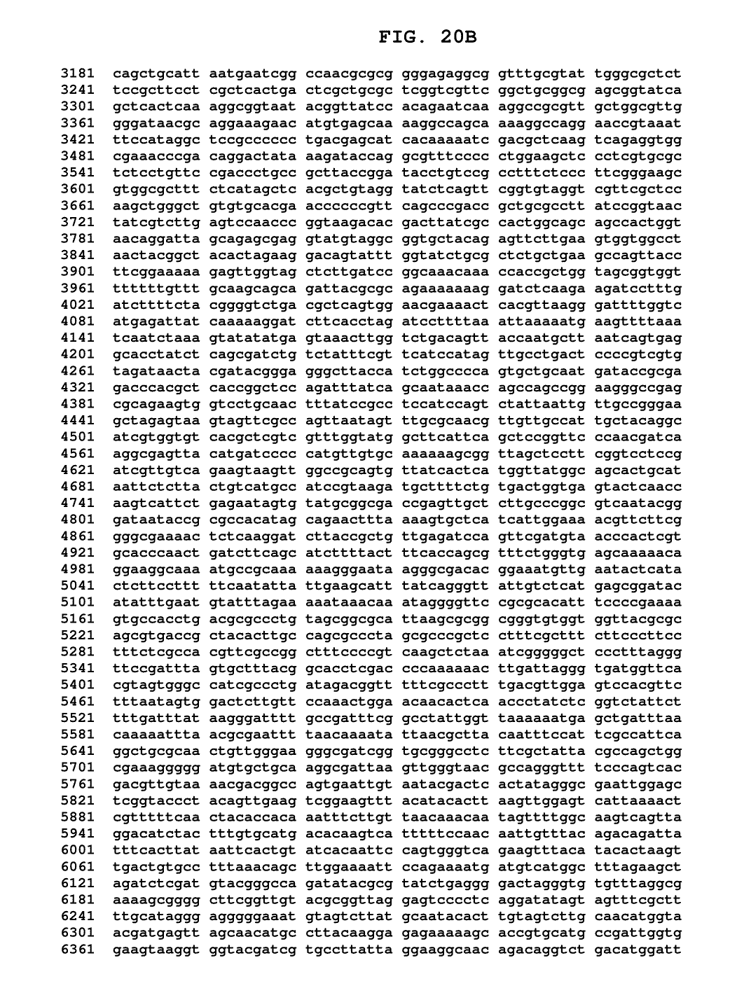

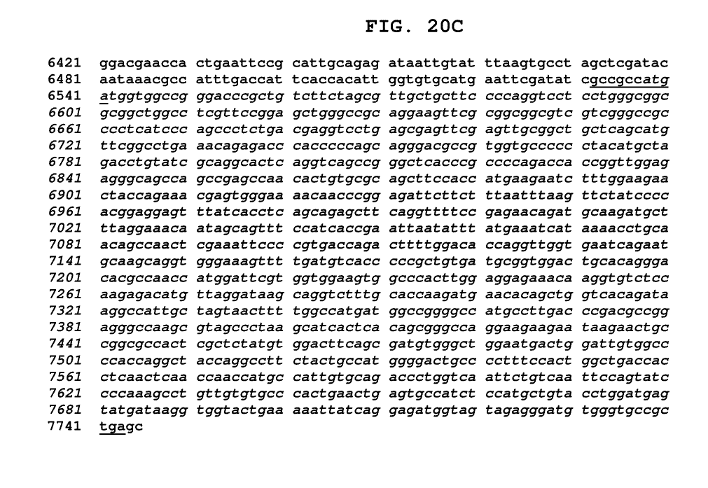

An MLV-based viral vector expressing a hybrid BMP-2/4 gene was developed (FIGS. 20A-20D), and its efficacy was previously tested in calvarial critical size defects ex vivo (Gysin et al., Gene Ther. 9:991-990, 2002) and fracture repair in vivo (Rundle et al., Bone 32:591-601, 2003). Healing of the critical size defect was observed within three weeks, when the defect had filled with high density bone. Fracture healing occurs when there is union of the fracture gap with bony callus; remodeling follows thereafter. BMP-2/4 gene therapy produced a large augmentation in endochondral bone formation at the fracture site that remodeled normally, yet did not produce bony union of the fracture gap any more rapidly than the controls. To accelerate bony union at the fracture gap and accelerate healing, one would require an earlier conversion of the soft callus to endochondral bone.

As in the BMP-2/4 gene therapy study, the delivery of gene therapy was successful by injecting a MLV-based vector expressing human Cox-2 into the fracture site in vivo. Because MLV vectors only transduce proliferating cells, this approach restricts gene expression to the cells that proliferate at the wound site after injury. In the case of fracture healing, periosteal cells that proliferate immediately at the injury and adjacent to the fracture site as early as 36 hours after fracture (Iwaki et al., J. Bone Miner. Res. 12:96-102, 1997) are susceptible to retroviral infection and present an excellent target for a retroviral vector to selectively express growth factor genes for fracture therapy.

As disclosed herein, an MLV vector expressing a modified human Cox-2 gene was constructed. In this transgene, the human Cox-2 gene sequence was modified to remove AT-rich sequences from the 3′ UTR region, which reduces RNA stability. Additionally, the native translation sequence was replaced with an optimized Kozak sequence to maximize protein production. These modifications greatly increased the level of Cox-2 gene expression, PGE2 production and the alkaline phosphatase activity in vitro. The in vitro results demonstrated that Cox-2 gene transduction increases PGE2 production and also stimulates the osteoblast phenotype in relevant cells, such as osteoblasts and mesenchymal stem cells.

In contrast to previous work with BMP-2/4 gene therapy, the Cox-2 gene therapy resulted in a significantly accelerated fracture healing in terms of bony union at the fracture site with no obvious adverse effects. One of the most remarkable aspects of this accelerated union is the fact that the animals that were selected for this model where young animals that have an inherent ability to rapidly heal fractures; control animals routinely healed their fractures within 35 days. It is well established that older animals experience delays in fracture healing. The appearance of sinusoid-like structures during the early phase of the therapeutic effect at 17 days suggest that Cox-2 was indeed mediating angiogenesis. These encouraging results indicate that fracture healing can be accelerated even in young individuals, who already have rapid fracture healing ability.

In the animals receiving the MLV-hCox-2, even though the virus was injected on the lateral side of the fractured femur, X-ray images and histological evaluations indicate accelerated healing around the entire circumference of the bone (FIGS. 3E, 3F). Without being bound by theory, this could be due, in part, to the fact that the MLV vector only transfects proliferating cells and proliferating cells would be found at the time of injection around the entire circumference of the fracture site, or that the virus was able to diffuse to the opposite side of the lesion and transduce cells around the entire circumference. However, the findings that the expression of both the β-galactosidase and BMP-2/4 transgenes in the fracture tissues in previous studies was asymmetric, implying asymmetric transfection that does not support these possibilities. In the current studies, immunohistochemical staining of Cox-2 localized Cox-2 expression in the lateral fracture and interstital cells of the adjacent muscle confirms an asymmetric transfection. Therefore, without being bound by theory, it is likely that symmetric bone healing was probably due to the fact that the prostaglandins produced by the cells transduced by the injection at the lateral aspect of the fracture site diffused into the surrounding tissues to promote more symmetric bone healing.

There was no evidence that tissues other than the fracture and supraperiosteal tissues were transduced with the MLV-β-galactosidase transgene. These tissues included the gonads, a tissue expected with mitotically active cells to be susceptible to MLV infection. These observations indicate that Cox-2 transgene expression was restricted to the injury.

Thus, enhanced Cox-2 gene expression caused an early maturation of the fracture callus bone at the fracture site and accelerated the bridging of the fracture gap with bony callus tissues. This effect was accomplished using a single percutaneous injection of the retroviral vector to transduce cells near the fracture site in vivo. However, unlike the BMP-2/4 transgene, the Cox-2 transgene delivered in this way did not produce the large amounts of endrochondral bone in the surrounding muscle. Because the animal model used in these studies constitutes a model in which fracture healing is not delayed due to age or other reasons, it seems likely that this therapy, while effective in younger subjects, could be even more effective in older subjects or in patients with diseases that impair fracture healing. The results demonstrate for the first time that local in vivo retroviral gene therapy with a single therapeutic gene actually accelerates fracture union.

### Example 6

### Intramedullary Injection of MLV-BMP2/4 Avoided Supra-Periosteal Bone Formation in the Rat Femur Fracture Model

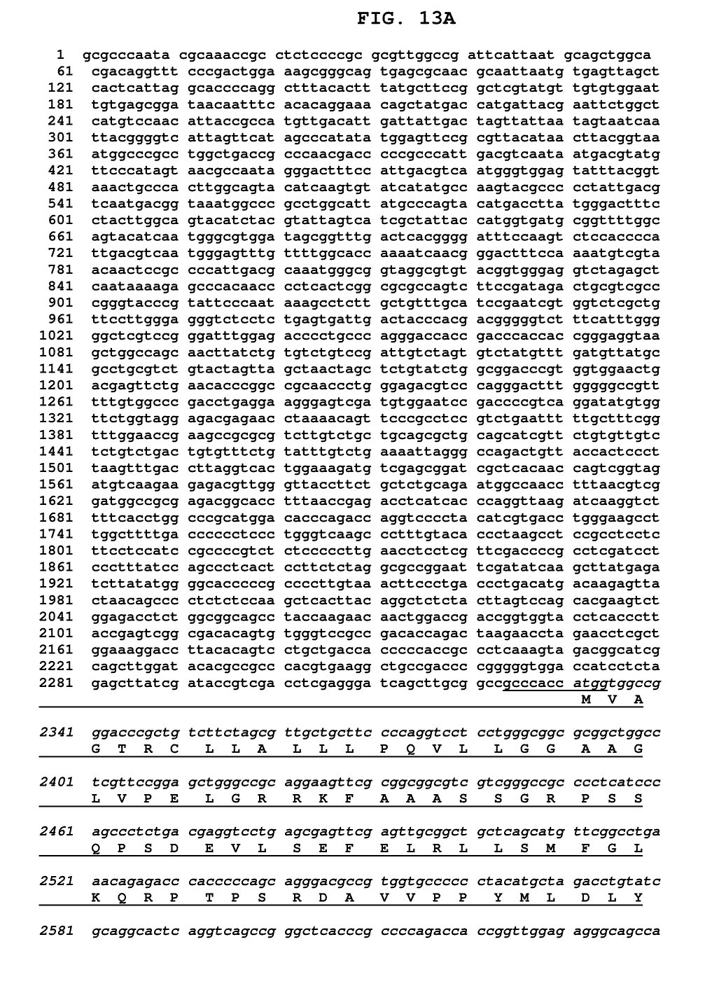

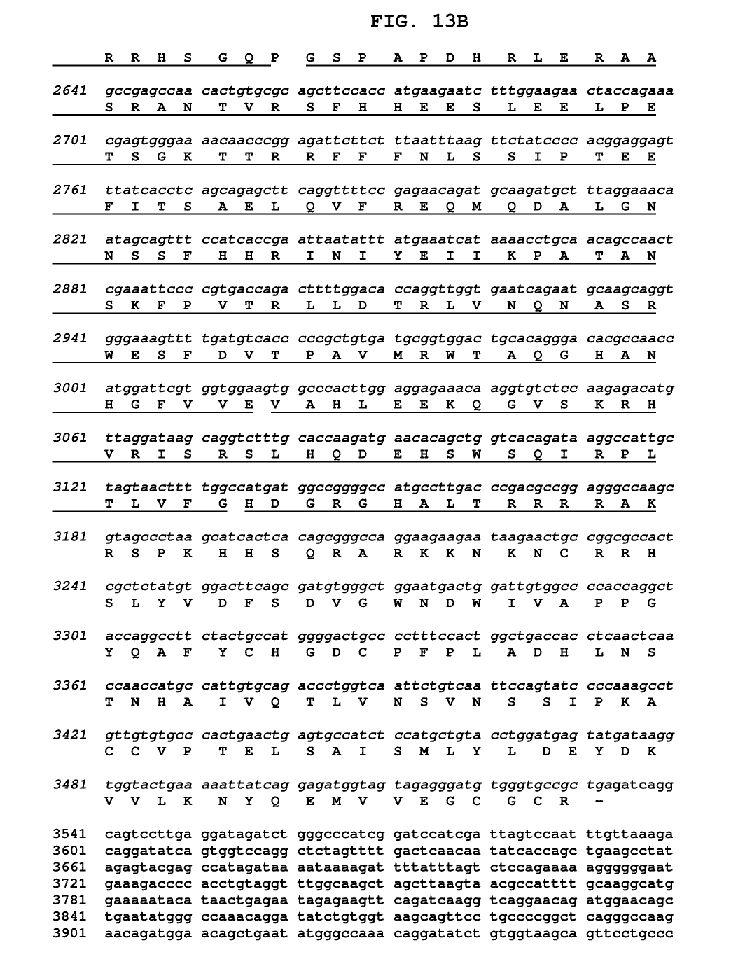

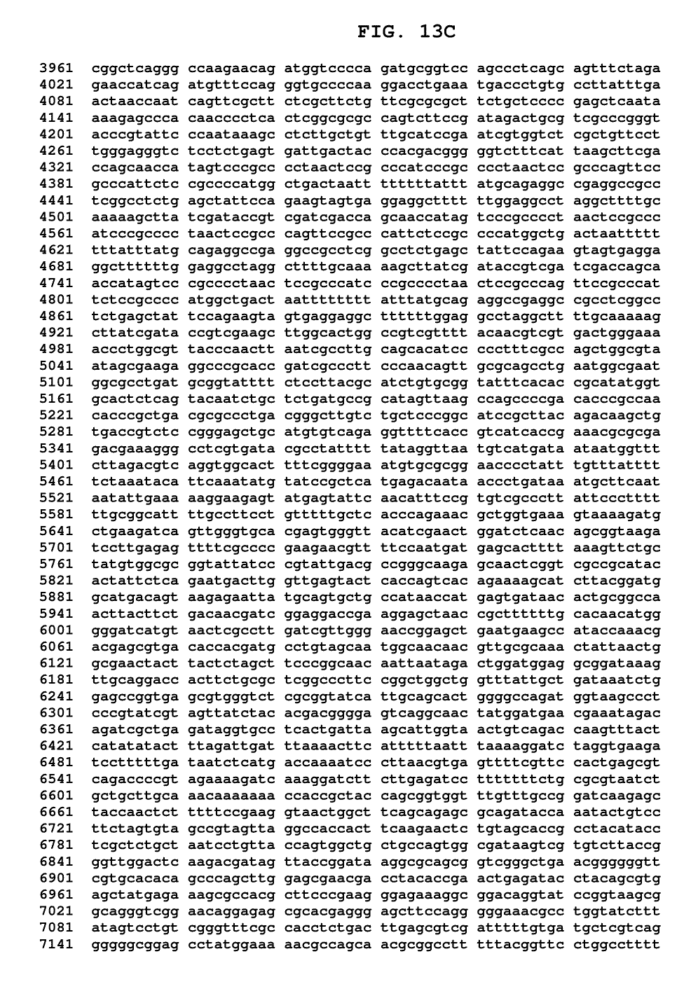

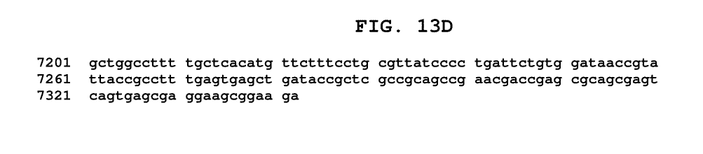

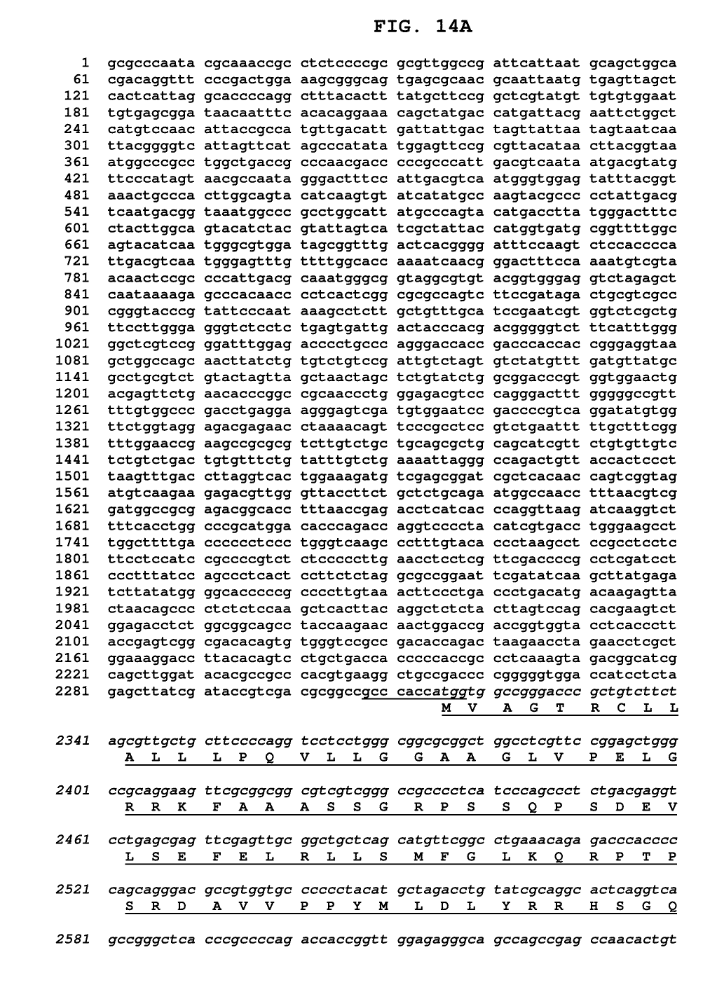

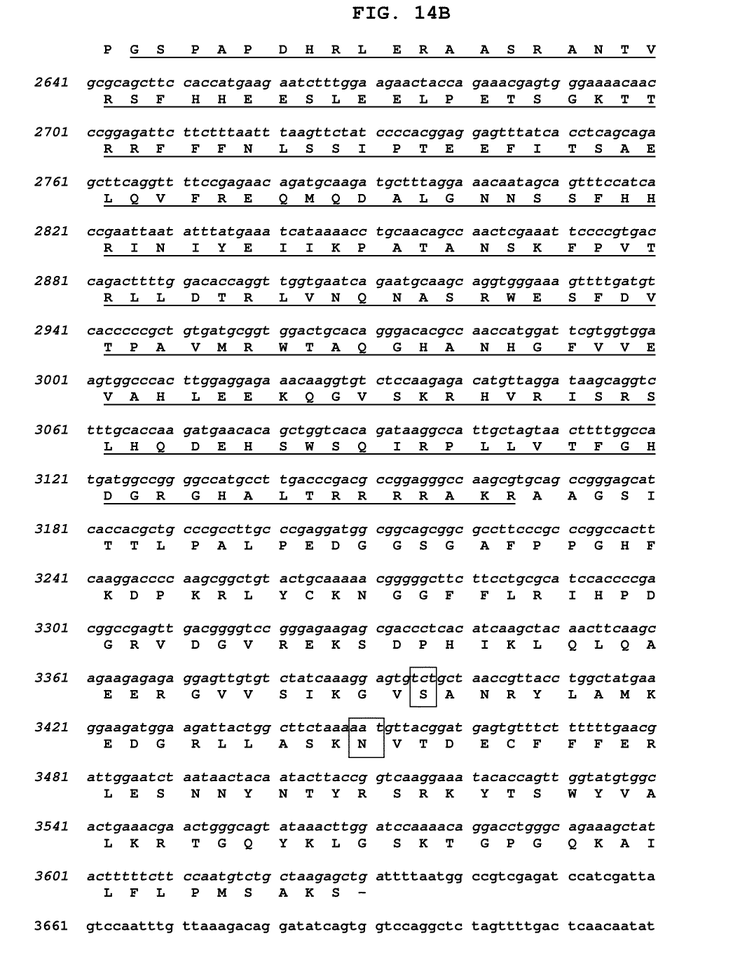

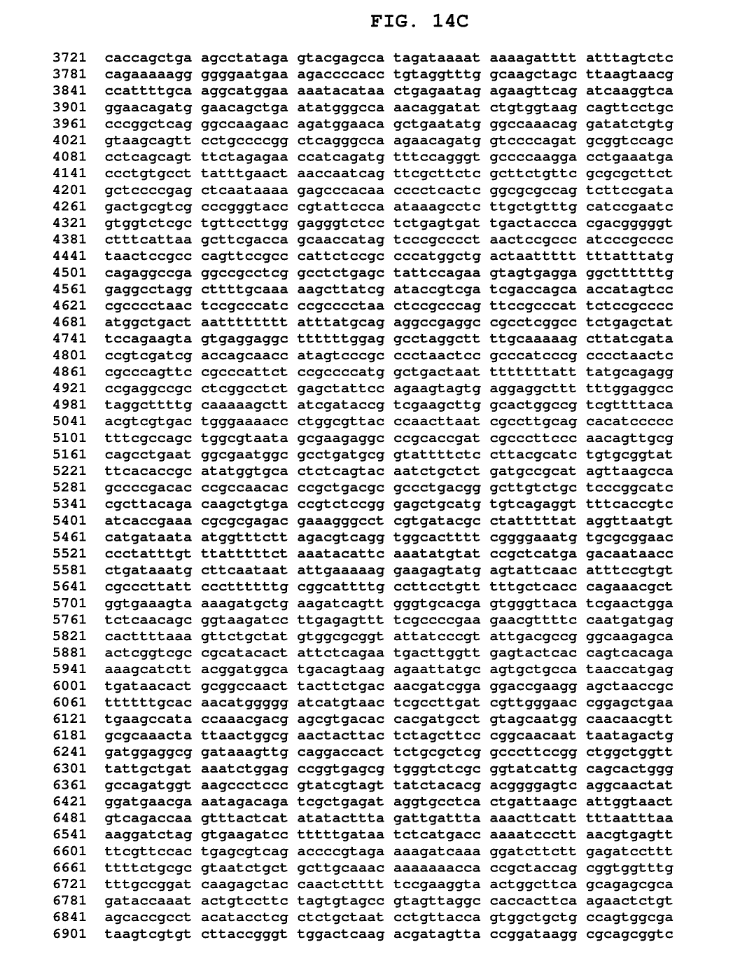

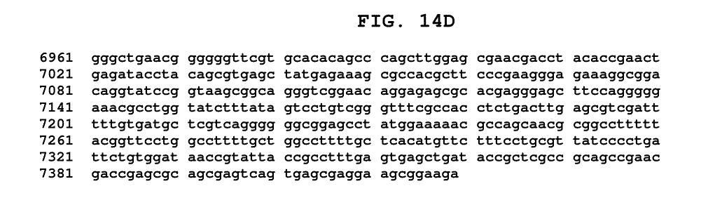

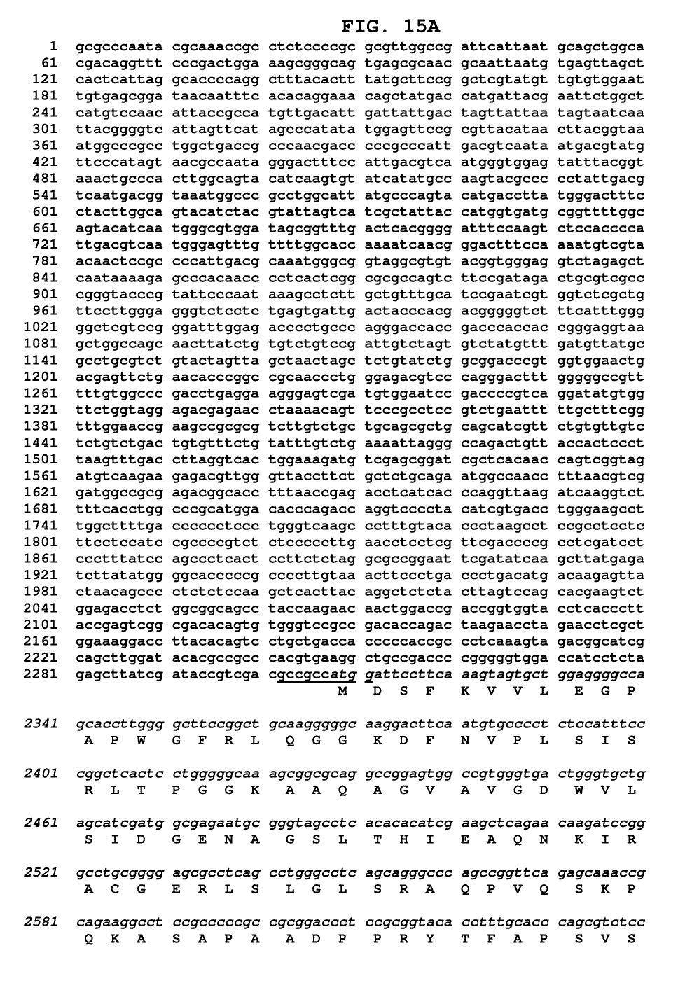

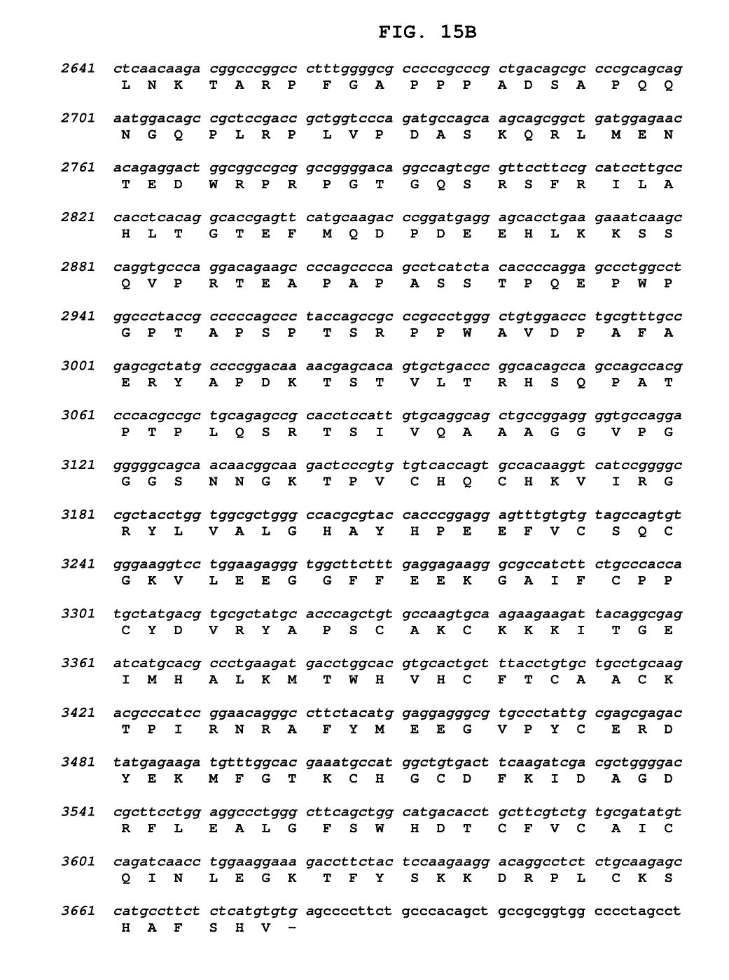

A catheter was surgically inerted into the medullary canal of the fractured femur (see above for methods). A single precutaneous injection of an approximately 1×10⁷ transforming units of the MLV-BMP2/4 virus (FIGS. 13A-13D) was injected into the intramedullary surface of the fracture site through the surgically placed intramedually catheter. For comparison, the same amount of MLV-BMP2/4 was injected from the exterior to the lateral aspect of the fracture in the control animal. Hard healing callus formation was monitored by X-ray analysis at 7, 14, 21, and 28 days post-injection. It appeared that the healing calluses of the intramedullary injected fractures were much more symmetric than those in the laterally injected fractures. As previously reported (Rundle et al., Bone 32:591-601, 2003), a single lateral injection of MLV-BMP2/4 virus at the fracture site resulted in asymmetric increase in the size of healing callus in the rat femur fracture model. This was due largely to the asymmetric transduction of tissues and the asymmetric release of BMP4 protein at the injection site. The lateral injection also caused the development of a large mass of mineralized tissue in the supraperiosteal tissues, augmenting bone around the fracture site failing to bridge the fracture gap. This is most probably because BMPs promoted trans-differentiation of muscle cells into cells of osteoblast-lineage and stimulated extraskeletal ectopic bone formation at the muscle tissues (for example, see Gonda et al., J. Bone Miner. Res. 15:1056-1065, 2000). Thus, this extraperiosteal bone formation was due to the injection of the virus into the muscle surrounding the fracture site. In contrast, there was a lack of evidence for supraperiosteal bone formation in the intramedullary injected fractures. In addition, expression from the subperiosteal bony tissues through the intramedullary injection formed mineralized tissue within the fracture gap most obvious at 14 day and appeared to produce better fracture healing. This delivery technique was therefore very effective for genes encoding secreted growth factors.

Histological analysis of the intramedullary injected callus revealed that the subperiosteal healing bony callus was close to bridging the fracture gap with augmented bony tissue at 28 days post fracture. The mineralized tissue that developed in response to gene therapy appeared to be a normal bony healing callus. The findings that intramedullary injection of MLV-BMP2/4 virus avoided supra-periosteal bone formation around the fracture healing suggest that Cox-2 gene can be used in conjunction with the BMP2/4 hybrid gene to promote the bridging fracture gap and as such, accelerate fracture repair, when the BMP2/4 hybrid gene is delivered by intramedullary injection.

### Example 7

### Retroviral-Based LMP-1 Gene Therapy on Fracture Healing in the Rat Femur Fracture Model

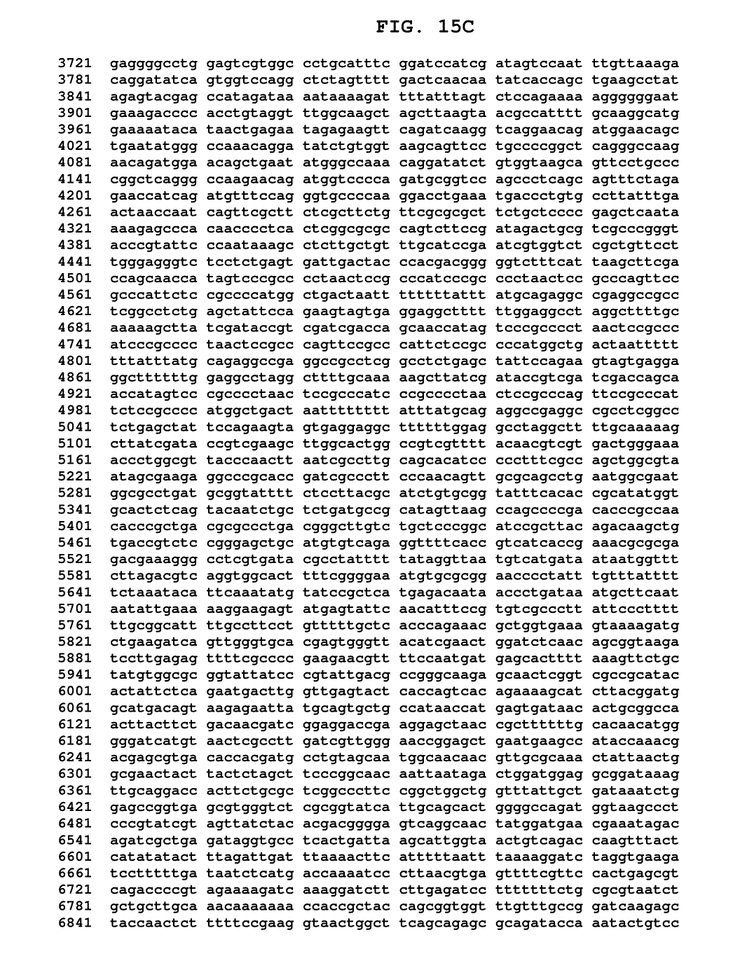

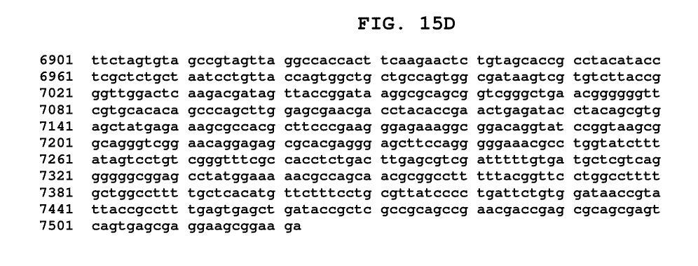

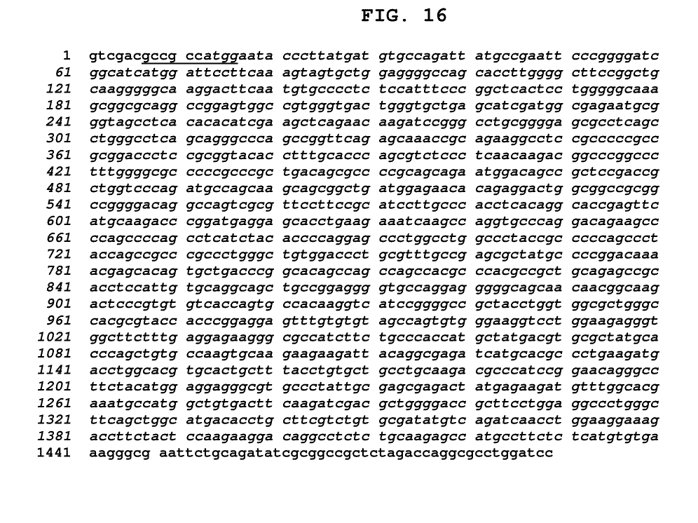

An MLV-based viral vector expressing LMP-1 gene (FIGS. 15A-15D, see also FIGS. 16-18) was developed for testing and used to introduce an LMP-1 in order to promote fracture repair. To assist the identification of LMP-1 protein expression, an MLV-based vector expressing an HA-tagged LMP-1, in which the HA tag sequence was added to the N-terminus of the LMP-1, was also produced. This allowed the use of the anti-HA antibody to detected the HA-tagged LMP-1 protein. The sequences of the MLV-LMP-1 vectors are shown in FIGS. 16-18.

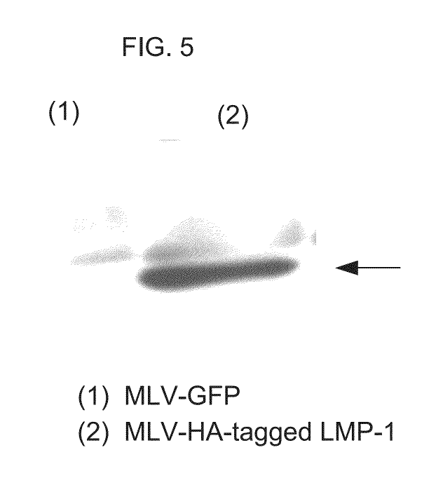

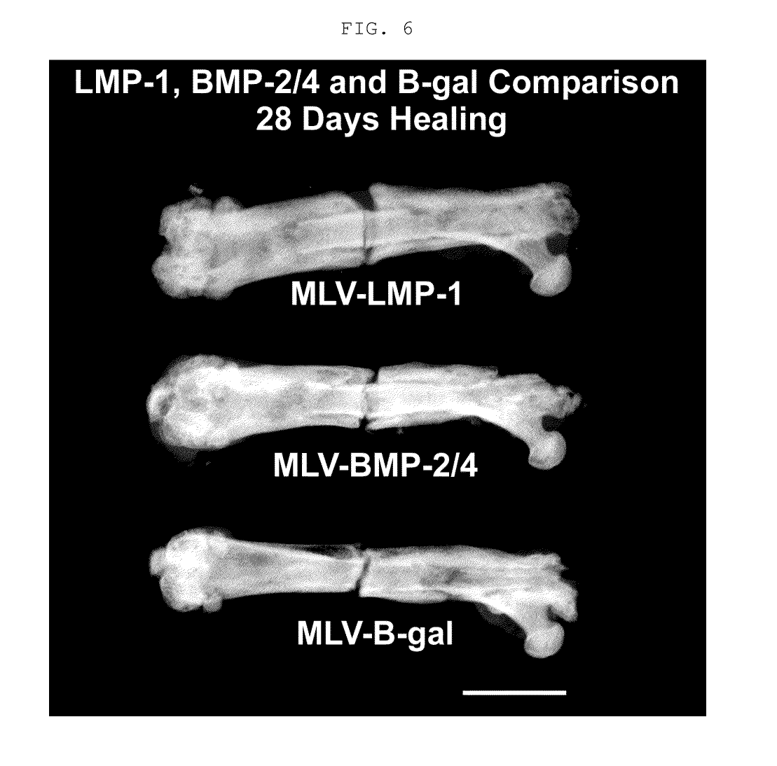

HT1080 cells were transduced with the MLV-HA-LMP-1, or MLV-GFP control virus. Forty-eight hours after the transduction, cells were lyzed and the expression of HA-LMP-1 protein was measured with the Western immunoblot assay using a mouse monoclonal anti-HA antibody. Cells transduced with the MLV-HA-LMP-1 virus, but not those with the MLV-GFP virus, produced an obvious 53 kDa HA-tagged LMP-1 protein (FIG. 5), indicating that MLV-based vectors effectively expressed the LMP-1 protein. Marrow stromal cells and calvarial osteoblasts transduced with the MLV-HA-LMP-1 produced the expected 52 kDa HA-LMP-1 protein recognized by anti-HA tag antibodies in Western immunoblots. Osteoblasts transduced with HA-tagged LMP-1 and cultured for 21 days, under conditions that promote mineralization, showed increased alizarin red staining and von Kossa staining, indicating increased mineral deposition in response to HA-LW-1. By contrast, cells transduced with control vector did not show any more mineralization than untransduced cells. These results suggested that the HA-LMP-1 was expressed in cells normally expected to mediate fracture repair, and that HA-LMP-1 was functional in increasing osteoblast differentiation.

Rat femur fractures were injected intramedullary with 1×10⁷ transforming units of MLV-LMP-1 virus through a surgically placed catheter a day after fracture. For comparison, two groups of rat femur fractures were injected intramedullary with the MLV-BMP2/4 virus and MLV-β-galactosidase control virus, respectively. The relative amount of hard callus formed in the MLV-LMP-1 injected fractures was assessed by X-ray on day 28 of healing and compared with that formed in the MLV-BMP2/4 injected fracture and that in the MLV-β-galactosidase injected fracture. The hard callus mineralization in the MLV-LMP-1 injected fractures was comparable to that of the MLV-BMP2/4 injected fractures, but was significantly greater than MLV-β-galatosidase injected (FIG. 6) or uninjected control fractures, suggesting that LMP-1 actions in fracture healing were similar to that of BMP2/4. Thus, LMP-1 gene therapy was as effective as BMP-2/4 gene therapy in fracture repair.

Histological analysis of the healing callus demonstrated that, in contrast to the MLV-β-galatosidase injected fractures, the MLV-LMP1-transfected fracture gap contains large amounts of cartilage, suggesting an increase in the endochrondral bone formation. These findings suggested an enhancement in fracture repair. Without being bound by theory, it is noted that LMP-1 is an intracellular protein that acts to enhance local expression of multiple BMPs to promote bone formation (see Pola et al., Gene Ther. 11:683-693, 2004) and functions as a co-activator to stimulate transcription from the type I procollagen promoter (Strong et al., Proceedings of the 87^{th} Annual Meeting of the Endocrine Society., P3691, 2005). Thus, the LMP-1 gene therapy could locally increase the concentrations of BMPs at the fracture site and could be used to avoid supraperiosteal bone formation. Thus, the LMP-1 gene could be used in conjunction with Cox-2 gene to accelerate fracture repair and to promote bridging of the fracture gap.

In an additional study, the effectiveness of LMP-1 gene therapy to promote bone formation and bony union in the rat femur fracture model. A murine leukemia virus (MLV)-based retroviral vector was used to target the expression of the human LMP-1 or control transgenes into cultured murine osteoblasts. As described above, LMP-1 was 5′-tagged with influenza hemaglutinin (HA-LMP-1) to facilitate its identification. Effects of increased HA-LMP-1 and control transgene expression on osteoblast mineralization in vitro were evaluated at day 21 by alizarin red and von Kossa staining.

Femur fractures were produced in 12-week-old male Fischer 344 rats by the three-point bending technique as described above. The retroviral vector was applied directly into the periosteum at the fracture site in a percutaneous injection at one day post-fracture; this approach targeted our retroviral vector to periosteal cells stimulated to proliferate in response to injury. Healing in HA-LMP-1 treated and β-galactosidase control rats was evaluated by X-ray analysis at 7, 10, 14 and 21 days, and by pQCT and histology at 21 days after fracture. Statistical analysis was performed by t-test Immunohistochemistry with anti-HA and anti-BMP-4 antibodies was used to identify the cells that expressed HA-LMP-1 and BMP-4 protein, respectively.

Radiographic evidence suggested that mineralized tissue was augmented at 21 days in fractures of each animal injected with MLV-HA-LMP-1, as compared to β-galactosidase control animals. No heterotopic bone formation was observed. Examination of the LMP-1-treated fracture histology at 21 days by Safranin Orange staining also suggested that the increased mineralized tissue was increased bone formation that coincided with reduced cartilage, fibrous tissue and improved union at the fracture gap at 21 days. This effect occurred well before 35 days normally required for bony union (healing) in this fracture model. Previous studies also established that the HA tag did not affect fracture repair. None of the control fractures displayed augmented bone formation at 21 days.

pQCT analysis at the fracture site at 21 days post-fracture revealed that HA-LMP-1 therapy significantly increased the bone mineral content of the mineralized callus to 4.57±0.76 from 2.39±0.87 in the controls (p<0.04), and the area of the mineralized callus to 12.11±2.02 from 7.64±1.55 in the controls (p<0.05). The area of the soft callus tissues was not significantly different at 21 days of healing. None of these callus parameters were significantly different in response to HA-LMP-1 therapy earlier than 21 days post-fracture.

Immunostaining of 21-day fracture tissues revealed intensely staining HA-LMP-1 immunoreactivity at the fracture site in osteoblast lineage cells, especially cells embedded in osteoid and cartilage cells of animals treated with MLV-HA-LMP-1. Cells at the osteoid surface stained intensely with anti-BMP-4 antibody in both MLV-HA-LMP-1 and control fractures. However, BMP-4 and HA-LMP-1 expression did not co-localize in the fracture tissues.

Thus, local expression of LMP-1 from a retroviral vector increased bone formation and enhances bony union in a normally healing fracture model. When applied to an established in vivo model of fracture repair by the same injection technique, LMP-1 1) enhanced the union of bony callus tissues over the fracture, and 2) promoted this healing without the production of heterotopic bone. Furthermore, while immunohistochemistry demonstrated that LMP-1 expression was consistent with enhanced differentiation of the osteoblast lineage, the lack of colocalized BMP-4 expression and heterotopic bone production strongly suggested that LMP-1 therapy was not mediated directly through BMP-4 production These studies demonstrate that MLV-LMP-1 gene therapy is effective for the treatment of bone fractures.

### Example 8

### Retroviral-Based FGF-2 Gene Therapy on Fracture Healing in the Rat Femur Fracture Model

Although administration of human recombinant FGF-2 protein promoted fracture healing in the monkey (see Kawaguchi et al., J. Clin. Endocrinol. Metab. 86:875-880, 2001), FGF-2 gene therapy has not previously yielded reportable successes. Unlike most extracellular growth factors, FGF-2 lacks a classical secretion signal sequence and its extracellular secretion is mediated by an energy-dependent, non-ER/Golgi pathway (see Mignatti et al., J. Cell. Physiol. 151:81-93, 1992). This export mechanism is highly inefficient and, as a result, the amount of FGF-2 released into the extracellular fluid by this mechanism is extremely low and inconsistent (see Florkiewicz et al., J. Cell. Physiol. 162:388-399, 1995). The FGF-2 protein can also exist in various molecular forms through intramolecular and/or intermolecular disulfide formation, which causes conformational changes, significant loss of biological activities, and protein instability (see Iwane et al., Biochem. Biophys. Res. Commun. 146:470-477, 1987). The inefficient FGF-2 secretion, along with the formation of FGF-2 disulfide complexes, result in inconsistent biological effects of FGF-2 therapy through gene transfer approaches.

To overcome these problems, a MLV-based chimeric FGF-2 vector expressing a modified FGF-2 gene (i.e., pCLSA-BMPFGFC2SC3N, see FIGS. 21A-21D) was produced. The sequence of this vector is shown in the Sequence Listing section. The modified FGF-2 chimeric gene contained the BMP2/4 classical secretion signal sequence. In addition, two essential cysteines (cys-70 and cys-88), which are essential for intra and/or intercellular disulfide formation, were also mutated to serine and asparagine. A MLV-FGF expressing the wild type FGF-2 was also generated for comparison.

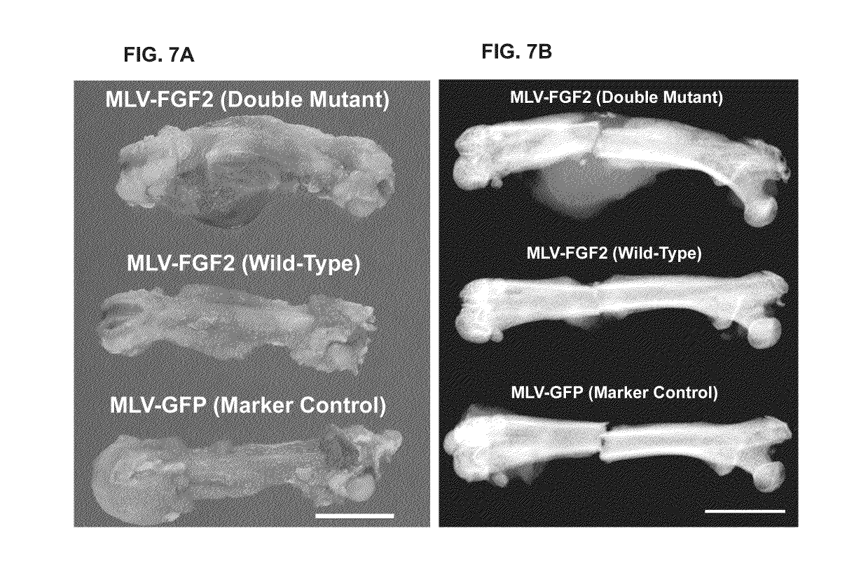

Three groups of 12-week-old male Fischer 344 rats with fractures were used in these studies. A single dose of 1×10⁷ transforming unit of each of the pCLSA-BMPFGFC2SC3N double mutation vector, the pCLA-BMPFGF vector, and the pCLSA-GFP control vector was injected into the fracture intramedullary via a surgically placed catheter. After 11 days of healing, the animals were sacrificed and the healing fractured femurs were isolated. Gross anatomy evaluation of the healing fracture callus at 11 days of healing showed that the FGF-2 with double mutations (FIG. 7A, top) produced a massive fracture callus, while the wild type FGF-2 (FIG. 7A, middle) yielded a much smaller fracture callus compared with the FGF-2 with double mutations. However, this callus formation it was still significantly larger than that of the control fracture callus of the GFP control group (FIG. 7A, bottom). X-ray analysis of the mineralized tissues within the hard fracture calluses revealed that the FGF-2 with double mutations (FIG. 7B, top) showed evidence of increased mineralized tissues, which were barely visible in the hard callus of the wild type FGF-2 fractures (FIG. 7B, middle). The MLV-GFP-injected fracture showed no detectable hard callus (FIG. 7B, bottom). Therefore, FGF-2 promoted fracture healing. However, the C2SC3N mutant was potent than wild type FGF-2 in promoting fracture healing, in part due to its high secretion rate and stability.

Histological analysis of the healing calluses of the pCLSA-BMPFGFC2SC3N treated animals revealed a robust infiltration of osteoblasts in the hypertrophic cartilage, indicating endochondral bone formation. There was evidence of capillaries containing red blood cells within the healing fracture, which was consistent with an angiogenic action of FGF-2. These findings together strongly suggest enhanced vascularity assisted osteoblast infiltration into the hypertropic cartilage, which resulted in an enhancement of endochondral bone formation.

Histological analysis of the healing calluses of FGF-2 treated animals revealed that FGF-2 gene therapy, like BMP2/4 gene therapy, was unable to promote the bridging of fracture gaps. Cox-2 also promoted untion of fracture gaps.

Based on the findings presented herein, in vitro screenings for agents that promote bone formation, bone resorption, and angiogenesis, can be used to screen for effective agents that promote fracture repair, possibly in conjunction with the in vivo fracture model.

### Example 9

### Lentiviral-Based Gene Therapy of Fracture Repair

Third generation lentiviral vectors expressing either the β-galactosidase control gene or the BMP2/4 gene were produced as described above. To identify the localization of lentiviral vector-transduced cells within the fracture site, the femur fractures of Fischer 344 rat was injected intramedullary with an approximately 1×10⁷ transforming units of the lentiviral-based vector expressing the β-galactosidase reporter gene (pHIV-β-gal) through a surgically placed catheter one day after the fracture. For comparison, another group of femur fractures received intramedullary injection of 1×10⁷ transforming units of the MLV-β-galactosidase vector. The femurs were harvested at one week post-fracture.

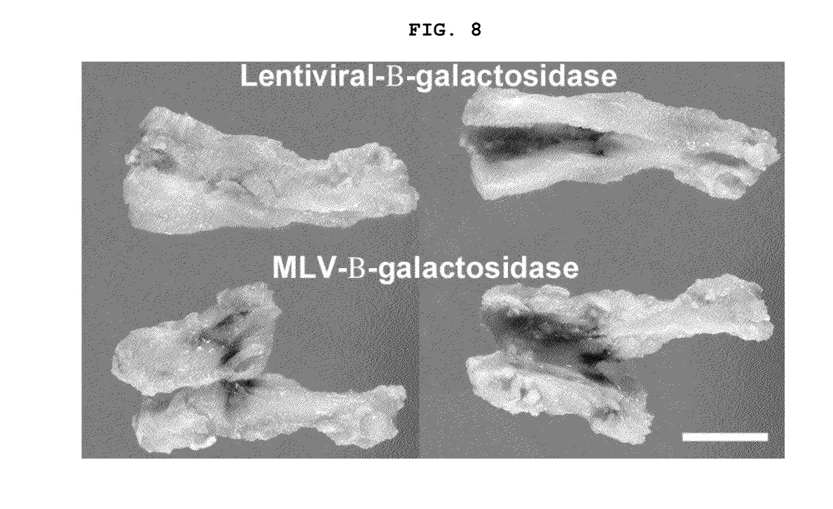

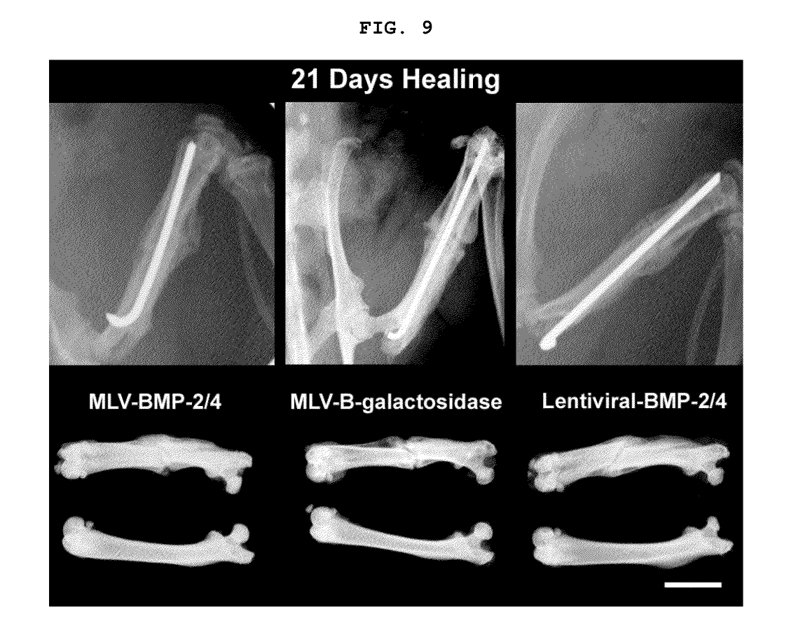

The staining of β-galactosidase activity revealed that strong β-galactosidase expression was found in both groups (FIG. 8), indicating effective transduction of cells at the fracture sites by either viral vector. In both cases, the localization of the transduced cells was mostly around the fracture site.

To evaluate the effectiveness of lentiviral-based vectors for promoting bone formation in the fracture site, three groups of rat femur fractures received an approximately 1×10⁷ transforming units each of pHIV-BMP2/4 (lentiviral-based BMP2/4 vector), MLV-BMP2/4, or MLV-β-galactosidase. The vectors were injected into the marrow space of the fractures via the surgically placed catheters. Bone formation and fracture healing were monitored with X-rays at 21 days of healing. There was evidence that fractures receiving either the pHIV-BMP2/4 or the MLV-BMP2/4 injection promoted fracture repair (FIG. 8, left and right panels, respectively), whereas the fracture receiving the control MLV-β-galactosidase vector showed no fracture gap closing (FIG. 8, center panel). These findings indicate that the lentiviral based vectors can be used to promote fracture repair. Advantages of lentiviral vectors include use of tissue-specific nonviral promoters, which can increase cell type specificity of the gene therapy, as well as their ability to transfect non-dividing cells, which allows delivery outside of the proliferative injury phase.

### Example 10

### A Sleeping Beauty Tc1-Like Transposon-Based Nonviral Vector With One or More Copies of DNA Nuclear Targeting Sequences (DTSs)

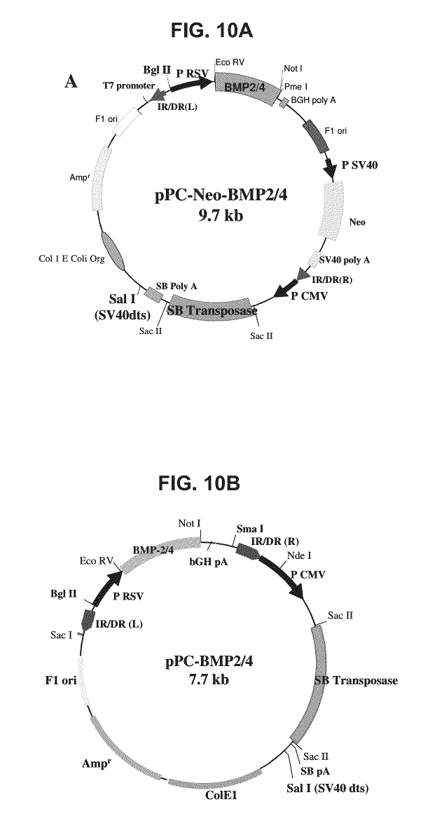

Non-viral vectors have different safety profiles than viral vectors (see Klamut et al., Crit Rev Eukaryotic Gene Expression 14:89-136, 2004). Sleeping Beauty Tc1-transposon-based non-viral vector systems have the advantage that this system permitted incorporation of the non-viral plasmid transgene expression cassette into the genome of the host cells at relatively specific sites (AT-dinucleotide sites, see Plasterk, Curr. Top. Microbiol. Immunol. 204:125-143, 1996). The original Sleeping Beauty vector system was a multiple plasmid system. The single plasmid-based Sleeping Beauty vectors, termed “Prince Charming” were subsequently developed (Harris et al., Anal. Biochem. 310:15-26, 2002, herein incorporated by reference). Like most other plasmid nonviral vectors, the transfection efficiency of the original Prince Charming vector was limited by the nuclear transport of the vector. It is disclosed herein that the incorporation of one or more copies of the SV40 DTS (also indicated “SV40dts”) markedly enhanced transfection efficiency and transgene expression. FIG. 10 depicts the schematic structure of the Tc1-like transposon-based Prince Charming nonviral vector expressing the BMP2/4 hybrid gene that contained the SV40 DTS.

In this construct, up to three copies of the 72 by SV40dts were inserted in tandem as Sall fragments generated by PCR in the forward orientation in the Sall site of the pPC-BMP2/4 vector. These SV40dts's were outside the transposase and outside the transposon in the pPC vector and therefore were not incorporated into the host genome. The BMP2/4 coding sequence was inserted in the EcoRV/NotI site flanked by the transposons and will be incorporated into the host genome after transfection.

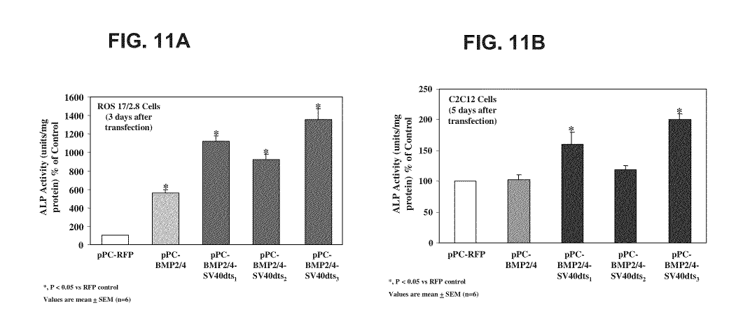

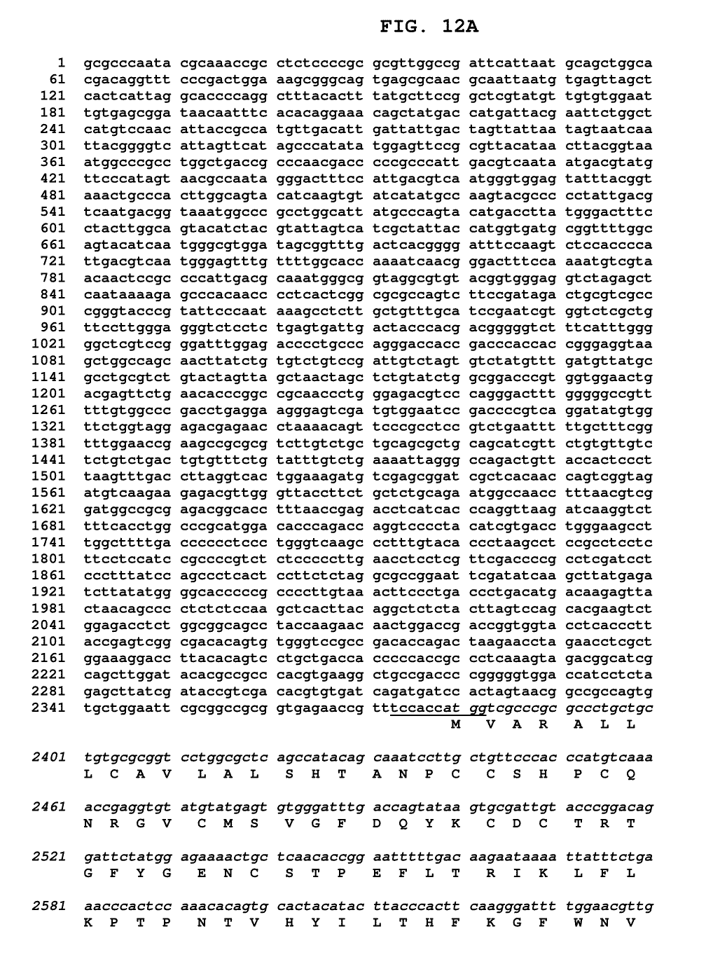

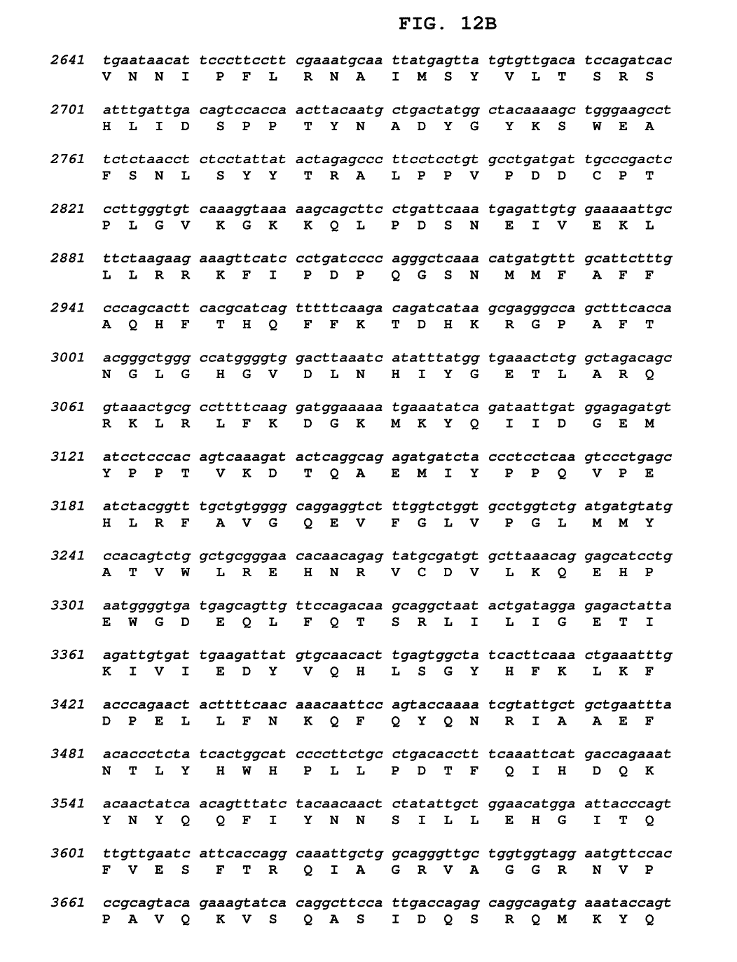

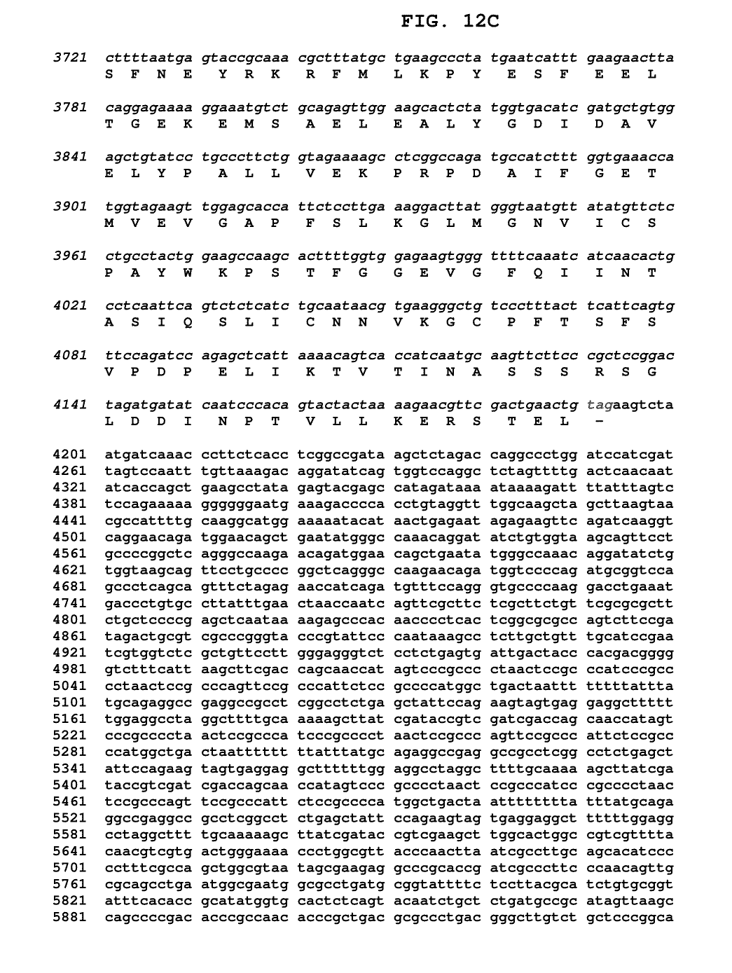

To test the transfection efficacy of the pPC-dts-BMP2/4 nonviral vector in osteoblasts and myogenic cells, ROS 17/2.8 osteoblastic cells (FIG. 11A) and C2C12 myogenic cells (FIG. 11B) were transfected with the pPC-dts-BMP2/4 using effectene. For comparison, each cell type was also transfected with the pPC-BMP2/4 vector without the SV40 DTS. The transfection efficiency was assessed by the increase in alkaline phosphatase activity, which is a biological functional assay for BMP4 expression.

Although the pPC-BMP2/4 vector without the SV40dts significantly increased ALP activity in both cell types (FIG. 11), the pPC-BMP2/4 vector with the SV40 DTS increased BMP2/4 expression to increase ALP in these two cell types up to 14-fold in ROS 17/2.8 cells (FIG. 11A) and up to 2-fold in C2C12 cells (FIG. 11B). Sufficient amounts of BMP4 were produced to transdifferentiate the C2C12 cells. One or three copies of the SV40dts were much more effective than two copies of SV40dts in both cell types. These findings indicate that the incorporation of DTS, such as that of SV40, increases the transfection efficiency of Tc1 transposon-based nonviral vectors. These nonviral vectors can be used in place of viral vectors in gene therapy of fracture repair.

The results presented herein show that that an MLV-based vector including hCox2 can be used to promote fracture healing and/or spinal fusion. The results presented herein show that intramedullary injection of the Tc1-like transposone based pPC-hCox2 expression plasmid with the SV40dts into the fracture site via catheter promotes fracture union and enhance fracture repair. Many of the 23 members of the fibroblast growth factor multigene family (FGF-1 to FGF-23) have been shown to increase bone formation, increase bone resorption, and increase angiogenesis. Thus, intramedullary injection of MLV-based vector expressing any of the wild type or functional mutants of one of these 23 members of the fibroblast growth factor family will promote fracture healing. These vectors can be used alone or in combination with a vector encoding Cox-2. Direct intramedullary injection of a combination of the MLV-FGF-2, MLV-LMP1, or MLV-BMP2/4, virus can enhance the ability of MLV-hCox2 to promote fracture repair. In addition, direct intramedullary injection of a combination of MLV-hCox2 and MLV-BMP2/4 can enhance the ability of MLV-hCox2 to promote fracture repair.

A number of novel or known genes or Expressed Targeted Sequences (ESTs) has been identified in microarray studies. In vitro screening assays for bone formation, bone resorption, and angiogenesis can be used in conjunction with the direct intramedullary injection of the MLV-based vector in the rat femoral fracture model to identify that one or more of those fracture repair promoting genes that were upregulated during fracture healing.

It will be apparent that the precise details of the methods or compositions described may be varied or modified without departing from the spirit of the described invention. We claim all such modifications and variations that fall within the scope and spirit of the claims below.

1. A method for promoting repair of a bone fracture or vertebra fusion in a subject, comprising:
administering to the subject a viral vector comprising a recombinant nucleic acid encoding cyclooxygenase (Cox)-2 operably linked to a heterologous promoter;
wherein the nucleic acid encoding Cox-2 comprises a 3′ untranslated region, and wherein the 3′ untranslated region of the nucleic acid encoding Cox-2 is sufficiently truncated to stabilize an mRNA transcribed from the nucleic acid encoding Cox-2;
thereby promoting the repair of the bone fracture or the vertebra fusion in the subject.

2. The method of claim 1, wherein the viral vector is an adeno-associated viral vector.

3. The method of claim 1, wherein the vector is administered locally to the subject.

4. The method of claim 3, wherein the vector is administered to muscle interstital cells adjacent to a site of the bone fracture.

5. The method of claim 3, wherein the local administration comprises administering the vector to the bone or the vertebra to be fused in the subject.

6. The method of claim 5, wherein the local administration further comprises administering the vector to muscle interstitial cells adjacent to the bone or the vertebra to be fused in the subject.

7. The method of claim 1, wherein the vector is administered into the periosteum at a site of the bone fracture.

8. The method of claim 1, wherein the vector is administered by intramedullary injection at a site of the bone fracture.

9. The method of claim 1, wherein the vector is administered into subperiosteum at a site of the bone fracture.

10. The method of claim 1, wherein the bone fracture is repaired in the absence of extra-skeletal bone formation.

11. The method of claim 1, wherein the nucleic acid does not comprise a destabilizing element in the 3′ untranslated region.

12. The method of claim 11, wherein the destabilizing element is an adenine and uridine-rich element (ARE).

13. The method of claim 12, wherein the destabilizing element is a nucleotide comprising AUUUA.

14. The vector of claim 11, wherein the vector does not comprise the nucleotide sequence of SEQ ID NO: 17.

15. The method of claim 1, wherein the 3′ untranslated region is at most 25 nucleotides in length

16. The method of claim 15, wherein the 3′ untranslated region is at most 15 nucleotides in length.

17. The method of claim 1, wherein the vector comprises an optimized Kozak sequence operably linked to the nucleic acid encoding Cox-2.

18. The method of claim 17, wherein the optimized Kozak sequence comprises the nucleotide sequence X₁CC X₂CCA(T/U)GG (SEQ ID NO: 15), wherein X₁ and X₂ are A, T, C, or G.

19. The method of claim 1, wherein the Cox-2 is human Cox-2.

20. The method of claim 1, wherein the subject is a human.
